(19) United States
(12) Patent Application Publication
(10) Pub. No.: US 2004/0097518 A1
(43) Pub. Date: May 20, 2004

(54) Quinoline derivatives as nk-3 antagonists

(76) Inventors: Carlos Farina, Baranzate di Bollate (IT); Stefania Gagliardi, Baranzate di Bollate (IT); Giuseppe Giardina, Baranzate di Bollate (IT); Mario Grugni, Baranzate di Bollate (IT); Marisa Martinelli, Baranzate di Bollate (IT); Guy Marguerite Maria Gerard Nadler, Rennes (FR)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/432,925

(22) PCT Filed: Nov. 26, 2001
(86) PCT No.: PCT/EP01/13833

(30) Foreign Application Priority Data
Nov. 28, 2000 (GB) 0028965.5

(51) Int. Cl. A61K 31/496; C 07D 4 3/02

(52) U.S. Cl. 514/253.06; 544/363

(57) ABSTRACT

Certain compounds of formula (I) below or a pharmaceutically acceptable salt or hydrate thereof:

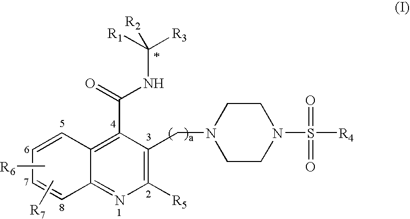

a process for preparing such compounds, a pharmaceutical composition comprising such compounds and the use of such compounds and composition in medicine.

The present invention relates to novel compounds, in particular to novel quinoline derivatives, to processes for the preparation of such compounds, to pharmaceutical compositions containing such compounds and to the use of such compounds in medicine.

The mammalian peptide Neurokinin B (NKB) belongs to the Tachykinin (TK) peptide family which also include Substance P (SP) and Neurokinin A (NKA). Pharmacological and molecular biological evidence has shown the existence of three subtypes of TK receptor (NK₁, NK₂ and NK₃) and NKB binds preferentially to the NK₃ receptor although it also recognises the other two receptors with lower affinity (Maggi et al, 1993, J. Auton. Pharmacol, 13, 23-93).

Selective peptidic NK₃ receptor antagonists are known (Drapeau, 1990 Regul. Pept., 31, 125-135), and findings with peptidic NK₃ receptor agonists suggest that NKB, by activating the NK₃ receptor, has a key role in the modulation of neural input in airways, skin, spinal cord and nigro-striatal pathways (Myers and Undem, 1993, J. Physiol., 470, 665-679; Counture et al., 1993, Regul. Peptides, 46,426-429; Mccarson and Krause, 1994, J Neurosci., 14 (2), 712-720; Arenas et al. 1991, J. Neurosci., 11, 2332-8). However, the peptide-like nature of the known antagonists makes them likely to be too labile from a metabolic point of view to serve as practical therapeutic agents.

International Patent Application, Publication number WO 00/31037 discloses certain compounds stated to be non-peptide NK-3 antagonists and also to have NK-2 antagonist activity. These compounds are disclosed to be of potential use in the prevention and treatment of a wide variety of clinical conditions, which are characterised by overstimulation of the Tachykinin receptors, in particular NK-3 and NK-2.

We have now discovered a further novel class of potent non-peptide NK-3 antagonists some of which fall within the generic scope of WO 00/31037. The new compounds are also far more stable from a metabolic point of view than the known peptidic NK-3 receptor antagonists and are of potential therapeutic utility. The new compounds also have good NK-2 antagonist activity and are therefore considered to be of potential use in the prevention and treatment of a wide variety of clinical conditions which are characterised by overstimulation of the Tachykinin receptors, in particular NK-3 and NK-2.

These conditions include respiratory diseases, such as chronic obstructive pulmonary disease (COPD), asthma, airway hyper-reactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease, irritable bowel syndrome (IBS), gastro-exophageous reflex disease (GERD); urinary incontinence and disorders of the bladder function; renal disorders; increased blood pressure, proteinuria, coagulopathy and peripheral and cerebral oedema following pre-eclampsia in pregnancies (hereinafter referred to as the ‘Primary Conditions’).

Certain of these compounds also show CNS activity and hence are considered to be of particular use in the treatment of disorders of the central nervous system such as anxiety, depression, psychosis and schizophrenia; neurodegenerative disorders such as AIDS related dementia, senile dementia of the Alzheimer type, Alzheimer's disease, Down's syndrome, Huntingdon's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilatation and vasospastic diseases such as angina, migraine and Reynaud's disease and pain or nociception, for example, that is attributable to or associated with any of the foregoing conditions especially the transmission of pain in migraine, (hereinafter referred to as the ‘secondary Conditions’).

The new compounds also show improved oral bioavailability.

The compounds of formula (I) are also considered to be useful as diagnostic tools for assessing the degree to which neurokinin-3 and neurokinin-2 receptor activity (normal, overactivity or underactivity) is implicated in a patient's symptoms.

According to the present invention, there is provided a compound of formula (I) below or a pharmaceutically acceptable salt or hydrate thereof:

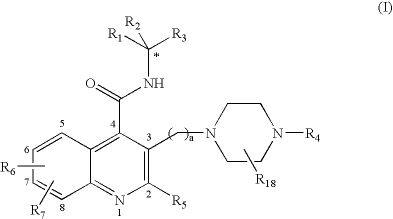

wherein:

R₁ is H or alkyl;

R₂ is aryl or cycloalkyl or heteroaryl, optionally substituted one or more times by alkyl, OH or alkoxy;

R₃ is H or alkyl or cycloalkyl or cycloalkylalkyl, optionally substituted one or more times by hydroxy or by one or more fluorines;

R₄ is H, or —R₈R₉ where R₈ is optionally substituted one or more times by R₁₃, or R₁₉;

R₈ is alkyl or alkenyl;

R₉ is S(O₂)R₁₀, S(O₂)OR₁₀, ONO, C(O)OR₁₀, C(O)NR₁₁R₁₂, or CN;

R₁₀ is H, alkyl, aryl or cycloalkyl;

R₁₁ and R₁₂ are independently selected from H and alkyl;

R₁₃ is R₁₄ or —R₁₄R₁₅;

R₁₄ is alkyl, aryl, cycloalkyl, arylalkyl, or a five-, six-, seven- or eight-membered heterocyclic ring comprising one or more heteroatoms selected from N, O and S;

R₁₅ is alkyl or —R₁₆COOR₁₇;

R₁₆ is a single bond or alkyl;

R₁₇ is H or alkyl;

R₁₈ is H or up to three oxo substituents;

R₁₉ is R₂₀ or —R₂₀R₂₁;

R₂₀ is alkyl, alkenyl or a single bond;

R₂₁ is OH, aryl, cycloalkyl or a saturated heterocyclic ring comprising one or more heteroatoms selected from N, O and S;

R₅ is a alkyl, cycloalkyl, cycloalkylalkyl, aryl, or single or fused ring aromatic heterocyclic group, which group may be substituted one or more times by halo, hydroxy, alkyl or alkyl substituted one or more times by halo or hydroxy;

R₆ represents H or up to three substituents independently selected from the list consisting of: alkyl, alkenyl, aryl, alkoxy or a hydroxylated derivative thereof, hydroxy, halogen, nitro, cyano, carboxy, alkylcarboxy, alkylcarboxyalkyl, haloalkyl such as trifluoromethyl, amino or mono- or di- alkylamino; or R₆ represents a bridging moiety which is arranged to bridge two adjacent ring atoms, which bridging moiety comprises alkyl or dioxyalkylene;

R₇ is H or halo;

a is 1-6; and

any of R₂, R₅, R₈, R₁₀, R₁₁, R₁₂, R₁₄, R₁₆, R₁₇ and R₂₁ may optionally be substituted one or more times by halo, hydroxy, amino, cyano, nitro, carboxy or oxo;

subject to the proviso that said compound is not a compound wherein R₇ represents H, R₅ represents unsubstituted phenyl, R₁₈ is H, and R₁, R₂, R₃ and R₄ are one of the following combinations:

| 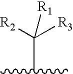 | a | R₄ | R₆ |
|---|---|---|---|
| 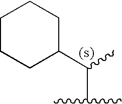 | 1 | 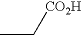 | H |
| 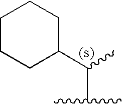 | 1 | H | H |
| 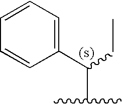 | 1 | H | H |
| 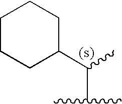 | 1 | 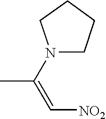 | H |
| 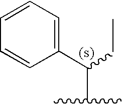 | 2 | 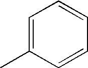 | H |
| 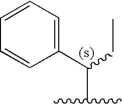 | 3 | 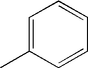 | H |
| 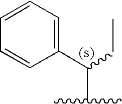 | 4 | 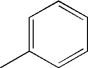 | H |
| 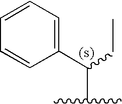 | 3 | 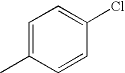 | H |
| 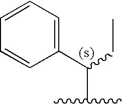 | 2 | 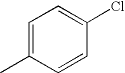 | H |
| 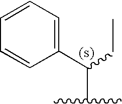 | 2 | 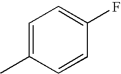 | H |
| 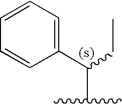 | 3 | 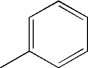 | OMe |
| 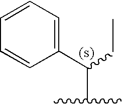 | 1 | 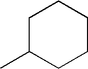 | H |
| 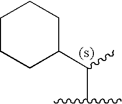 | 1 | 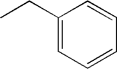 | H |
| 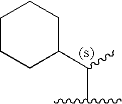 | 1 | 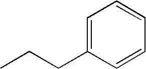 | H |
| 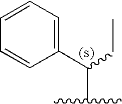 | 1 | 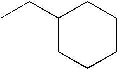 | H |
| 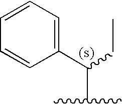 | 1 | 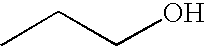 | H |
| 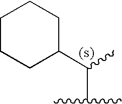 | 1 | 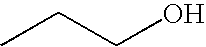 | H |
| 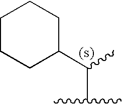 | 1 | Et | H |
| 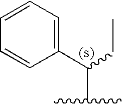 | 1 | Me | H |
| 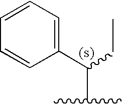 | 1 | 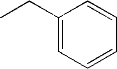 | H |
| 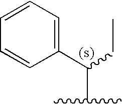 | 2 | Me | H |
| 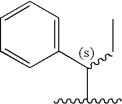 | 1 | Et | H |
| 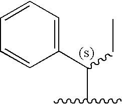 | 1 | 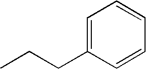 | H |
| 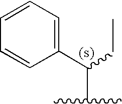 | 3 | 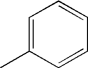 | OH |

Suitably, R₃ is C₁-₆ alkyl, such a methyl, ethyl, iso-propyl, cyclopropyl, hydroxymethyl or hydroxyethyl.

Advantageously, R₂ may represent phenyl or cyclohexyl. In some preferred embodiments, R₂ represents phenyl which is substituted, suitably meta- or para-substituted, once by —OMe or —OH.

Preferably, R₁ may be hydrogen. Alternatively, R₁ may be methyl.

Suitably, R₅ may be unsubstituted phenyl. Alternatively, R₅ may be phenyl which is substituted one or more times by halo such as fluoro, or by an alkyl group which may be substituted one or more times by halo such as fluoro. Said R₅ may be phenyl which is substituted once by trihalomethyl, such as trifluoromethyl. As yet a further alternative, R₅ may be a heterocyclic ring, such as an unsaturated heterocyclic ring such as a five-membered unsaturated heterocyclic ring which comprises at least one S or N heteroatom. Said R₅ may for example be

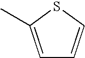

Advantageously, R₇ may represent hydrogen.

Optionally, R₆ may represent hydrogen. Alternatively, R₆ may represent or one or more substituents selected from fluoro, chloro, bromo or trifluoromethyl. Preferably, each of said one or more substituents may be respectively positioned at the 5′, 6′, 7′ or 8′ position around the quinoline ring of said compound. As yet a further alternative, R₆ may represent one or more substituents selected from hydroxy, alkoxy such as methoxy or ethoxy or a hydroxylated derivative thereof, alkoxycarboxylate such as methoxycarboxylate or ethoxycarboxylate or an esterified derivative thereof such as methoxyethanoate ethoxyethanoate, or alkoxyamido such as methoxyamido or ethoxyamido. In particular, R₆ may represent one or more substituents selected from methyl, methoxy, ethyl, and ethoxy; preferably methoxy. Suitably, said one or more substituents may be located at the 6 and/or the 7 position around the quinoline ring. As yet a further alternative, said R₆ may represent a bridging substituent which is dioxyethylene, which bridging substituent is arranged to bridge the 6 and 7 positions around said quinoline ring.

Advantageously, a is 1, 2 or 3. Most preferably, a is 1.

In one preferred aspect of the invention, R₄ represents hydrogen.

In another preferred aspect of the invention, R₄ represents —R₈R_{9.}

Suitably, R₈ may be methyl or ethyl. Alternatively, R₈ may be ethenyl or propenyl.

In some favourable embodiments, R₉ may be C(O)OH or C(O)NH₂. Alternatively, R₉ may be S(O₂)R₁₀, S(O₂)OR₁₀, or C(O)OR₁₀, and R₁₀ may be phenyl, methyl or ethyl. As yet a further alternative, R₉ may be C(O)NR₁₁R₁₂ and R₁₀ and R₁₁ may each be the same one of methyl or ethyl.

Advantageously, R₄ is branched or linear R₈(R₁₃)R₉, where R₁₃ is R₁₄ or —R₁₄R₁₅. Suitably R₁₄ is a five- or six-membered saturated heterocyclic ring. Said heterocyclic ring may comprise one or more N atoms. Optionally, said heterocyclic ring may be N-linked to said R₈. Alternatively, R₁₄ may be C₁-₆ alkyl, or phenyl, or phenylmethyl, or phenylethyl. Said R₁₅ may be methylethanoate, ethylethanoate, propylethanoate or butylethanoate.

In yet another aspect of the invention, R₄ is R₁₉ which is R₂₀ or —R₂₀R₂₁ . In some embodiments, R₂₀ is a single bond and R₂₁ is aryl such as phenyl. In other embodiments R₂₀ is straight chain alkyl such as methyl, ethyl or propyl and R₂₁ is OH, aryl, or a saturated heterocyclic ring comprising one or more N heteroatoms. In yet further embodiments, R₂₀ is straight chain alkyl such as methyl, ethyl or propyl.

Suitably, R₁₈ may be H. Alternatively, R₁₈ may represent one or more oxo substituents. In many preferred embodiments, R₁₈ represents one oxo substituent which is positioned at the 3′, 5′ or 6′ position around the piperazine ring of the compound of formula (I). In other preferred embodiments, R₁₈ represents two oxo substituents which are respectively positioned at the 3′ and 5′ or at the 3′ and 6′ positions around the piperazine ring of the compound of formula (I).

In especially preferred embodiments, the compound of the present invention is selected from the following:

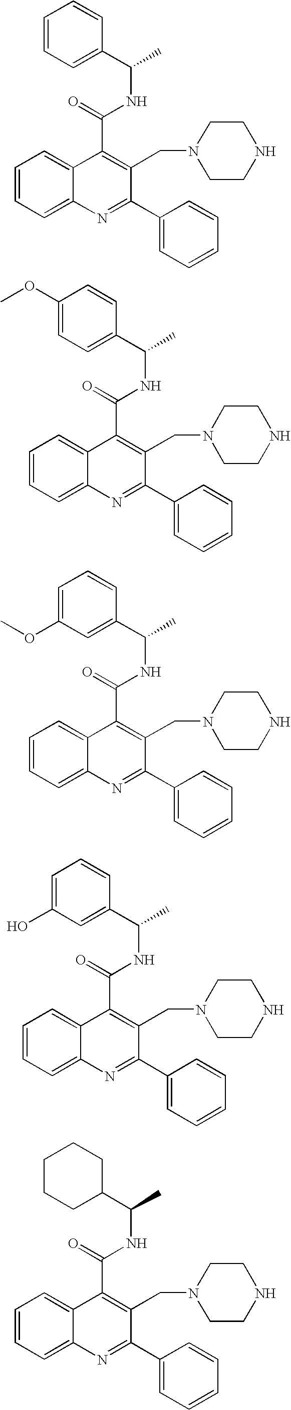

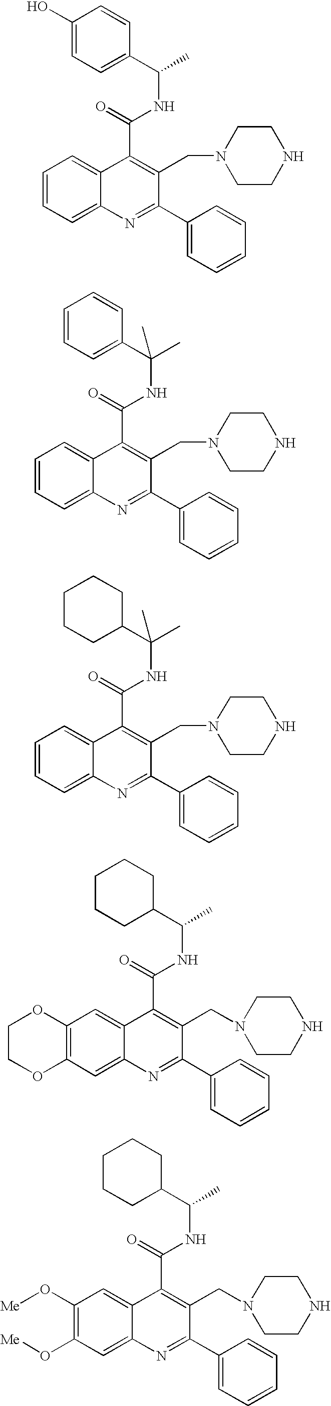

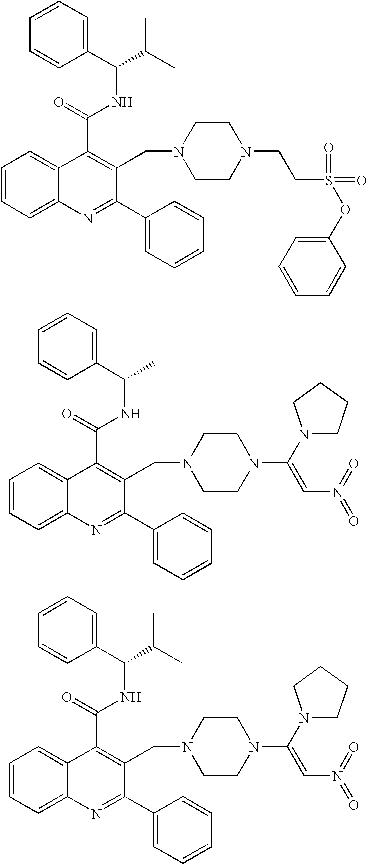

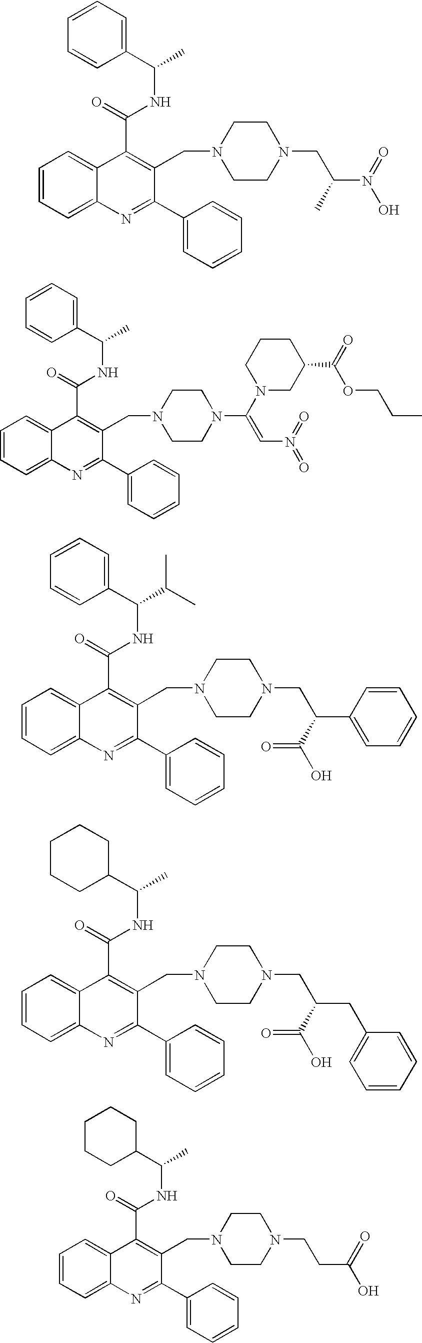

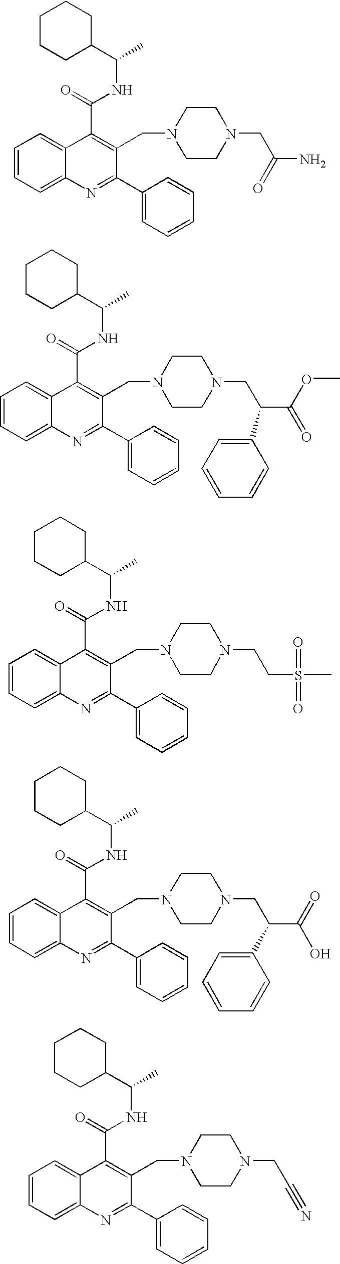

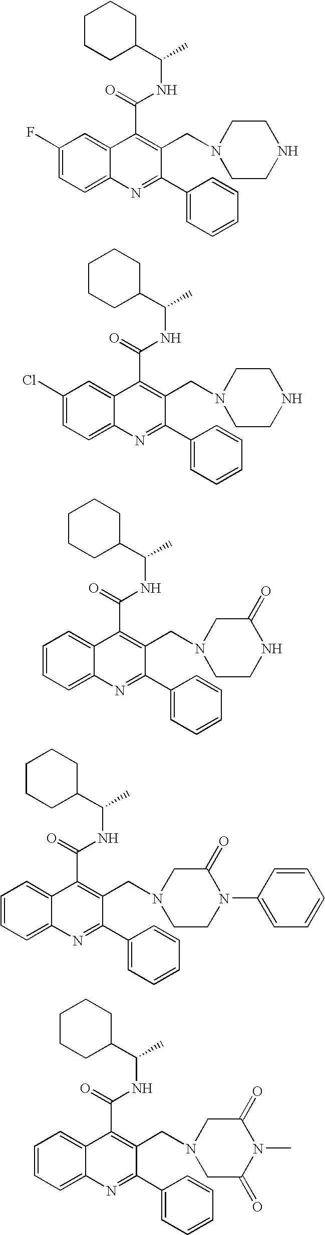

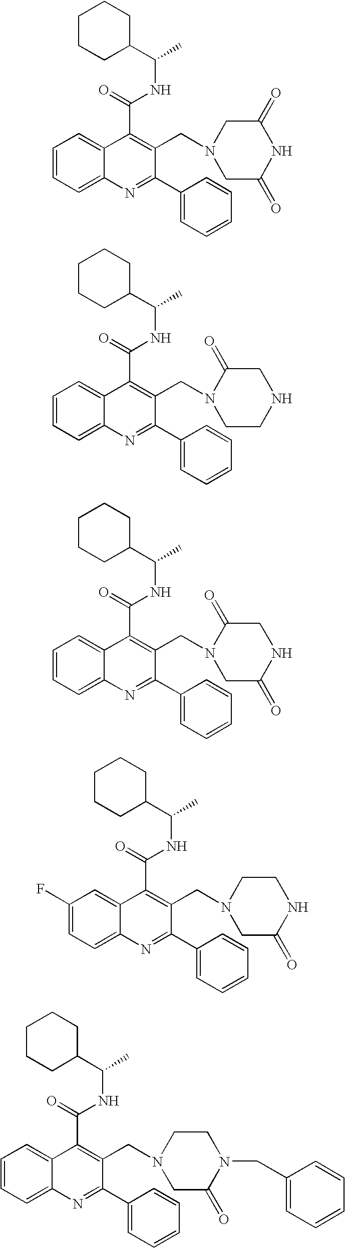

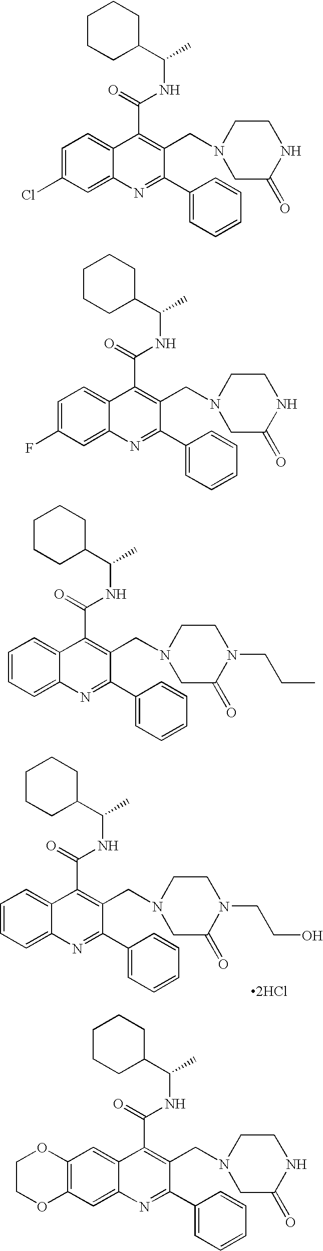

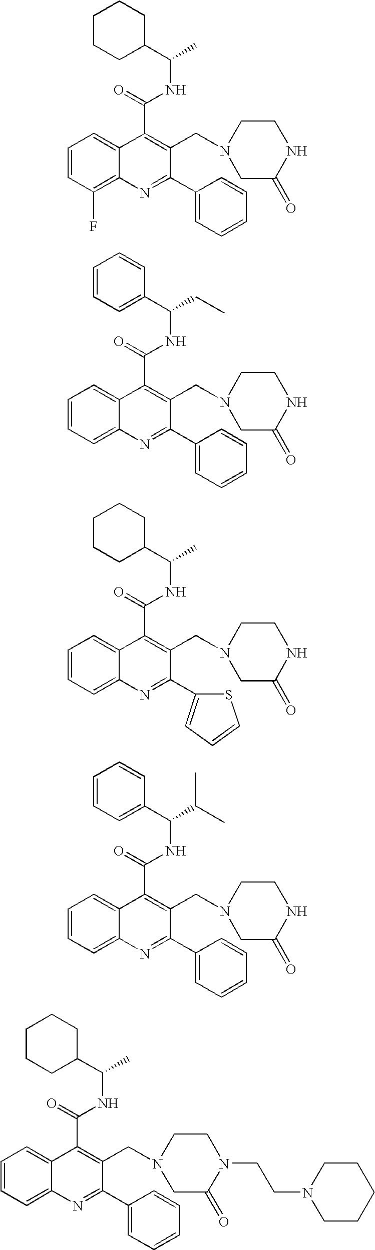

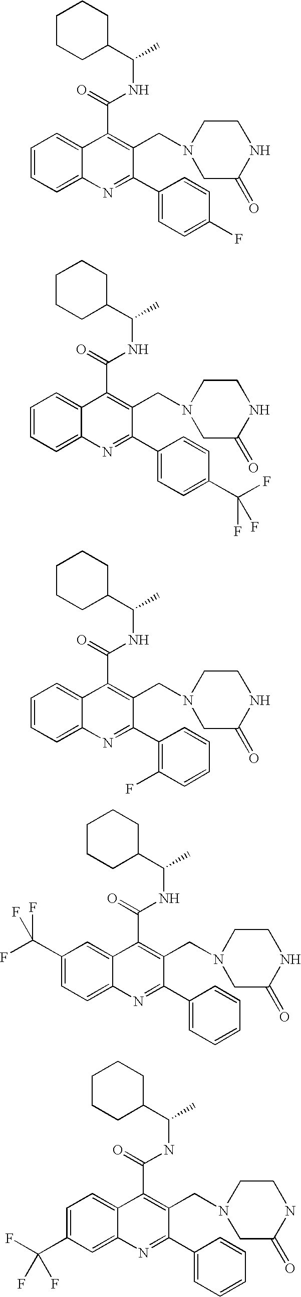

In particularly preferred embodiments, the compound of the present invention is selected from the following:

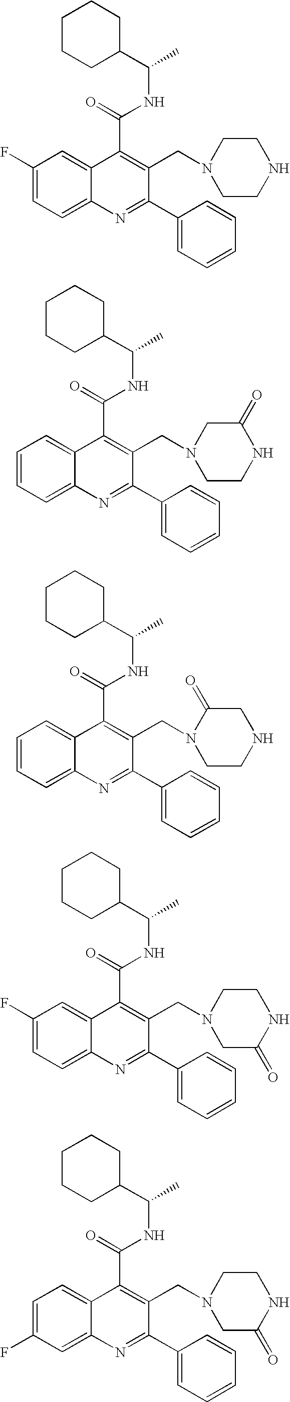

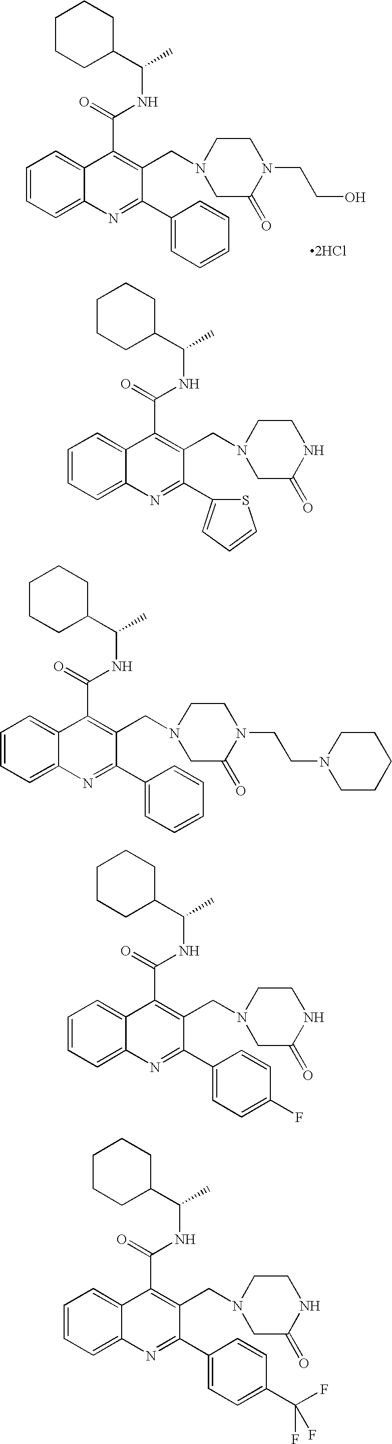

The compounds of formula (I) may have at least one asymmetric centre—for example the carbon atom labelled with an asterisk (*) in the compound of formula (I)—and therefore may exist in more than one stereoisomeric form. The invention extends to all such stereoisomeric forms and to mixtures thereof, including racemates. In particular, the invention includes compounds wherein the asterisked carbon atom in formula (I) has the stereochemistry shown in formula (Ia):

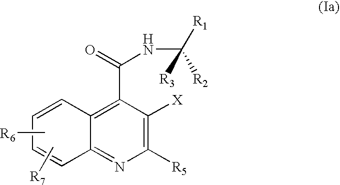

wherein R₁, R₂, R₃, R₅, R₆, and R₇ are as defined in relation to formula (I), and X represents the moiety

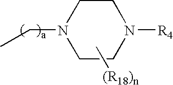

The compounds of formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels.

A substantially pure form will generally contain at least 50% (excluding normal pharmaceutical additives), preferably 75%, more preferably 90% and still more preferably 95% of the compound of formula (I) or its salt or solvate.

One preferred pharmaceutically acceptable form is the crystalline form, including such form in pharmaceutical composition. In the case of salts and solvates the additional ionic and solvent moieties must also be non-toxic.

Suitable salts are pharmaceutically acceptable salts.

Suitable pharmaceutically acceptable salts include the acid addition salts with the conventional pharmaceutical acids, for example maleic, hydrochloric, hydrobromic, phosphoric, acetic, fumaric, salicylic, citric, lactic, mandelic, tartaric, succinic, benzoic, ascorbic and methanesulphonic.

Suitable pharmaceutically acceptable salts include salts of acidic moieties of the compounds of formula (I) when they are present, for example salts of carboxy groups or phenolic hydroxy groups.

Suitable salts of acidic moieties include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-β-phenethylamine, dehydroabietylamine, N,N′-bisdehydroabietylamine, glucamine, N-methylglucamine or bases of the pyridine type such as pyridine, collidine, quinine or quinoline.

Suitable solvates are pharmaceutically acceptable solvates.

Suitable pharmaceutically acceptable solvates include hydrates.

The term ‘alkyl’ (unless specified to the contrary) when used alone or when forming part of other groups (such as the ‘alkoxy’ group) denotes straight- or branched-chain alkyl groups containing 1 to 12 carbon atoms, suitably 1 to 6 carbon atoms, examples include methyl, ethyl, n-propyl, isopropyl, n-butyl, isobutyl or tert-butyl group.

The term ‘carbocylic’ denotes cycloalkyl and aryl rings.

The term ‘cycloalkyl’ denotes groups having 3 to 12, suitably 4 to 6 ring carbon atoms.

The term ‘aryl’ denotes aromatic groups including phenyl and naphthyl, preferably phenyl which unless specified to the contrary optionally comprise up to five, preferably up to three substituents selected from halogen, alkyl, phenyl, alkoxy, haloalkyl, hydroxyalkyl, hydroxy, amino, nitro, cyano, carboxy, alkoxycarbonyl, alkoxycarbonylalkyl, alkylcarbonyloxy, or alkylcarbonyl groups.

The term ‘aromatic heterocyclic group’ denotes groups comprising aromatic heterocyclic rings containing from 5 to 12 ring atoms, suitably 5 or 6, and comprising up to four hetero-atoms in the or each ring selected from S, O or N.

Unless specified to the contrary, suitable substituents for any heterocyclic group includes up to 4 substituents selected from the group consisting of: alkyl, alkoxy, aryl and halogen or any two substituents on adjacent carbon atoms, together with the carbon atoms to which they are attached, may form an aryl group, preferably a benzene ring, and wherein the carbon atoms of the aryl group represented by the said two substituents may themselves be substituted or unsubstituted.

It will be understood that, unless otherwise specified, groups and substituents forming part of a compound in accordance with the invention are unsubstituted.

When used herein the term “halogen” or “halo” refers to fluorine, chlorine, bromine and iodine, preferably fluorine, chlorine or bromine.

When used herein the term “acyl” includes residues of acids, in particular a residue of a carboxylic acid such as an alkyl- or aryl- carbonyl group.

The invention also provides in one aspect a process for the preparation of a compound of formula (I), or a salt thereof and/or a solvate thereof, which process comprises reacting a compound of formula (II) or an active derivative thereof:

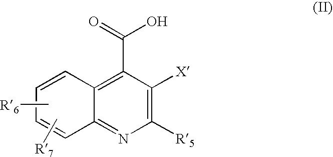

wherein R′₆, R′₇, R′₅ and X′ are R₆, R₇, R₅ and X respectively as hereinbefore defined in relation to formula (I) or (Ia), or a group convertible to R₆, R₇, R₅ and X respectively; with a compound of formula (III):

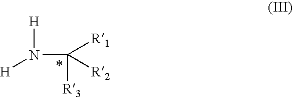

wherein R′₁, R′₂, and R′₃ are R₁, R₂, and R₃ as defined for formula (I) or a group or atom convertible to R₁, R₂, and R₃ respectively; to form a compound of formula (Ib):

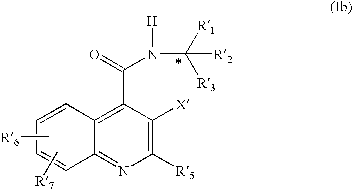

wherein R′₁, R′₂, R′₃, X′, R′₅, R′₆ and R′₇ are as defined above, and thereafter carrying out one or more of the following optional steps:

(i) converting any one of R′₁, R′₂, R′₃, X′, R′₅, R′₆ and R′₇ to R₁, R₂, R₃, X, R₅, R₆ and R₇ respectively as required, to obtain a compound of formula (I);

(ii) converting a compound of formula (I) into another compound of formula (I); and

(iii) preparing a salt of the compound of formula (I) and/or a solvate thereof.

Suitable groups convertible into other groups include protected forms of said groups.

Suitably R′₁, R′₂, R′₃, X′, R′₅, R′₆ and R′₇ each represents R₁, R₂, R₃, X, R₅, R₆ and R₇ respectively or a protected form thereof.

It is favoured if the compound of formula (II) is present as an active derivative.

A suitable active derivative of a compound of formula (II) is a transient activated form of the compound of formula (II) or a derivative wherein the carboxy group of the compound of formula (II) has been replaced by a different group or atom, for example by an acyl halide, preferably a chloride, or an acylazide or a carboxylic acid anhydride.

Other suitable active derivatives include: a mixed anhydride formed between the carboxyl moiety of the compound of formula (II) and an alkyl chloroformate; an activated ester, such as a cyanomethyl ester, thiophenyl ester, p-nitrophenyl ester, p-nitrothiophenyl ester, 2,4,6-trichlorophenyl ester, pentachlorophenyl ester, pentafluorophenyl ester, N-hydroxy-phtalimido ester, N-hydroxypiperidine ester, N-hydroxysuccinimide ester, N-hydroxy benzotriazole ester; alternatively, the carboxy group of the compound of formula (II) may be activated using a carbodiimide or N,N′-carbonyldiimidazole.

The reaction between the compound of formula (II) or the active derivative thereof and the compound of formula (III) is carried out under the appropriate conventional conditions for the particular compounds chosen. Generally, when the compound of formula (II) is present as an active derivative the reaction is carried out using the same solvent and conditions as used to prepare the active derivative, preferably the active derivative is prepared in situ prior to forming the compound of formula (Ib) and thereafter the compound of formula (I) or a salt thereof and/or a solvate thereof is prepared.

For example, the reaction between an active derivative of the compound of formula (II) and the compound of formula (III) may be carried out:

(a) by first preparing an acid chloride and then coupling said chloride with the compound of formula (III) in the presence of an inorganic or organic base in a suitable aprotic solvent such as dimethylformamide (DMF) at a temperature in a range from −70 to 50° C. (preferably in a range from −10 to 20° C.); or

(b) by treating the compound of formula (II) with a compound of formula (III) in the presence of a suitable condensing agent, such as for example N,N′-carbonyl diimidazole (CDI) or a carbodiimide such as dicyclohexylcarbodiimide (DCC) or N-dimethylaminopropyl-N′-ethylcarbodiimide, preferably in the presence of N-hydroxybenzotriazole (HOBT) to maximise yields and avoid racemization processes (see Synthesis, 453, 1972), or O-benzotriazol-1-yl-N,N,N′,N′-tetramethyluroniumhexafluorophosphate (HBTU), in an aprotic solvent, such as a mixture of acetonitrile (MeCN) and tetrahydrofuran (THF), for example a mixture in a volume ratio of from 1:9 to 7:3 (MeCN:THF), at any temperature providing a suitable rate of formation of the required product, such as a temperature in the range of from −70 to 50° C., preferably in a range of from −10 to 25° C., for example at 0° C.

A preferred reaction is set out in Scheme 1 shown below:

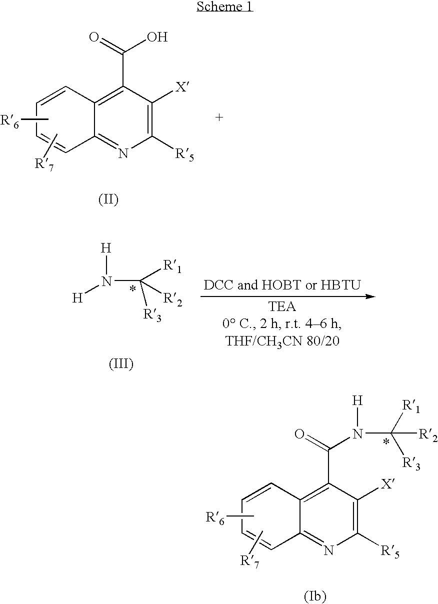

wherein R′₁, R′₂, R′₃, X′, R′₅, R′₆ and R′₇ are as defined above.

In the case in which the corresponding alkyl (such as methyl or ethyl) ester of compound (II) is utilised, an hydrolysis to compound (II) is required before conversion to compound (Ib) in Scheme 1. Such hydrolysis can be carried out under acidic conditions, such 10-36% hydrochloric acid at a temperature in the range between 30 and 100° C.

It will be appreciated that a compound of formula (Ib) may be converted to a compound of formula (I), or one compound of formula (I) may be converted to another compound of formula (I) by interconversion of suitable substituents. Thus, certain compounds of formula (I) and (Ib) are useful intermediates in forming other compounds of the present invention.

Accordingly, in a further aspect the invention provides a process for preparing a compound of formula (I), or a salt thereof and/or a solvate thereof, which process comprises converting a compound of the above defined formula (Ib) wherein at least one of R′₁, R′₂, R′₃, X′, R′₅, R′₆ and R′₇ is not R₁, R₂, R₃, X, R₅, R₆ or R₇ respectively, thereby to provide a compound of formula (I); and thereafter, as required, carrying out one or more of the following optional steps:

(i) converting a compound of formula (I) into another compound of formula (I); and

(ii) preparing a salt of the compound of formula (I) and/or a solvate thereof.

Suitably, in the compound of formula (Ib) the variables R′₁, R′₂, R′₃, X′, R′₅, R′₆ and R′₇ are R₁, R₂, R₃, X, R₅, R₆ and R₇ respectively or they are protected forms thereof.

The above mentioned conversions, protections and deprotections are carried out using the appropriate conventional reagents and conditions and are further discussed below.

A chiral compound of formula (III) wherein R₂ is a C₅ or C₇ cycloalkyl group, R₃ is methyl and R₁ is H are described in J. Org. Chem. (1996), 61 (12), 4130-4135. A chiral compound of formula (III) wherein R₂ is phenyl, R₃ is isopropyl and R₁ is H is a known compound described in for example Tetrahedron Lett. (1994), 35(22), 3745-6.

The compounds of formula (III) are known commercially available compounds or they can be prepared from known compounds by known methods, or methods analogous to those used to prepare known compounds, for example the methods described in Liebigs Ann. der Chemie, (1936), 523, 199.

In some embodiments of the invention, a compound of formula (II) or the corresponding alkyl (such as methyl or ethyl) ester is prepared by reacting a compound of formula (IV) or the corresponding alkyl (such as methyl or ethyl) ester:

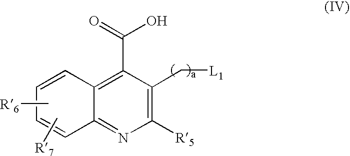

wherein R′₆, R′₇, R′₅ and a are as defined above and L₁ represents a halogen atom such as a bromine atom, with a compound of formula (V):

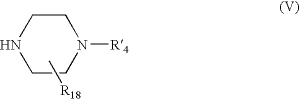

wherein R′₄ is R₄ as defined in relation to formula (I) or a protected form thereof.

Suitably, R′₄ is R_{4.}

Suitably, reaction between the compounds of formulae (IV) or the corresponding alkyl (such as methyl or ethyl) ester and (V) is carried out under conventional amination conditions, for example when L₁ is a bromine atom then the reaction is conveniently carried out in an aprotic solvent, such as tetrahydrofuran or dimethylformamide at any temperature providing a suitable rate of formation of the required product, usually at ambient temperature; preferably the reaction is carried out in the presence of triethylamine (TEA) or K₂CO_{3.}

The compounds of formula (V) are known, commercially available compounds or they can be prepared using methods analogous to those used to prepare known compounds; for example the methods described in the Chemistry of the Amino Group, Patais (Ed.), Interscience, New York 1968; Advanced Organic Chemistry, March J, John Wiley & Sons, New York, 1992 ; J. Heterocyclic Chem. (1990), 27, 1559; Synthesis (1975), 135, Bioorg. Med. Chem. Lett. (1997), 7, 555, or Protective Groups in Organic Synthesis (second edition), Wiley Interscience, (1991) or other methods mentioned herein.

In cases where a is 1, a compound of formula (IV) or the corresponding alkyl (such as methyl or ethyl) ester may be prepared by appropriate halogenation of a compound of formula (VI) or the corresponding alkyl (such as methyl or ethyl) ester:

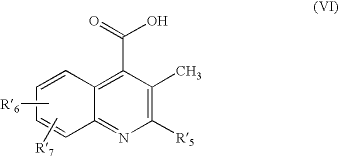

wherein R′₆, R′₇ and R′₅ are as defined above in relation to formula (II).

Suitable halogenation reagents are conventional reagents depending upon the nature of the halogen atom required, for example when L₁ is bromine a preferred halogenation reagent is N-bromosuccinimide (NBS).

The halogenation of the compound of formula (VI) or the corresponding alkyl (such as methyl or ethyl) ester is suitably carried out under conventional conditions, for example bromination is carried out by treatment with NBS in an inert solvent, such as carbon tetrachloride CCl₄, or 1,2-dichloroethane or CH₃CN, at any temperature providing a suitable rate of formation of the required product, suitably at an elevated temperature such as a temperature in the range of 60° C. to 100° C., for example 80° C.; preferably the reaction is carried out in the presence of a catalytic amount of benzoyl peroxide.

A compound of formula (VI) is conveniently prepared by reacting a compound of formula (VII):

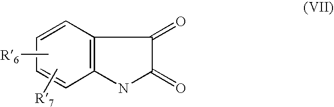

wherein R′₆ and R′₇ are as defined in relation to formula (II), with a compound of formula (XIII):

R₅′—CO—CH₂—Me (XIII)

wherein R′₅ is as defined in relation to formula (II).

The reaction between the compounds of formula (VII) and (XIII) is conveniently carried out using Pfitzinger reaction conditions (see for example J. Prakt. Chem. 33, 100 (1886), J. Prakt. Chem. 38, 582 (1888), J. Chem. Soc. 106 (1948) and Chem. Rev. 35, 152 (1944)), for example in an alkanolic solvent such as ethanol, at any temperature providing a suitable rate of formation of the required product, but generally at an elevated temperature, such as the reflux temperature of the solvent, and preferably in the presence of a base such as potassium hydroxide or potassium tert-butoxide.

The compounds of formula (VII) are known compounds or they are prepared according to methods used to prepare known compounds for example those disclosed in J. Org. Chem. 21, 171 (1955); J. Org. Chem. 21, 169 (1955).

Alternatively a compound of formula (VI) may be conveniently prepared by reacting a compound of formula (XIV)

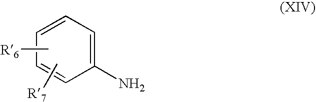

wherein R′₆ and R′₇ are as defined in relation to formula (II), with a compound of formula (XV):

R₅′—CHO (XV)

wherein R′₅ is as defined in relation to formula (II) in presence of oxobutyric acid.

The reaction between the compounds of formula (XIV) and (XV) is conveniently carried out using Doebner reaction conditions (see for example Chem. Ber. 29, 352 (1894); Chem. Revs. 35, 153, (1944); J. Chem. Soc. B, 1969, 805), for example in an alcoholic solvent such as ethanol, at any temperature providing a suitable rate of formation of the required product, but generally at an elevated temperature, such as the reflux temperature of the solvent.

The compounds of formula (XIV) and (XV) are known compounds or they are prepared according to methods used to prepare known compounds for example as described in Vogel's Textbook of Practical Organic Chemistry.

In some alternative embodiments of the invention, a compound of formula (II) wherein X′ represents

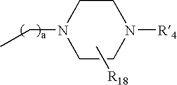

is prepared by reacting a compound of formula (VII) as defined above with a compound of formula (VIII):

R₅′—CO—CH₂—(CH₂)a-T₅ (VIII)

wherein R′₅ is as defined in relation to formula (II), and T₅ is a group

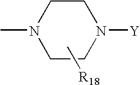

where Y is a protecting group such as a benzyl group, particularly a protecting group which is stable in basic conditions such as a terbutoxycarbonyl group, or a group R₄ as defined in relation to formula (I) or a protected form thereof or a group convertible thereto, and a is as defined in relation to formula (II); and thereafter as required removing any protecting group, for example by dehydrogenation, and/or converting any group T₅ to

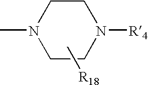

The reaction between the compounds of formula (VII) and (VIII) is conveniently carried out using Pfitzinger reaction conditions (see for example J. Prakt. Chem. 33, 100 (1886), J. Prakt. Chem. 38, 582 (1888), J. Chem. Soc. 106 (1948) and Chem. Rev. 35, 152 (1944)), for example in an alkanolic solvent such as ethanol, at any temperature providing a suitable rate of formation of the required product, but generally at an elevated temperature, such as the reflux temperature of the solvent, and preferably in the presence of a base such as potassium hydroxide or potassium tert-butoxide.

Protected forms of

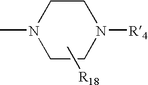

will vary according to the. particular nature of the group being protected but will be chosen in accordance with normal chemical practice.

Groups convertible to

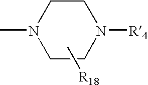

include groups dictated by conventional chemical practice to be required and to be appropriate, depending upon the specific nature of the

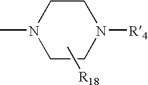

under consideration.

Suitable deprotection methods for deprotecting protected forms of

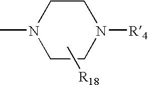

and conversion methods for converting T₅ to

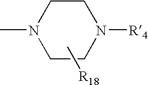

will be those used conventionally in the art depending upon the particular groups under consideration with reference to standard texts such as Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994 and Chemistry of the Amino Group, Patais (Ed.), Interscience, New York 1968; or Advanced Organic Chemistry, March J, John Wiley & Sons, New York, 1992.

A compound of formula (VIII) is prepared from a compound of formula (IX):

R₅′—CO—CH₂—(CH₂)ₐ—OH (IX)

wherein R′₅ is as defined in relation to formula (II) and a is as defined in relation to formula (VIII), by first halogenating, preferably brominating, or mesylating the compound of formula (IX) and thereafter reacting the halogenation or mesylation product so formed with a compound capable of forming a group T₅ so as to provide the required compound of formula (VII).

When T₅ is a group

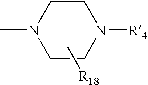

a compound capable of forming a group T₅ is a compound of the above defined formula (V).

The halogenation of the compound of formula (IX) is suitably carried out using a conventional halogenation reagent. Mesylation is conveniently carried out using mesyl chloride in an inert solvent such as methylene dichloride, at a temperature below room temperature, such as 0° C., preferably in the presence of triethylamine.

The reaction conditions between the compound of formula (IX) and the compound capable of forming a group T₅ will be those conventional conditions dictated by the specific nature of the reactants, for example when the T₅ required is a group

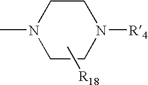

and the required compound capable of forming a group T₅ is a compound of the above defined formula (V), then the reaction between the halogenation or mesylation product of the compound of formula (IX) and the compound of formula (V) is carried out under analogous conditions to those described for the reaction between the compounds of formulae (IV) and (V).

Other compounds capable of forming a group T₅ will depend upon the particular nature of T₅, but will be those appropriate compounds dictated by conventional chemical practice with reference to standard texts such as Chemistry of the Amino Group, Patais (Ed.), Interscience, New York 1968; and Advanced Organic Chemistry, March J, John Wiley & Sons, New York, 1992.

A compound of formula (IX) may be prepared by reacting a compound of formula (X):

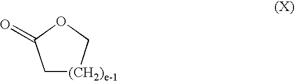

wherein a is as defined in relation to formula (VIII), with a lithium salt of formula (XI):

R′₅Li (XI)

wherein R′₅ is as defined in relation to formula (II).

The reaction between the compounds of formulae (X) and (XI) can be carried out in an aprotic solvent, such as diethyl-ether at any temperature providing a suitable rate of formation of the required product, usually at a low temperature such as in the range of −10° C. to −30° C., for example −20° C.

The compounds of formula (VII) are known compounds or they are prepared according to methods used to prepare known compounds for example those disclosed in J. Org. Chem. 21, 171(1955); J. Org. Chem. 21, 169 (1955).

The compounds of formula (X) and (XI) are known compounds or they are prepared according to methods used to prepare known compounds for example those disclosed by Krow G. R. in Organic Reactions, Vol 43, page 251, John Wiley & Sons Inc. 1994 (for the compounds of formula (X)) and Organometallics in Synthesis, Schlosser M.(Ed), John Wiley & Sons Inc. 1994 (for the compounds of formula (XI)).

In another aspect, the present invention provides a process for the preparation of a compound of formula (I), or a salt thereof and/or a solvate thereof, wherein a is 1, which process comprises reacting a compound of formula (XVI):

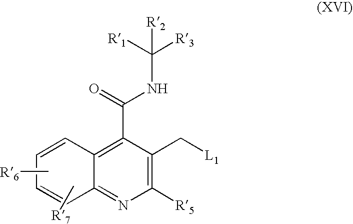

wherein each of R′₁, R′₂, R′₃, R′₅, R′₆, and R′₇ is respectively R₁, R₂, R₃, R₅, R₆, or R₇ as defined above or a group convertible to R₁, R₂, R₃, R₅, R₆, or R₇ respectively as defined above providing R′₂ is not aromatic in character, and L₁ represents a halogen atom such as a bromine atom, with a compound of formula (XVII):

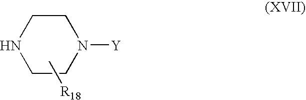

wherein Y is a protecting group such as a benzyl group, particularly a protecting group which is stable in basic conditions such as a terbutoxycarbonyl group, or a group R′₄, where R′₄ is R₄ as defined in relation to formula (I) or a protected form thereof or a group convertible thereto; and thereafter as required removing any protecting group Y, for example by dehydrogenation, and replacing the protective group Y with a group R′₄; and thereafter carrying out one or more of the following optional steps:

(i) converting any one of R′₁, R′₂, R′₃, R′₄, R′₅, R′₆ and R′₇ to R₁, R₂, R₃, R₄, R₅, R₆ and R₇ respectively as required, to obtain a compound of formula (I);

(ii) converting a compound of formula (I) into another compound of formula (I); and

(iii) preparing a salt of the compound of formula (I) and/or a solvate thereof.

Protected forms of R₄ will vary according to the particular nature of the group being protected but will be chosen in accordance with normal chemical practice.

Groups convertible to R₄ include groups dictated by conventional chemical practice to be required and to be appropriate, depending upon the specific nature of the R₄ under consideration.

Suitable deprotection methods for deprotecting protected forms of R₄ and conversion methods for converting R′₄ to R₄ will be those used conventionally in the art depending upon the particular groups under consideration with reference to standard texts such as Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994 and Chemistry of the Amino Group, Patais (Ed.), Interscience, New York 1968; or Advanced Organic Chemistry, March J, John Wiley & Sons, New York, 1992.

Suitable groups convertible into other groups include protected forms of said groups.

Advantageously, a compound of formula (XVII) will be a compound of formula (V) as defined above.

Suitably R′₁, R′₂, R′₃, R′₄, R′₅, R′₆ and R′₇ each represents R₁, R₂, R₃, R₄, R₅, R₆ and R₇ respectively or a protected form thereof.

Suitable deprotection methods for deprotecting protected forms of R₁, R₂, R₃, R₄, R₅, R₆ and R₇ and conversion methods for converting R′₁, R′₂, R′₃, R′₄, R′₅, R′₆ and R′₇ to R₁, R₂, R₃, R₄, R₅, R₆ and R₇ respectively will be those used conventionally in the art depending upon the particular groups under consideration with reference to standard texts such as Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994 and Chemistry of the Amino Group, Patais (Ed.), Interscience, New York 1968; or Advanced Organic Chemistry, March J, John Wiley & Sons, New York, 1992.

Suitably, reaction between the compounds of formulae (XVI) and (XVII) is carried out under conventional amination conditions, for example when L₁ is a bromine atom then the reaction is conveniently carried out in an aprotic solvent, such as tetrahydrofuran or dimethylformamide at any temperature providing a suitable rate of formation of the required product, usually at ambient temperature; preferably the reaction is carried out in the presence of triethylamine (TEA) or K₂CO₃.

The compounds of formula (XVII) are known, commercially available compounds or they can be prepared using methods analogous to those used to prepare known compounds; for example the methods described in the Chemistry of the Amino Group, Patais (Ed.), Interscience, New York 1968; Advanced Organic Chemistry, March J, John Wiley & Sons, New York, 1992 ; J. Heterocyclic Chem. (1990), 27, 1559; Synthesis (1975), 135, Bioorg. Med. Chem. Lett. (1997), 7, 555, or Protective Groups in Organic Synthesis (second edition), Wiley Interscience, (1991) or other methods mentioned herein.

A compound of formula (XVI) is prepared by appropriate halogenation of a compound of formula (XVIII):

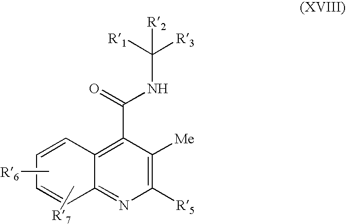

wherein R′₁, R′₂, R′₃, R′₅, R′₆, and R′₇ are as defined above in relation to formula (XVI).

Suitable halogenation reagents are conventional reagents depending upon the nature of the halogen atom required, for example when L₁ is bromine a preferred halogenation reagent is N-bromosuccinimide (NBS).

The halogenation of the compound of formula (XVIII) is carried out under conventional conditions, for example bromination is carried out by treatment with NBS in an inert solvent, such as carbon tetrachloride CCl₄, or 1,2-dichloroethane or CH₃CN, at any temperature providing a suitable rate of formation of the required product, suitably at an elevated temperature such as a temperature in the range of 60° C. to 100° C., for example 80° C.; preferably the reaction is carried out in the presence of a catalytic amount of benzoyl peroxide.

Suitably, the compound of formula (XVIII) may be prepared by reacting a compound of formula (VI) as defined above or an active derivative thereof with a compound of formula (III) as defined above wherein R′₂ is not aromatic in character.

It is favoured if the compound of formula (VI) is present in the reaction mix as an active derivative, as hereinbefore described.

The reaction between the compound of formula (VI) or the active derivative thereof and the compound of formula (III) is carried out under the appropriate conventional conditions for the particular compounds chosen. Generally, when the compound of formula (VI) is present as an active derivative the reaction is carried out using the same solvent and conditions as used to prepare the active derivative, preferably the active derivative is prepared in situ prior to forming the compound of formula (XVIII).

For example, the reaction between an active derivative of the compound of formula (VI) and the compound of formula (III) may be carried out:

(a) by first preparing an acid chloride and then coupling said chloride with the compound of formula (III) in the presence of an inorganic or organic base in a suitable aprotic solvent such as dimethylformamide (DMF) at a temperature in a range from −70 to 50° C. (preferably in a range from −10 to 20° C.); or

(b) by treating the compound of formula (VI) with a compound of formula (III) in the presence of a suitable condensing agent, such as for example N,N′-carbonyl diimidazole (CDI) or a carbodiimide such as dicyclohexylcarbodiimide (DCC) or N-dimethylaminopropyl-N′-ethylcarbodiimide, preferably in the presence of N-hydroxybenzotriazole (HOBT) to maximise yields and avoid racemization processes (see Synthesis, 453, 1972), or O-benzotriazol-1-yl-N,N,N′,N′-tetramethyluroniumhexafluorophosphate (HBTU), in an aprotic solvent, such as a mixture of acetonitrile (MeCN) and tetrahydrofuran (THF), for example a mixture in a volume ratio of from 1:9 to 7:3 (MeCN:THF), at any temperature providing a suitable rate of formation of the required product, such as a temperature in the range of from −70 to 50° C., preferably in a range of from −10 to 25° C., for example at 0° C.

A preferred reaction is set out in Scheme 2 shown below:

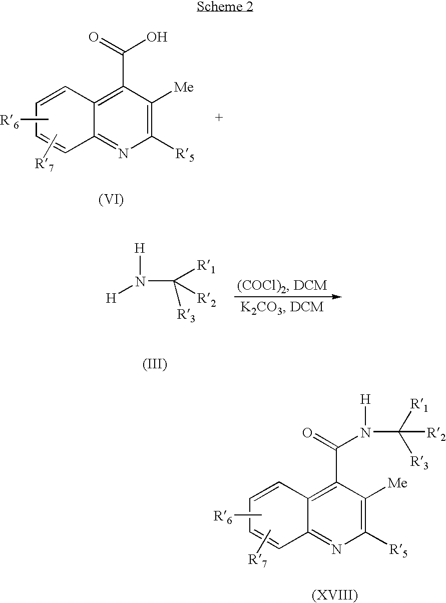

In the case in which the corresponding alkyl (such as methyl or ethyl) ester of compounds (VI) is utilised, a hydrolysis is required before conversion to compound (XVIII) in Scheme 2. Such hydrolysis can be carried out under acidic conditions, such 10-36% hydrochloric acid at a temperature in the range between 30 and 100° C.

In yet further embodiments, compounds of formula (Ib) can be prepared by reacting a compound of formula XIX

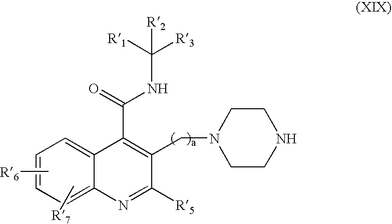

wherein R′₁, R′₂, R′₃, R′₅, R′₆, R′₇ and a are as defined above, with a compound of formula (XX)

L₃—R′₄ (XX)

wherein L₃ represents a leaving group for example halogen or activated ester, preferably chlorine, bromine or p-nitrophenylester and R′₄ represents R₄ as defined in relation to formula (I) or a protected form thereof or a group convertible thereto.

Compounds of formula (XIX) are prepared by removing the protective group of a compound of formula (XXII)

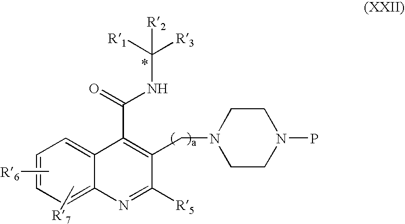

wherein R′₁, R′₂, R′₃, R′₅, R′₆, R′₇, and a are as defined above and P is an amine protective group, for example fmoc or benzyl, preferably fmoc. The protective group is removed by standard methods described in the literature, for example the fmoc residue is splitted by action of piperidine at room temperature in a solvent like acetonitrile.

As hereinbefore mentioned, the compounds of formula (I) may exist in more than one stereoisomeric form—and the process of the invention may produce racemates as well as enantiomerically pure forms. Accordingly, a pure enantiomer of a compound of formula (I) can be obtained by reacting a compound of the above defined formula (II) with an appropriate enantiomerically pure primary amine of formula (IIIa) or (IIIc):

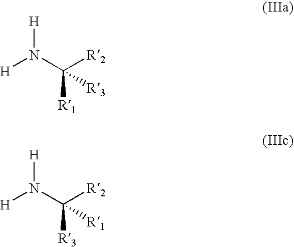

wherein R′₁, R′₂ and R′₃ are as defined above, to obtain a compound of formula (I′a) or (I′c):

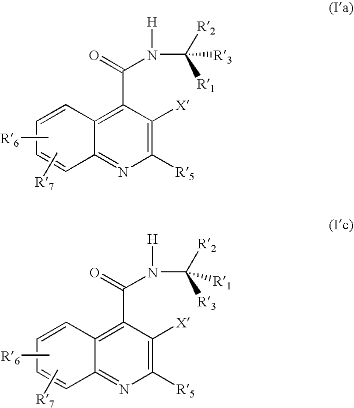

wherein R′₁, R′₂, R′₃, X′, R′₅, R′₆, and R′₇ are as defined above.

Compounds of formula (I′a) or (I′c) may subsequently be converted to compounds of formula (Ia) or (Ic) by the methods of conversion mentioned before:

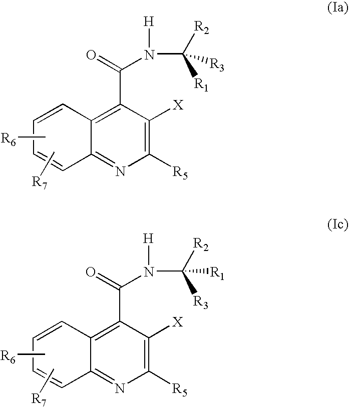

wherein R₁, R₂, R₃, X, R₅, R₆, and R₇ are as defined above.

Suitably, in the above mentioned compounds of formulae (Ia), (Ic), (I′a), (I′c), (IIIa) and (IIIc) R₁ represents hydrogen.

An alternative method for separating optical isomers is to use conventional, fractional separation methods in particular fractional crystallization methods. Thus, a pure enantiomer of a compound of formula (I) is obtained by fractional crystallisation of a diastereomeric salt formed by reaction of the racemic compound of formula (I) with an optically active strong acid resolving agent, such as camphosulphonic acid, tartaric acid, O,O′-di-p-toluoyltartaric acid or mandelic acid, in an appropriate alcoholic solvent, such as ethanol or methanol, or in a ketonic solvent, such as acetone. The salt formation process should be conducted at a temperature between 20° C. and 80° C., preferably at 50° C.

A suitable conversion of one compound of formula (I) into a further compound of formula (I) involves converting one group X into another group X by for example:

(i) converting a ketal into a ketone, by such as mild acidic hydrolysis, using for example dilute hydrochloric acid;

(ii) reducing a ketone to a hydroxy group by use of a borohydride reducing agent;

(iii) converting a carboxylic ester group into a carboxyl group using basic hydrolysis; and/or

(iv) reducing a carboxylic ester group to a hydroxymethyl group, by use of a borohydride reducing agent.

As indicated above, where necessary, the conversion of any group R′₁, R′₂, R′₃, X′, R′₅, R′₆, and R′₇ into R₁, R₂, R₃, X, R₅, R₆, and R₇ which as stated above are usually protected forms of R₁, R₂, R₃, X, R₅, R₆, or R₇ may be carried out using appropriate conventional conditions such as the appropriate deprotection procedure.

It will be appreciated that in any of the above mentioned reactions any reactive group in the substrate molecule may be protected and deprotected according to conventional chemical practice, for example as described by Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994.

Suitable protecting groups in any of the above mentioned reactions are those used conventionally in the art. Thus, for example suitable hydroxy protecting groups include benzyl or trialkylsilyl groups.

The methods of formation and removal of such protecting groups are those conventional methods appropriate to the molecule being protected. Thus for example a benzyloxy group may be prepared by treatment of the appropriate compound with a benzyl halide, such as benzyl bromide, and thereafter, if required, the benzyl group may be conveniently removed using catalytic hydrogenation or a mild ether cleavage reagent such as trimethylsilyl iodide or boron tribromide.

As indicated above, the compounds of formula (I) have useful pharmaceutical properties.

Accordingly the present invention also provides a compound of formula (I), or a pharmaceutically acceptable salt or solvate thereof, for use as an active therapeutic substance.

In particular, the present invention also provides a compound of formula (I), or a pharmaceutically acceptable salt or solvate thereof, for the treatment or prophylaxis of the Primary and Secondary Conditions.

The present invention further provides a pharmaceutical composition comprising a compound of formula (I), or a pharmaceutically acceptable salt or solvate thereof, and a pharmaceutically acceptable carrier.

The present invention also provides the use of a compound of formula (I), or a pharmaceutically acceptable salt or solvate thereof, in the manufacture of a medicament for the treatment of the Primary and Secondary Conditions.

As mentioned above the Primary conditions include respiratory diseases, such as chronic obstructive pulmonary disease (COPD), asthma, airway hyperreactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease, irritable bowel syndrome (IBS), gastro-exophageous reflex disease (GERD); urinary incontinence and disorders of the bladder function; renal disorders; increased blood pressure, proteinuria, coagulopathy and peripheral and cerebral oedema following pre-eclampsia in pregnancies.

As mentioned above, the Secondary conditions include disorders of the central nervous system such as anxiety, depression, psychosis and schizophrenia; neurodegenerative disorders such as AIDS related dementia, senile dementia of the Alzheimer type, Alzheimer's disease, Down's syndrome, Huntingdon's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilatation and vasospastic diseases such as angina, migraine and Reynaud's disease and pain or nociception, for example, that is attributable to or associated with any of the foregoing conditions especially the transmission of pain in migraine.

Such a medicament, and a composition of this invention, may be prepared by admixture of a compound of the invention with an appropriate carrier. It may contain a diluent, binder, filler, disintegrant, flavouring agent, colouring agent, lubricant or preservative in conventional manner.

These conventional excipients may be employed for example as in the preparation of compositions of known agents for treating the conditions.

Preferably, a pharmaceutical composition of the invention is in unit dosage form and in a form adapted for use in the medical or veterinarial fields. For example, such preparations may be in a pack form accompanied by written or printed instructions for use as an agent in the treatment of the conditions.

The suitable dosage range for the compounds of the invention depends on the compound to be employed and on the condition of the patient. It will also depend, inter alia, upon the relation of potency to absorbability and the frequency and route of administration.

The compound or composition of the invention may be formulated for administration by any route, and is preferably in unit dosage form or in a form that a human patient may administer to himself in a single dosage. Advantageously, the composition is suitable for oral, rectal, topical, parenteral, intravenous or intramuscular administration. Preparations may be designed to give slow release of the active ingredient.

Compositions may, for example, be in the form of tablets, capsules, sachets, vials, powders, granules, lozenges, reconstitutable powders, or liquid preparations, for example solutions or suspensions, or suppositories.

The compositions, for example those suitable for oral administration, may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinyl-pyrrolidone, sodium starch glycollate or microcrystalline cellulose; or pharmaceutically acceptable setting agents such as sodium lauryl sulphate.

Solid compositions may be obtained by conventional methods of blending, filling, tabletting or the like. Repeated blending operations may be used to distribute the active agent throughout those compositions employing large quantities of fillers. When the composition is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The composition may also be in the form of an ingestible capsule, for example of gelatine containing the compound, if desired with a carrier or other excipients.

Compositions for oral administration as liquids may be in the form of, for example, emulsions, syrups, or elixirs, or may be presented as a dry product for reconstitution with water or other suitable vehicle before use. Such liquid compositions may contain conventional additives such as suspending agents, for example sorbitol, syrup, methyl cellulose, gelatine, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

The compounds of this invention may also be administered by a non-oral route. In accordance with routine pharmaceutical procedure, the compositions may be formulated, for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi- dose forms such as a bottle from which the appropriate dose may be withdrawn or a solid form or concentrate which can be used to prepare an injectable formulation.

The compounds of this invention may also be administered by inhalation, via the nasal or oral routes. Such administration can be carried out with a spray formulation comprising a compound of the invention and a suitable carrier, optionally suspended in, for example, a hydrocarbon propellant.

Preferred spray formulations comprise micronised compound particles in combination with a surfactant, solvent or a dispersing agent to prevent the sedimentation of suspended particles. Preferably, the compound particle size is from about 2 to 10 microns.

A further mode of administration of the compounds of the invention comprises transdermal delivery utilising a skin-patch formulation. A preferred formulation comprises a compound of the invention dispersed in a pressure sensitive adhesive which adheres to the skin, thereby permitting the compound to diffuse from the adhesive through the skin for delivery to the patient. For a constant rate of percutaneous absorption, pressure sensitive adhesives known in the art such as natural rubber or silicone can be used.

As mentioned above, the effective dose of compound depends on the particular compound employed, the condition of the patient and on the frequency and route of administration. A unit dose will generally contain from 20 to 1000 mg and preferably will contain from 30 to 500 mg, in particular 50, 100, 150, 200, 250, 300, 350, 400, 450, or 500 mg. The composition may be administered once or more times a day for example 2, 3 or 4 times daily, and the total daily dose for a 70 kg adult will normally be in the range 100 to 3000 mg. Alternatively the unit dose will contain from 2 to 20 mg of active ingredient and be administered in multiples, if desired, to give the preceding daily dose.

No unacceptable toxicological effects are expected with compounds of the invention when administered in accordance with the invention.

The present invention also provides a method for the treatment and/or prophylaxis of the Primary and Secondary Conditions in mammals, particularly humans, which comprises administering to the mammal in need of such treatment and/or prophylaxis an effective, non-toxic pharmaceutically acceptable amount of a compound of formula (I) or a pharmaceutically acceptable salt or solvate thereof.

The activity of the compounds of the present invention, as NK₃ ligands, is determined by their ability to inhibit the binding of the radiolabelled NK₃ ligands, [¹²⁵I]-[Me-Phe⁷]-NKB or [³H]-Senktide, to guinea-pig and human NK₃ receptors (Renzetti et al, 1991, Neuropeptide, 18, 104-114; Buell et al, 1992, FEBS, 299(1), 90-95; Chung et al, 1994, Biochem. Biophys. Res. Commun., 198(3), 967-972).

The binding assays utilised allow the determination of the concentration of the individual compound required to reduce by 50% the [¹²⁵I]-[Me-Phe⁷]-NKB and [3H]-Senktide specific binding to NK₃ receptor in equilibrium conditions (IC50).

Binding assays provide for each compound tested a mean IC₅₀ value of 2-5 separate experiments performed in duplicate or triplicate. The most potent compounds of the present invention show IC₅₀ values in the range 0.1-1000 nM. The NK₃-antagonist activity of the compounds of the present invention is determined by their ability to inhibit senktide-induced contraction of the guinea-pig ileum (Maggi et al, 1990, Br. J Pharmacol., 101, 996-1000) and rabbit isolated iris sphincter muscle (Hall et al., 1991, Eur. J Pharmacol, 199, 9-14) and human NK₃ receptors-mediated Ca⁺⁺ mobilisation (Mochizuki et al, 1994, J Biol. Chem., 269, 9651-9658). Guinea-pig and rabbit in-vitro functional assays provide for each compound tested a mean KB value of 3-8 separate experiments, where K_{B} is the concentration of the individual compound required to produce a 2-fold rightward shift in the concentration-response curve of senktide. Human receptor functional assay allows the determination of the concentration of the individual compound required to reduce by 50% (IC₅₀ values) the Ca⁺⁺ mobilisation induced by the agonist NKB. In this assay, the compounds of the present invention behave as antagonists.

The activity of the compounds of the present invention, as NK-2 ligands, is determined by their ability to inhibit the binding of the radiolabelled NK-2 ligands, [¹²⁵I]-NKA or [³H]-NKA, to human NK-2 receptors (Aharony et al, 1992, Neuropeptide, 23, 121-130).

The binding assays utilised allow the determination of the concentration of the individual compound required to reduce by 50% the [¹²⁵I]-NKA and [³H]-NKA specific binding to NK2 receptor in equilibrium conditions (IC₅₀).

Binding assays provide for each compound tested a mean IC₅₀ value of 2-5 separate experiments performed in duplicate or triplicate. The most potent compounds of the present invention show IC₅₀ values in the range 0.5-1000 nM, such as 1-1000 nM. The NK-2-antagonist activity of the compounds of the present invention is determined by their ability to inhibit human NK-2 receptor-mediated Ca⁺⁺ mobilisation (Mochizuki et al, 1994, J. Biol. Chem., 269, 9651-9658). Human receptor functional assay allows the determination of the concentration of the individual compound required to reduce by 50% (IC₅₀ values) the Ca⁺⁺ mobilisation induced by the agonist NKA. In this assay, the compounds of the present invention behave as antagonists.

The therapeutic potential of the compounds of the present invention in treating the conditions can be assessed using rodent disease models.

As stated above, the compounds of formula (I) are also considered to be useful as diagnostic tools. Accordingly, the invention includes a compound of formula (I) for use as diagnostic tools for assessing the degree to which neurokinin-3 and neurokinin-2 receptor activity (normal, overactivity or underactivity) is implicated in a patient's symptoms. Such use comprises the use of a compound of formula (I) as an antagonist of said activity, for example including but not restricted to Tachykinin agonist-induced inositol phosphate turnover or electrophysiological activation, of a cell sample obtained from a patient. Comparison of such activity in the presence or absence of a compound of formula (I), will disclose the degree of NK-3 and NK-2 receptor involvement in the mediation of agonist effects in that tissue.

The following Descriptions illustrate the preparation of the intermediates, whereas the following Examples illustrate the preparation of the compounds of the invention.

### DESCRIPTIONS AND EXAMPLES

### Description 1

3-Methyl-2-phenyl-quinoline-4-carboxylic Acid Methyl Ester

4-Carboxy-3-methyl-2-phenylquinoline (48.5 g, 0.184 moles) (CAS [43071-45-0]) was suspended in DCM (500 ml) and oxalyl chloride (32.1 ml, 0.368 moles) was added dropwise at R.T. under magnetic stirring. After 15 min 2 drops of DMF were added. The reaction was vigorous with gas evolution. The mixture was stirred until the solid was completely dissolved (about 30 min.). The solution was evaporated and the oxalyl chloride excess was removed dissolving in DCM and evaporating the residue several time. The crude material was redissolved in DCM (250 ml) and quickly dropped into a solution of MeOH (500 ml) in DCM (250 ml). The dark and clear solution was let stand overnight and then evaporated to dryness obtaining a light coloured solid. Ethyl acetate and NaHCO₃ saturated solution was added and the mixture was stirred until the solid was completely dissolved. The layers were separated, the organic layer was washed twice with NaHCO₃, once with brine and then dried over Na₂SO₄, filtered and evaporated. The residue was cristallized from diethyl ether yielding 34 g of dark crystals that were in the next step used whithout further purification.

C₁₈H₁₅NO₂

MW=277.32

### Description 2

3-Bromomethyl-2-phenyl-quinoline-4-carboxylic Acid Methyl Ester

3-Methyl-2-phenyl-quinoline-4-carboxylic acid methyl ester (7.2 g, 26 mmoles), prepared as in Description 1, and NBS (9.2 g, 52 mmoles) were dissolved in CH₃CN (200 ml) and warmed to incipient reflux. Dibenzoyl peroxide (about 1 g) was carefully added portionwise and the solution was then refluxed for 4 h. The solution was evaporated to dryness, dissolved in ethyl acetate, washed with water, dried with Na₂SO₄, filtered and evaporated. The dark oil residue was purified by flash chromatography (eluent Hexane/Ethyl acetate=8/2) yelding after evaporation 7.3 g of dark oil wich solidified on standing.

C₁₈H₁₄BrNO₂

MW=356.23

### Description 3

Piperazine-1-carboxylic Acid Tert-butyl Ester

To a solution of piperazine (30 g, 350 mmol) in water (370 ml) and tBuOH (420 ml), a solution of 4N NaOH (70 ml) was added. The mixture was cooled to 0° C. and then BOC₂O (38 g, 170 mmol) was added portionwise. After stirring at room temperature for 45 minutes, tBuOH was evaporated under vacuum, the precipitate (diBOCpiperazine) was filtered and water was extracted with CH₂Cl₂. After drying over Na₂SO₄ the solvent was removed under vacuum to afford the title compound as a white solid (17g, 91 mmol). Yield: 54%

C₉H₁₈N₂O₂

MW=186.25

### Description 4

3-(4-tert-Butoxycarbonyl-piperazin-1-ylmethyl)-2-phenyl-quin line-4-carboxylic Acid Methyl Ester.

A solution of 3-bromomethyl-2-phenyl-quinoline-4-carboxylic acid methyl ester (8.2 g, 23 mmol), prepared as in Description 2, piperazine-1-carboxylic acid tert-butyl ester (4.7 g, 25 mmol), prepared as in Description 3, and DIEA (diisopropylethylamine, 8.5 ml, 49 mmol) in ThF (200 ml) was stirred at room temperature for 66 hours. The solvent was evaporated in vacuum, the residue was then re-dissolved in ethyl acetate, washed with a saturated solution of aqueous citric acid and the organic phase dried over Na₂SO₄. After concentration of the solvent the residue (10 g) was directly used for the next step without further purification.

C₂₇H₃₁N₃O₄

MW=461.56

### Description 5

3-(4-tert-Butoxycarbonyl-piperazin-1-ylmethyl)-2-phenyl-quinoline-4-carboxylic Acid.

A solution of 85% KOH (12.1 g, 184 mmol) in of n-PrOH (200 ml) and 3-(4-tert-butoxycarbonyl-piperazin-1-ylmethyl)-2-phenyl-quinoline-4-carboxylic acid methyl ester (10 g, 22 mmol), prepared as in Description 4, was heated to reflux, then 2 ml of water was added. Refluxing was continued for 16 hours. The mixture was concentrated to dryness in vacuum, water (100 ml) was added and washed with diethyl ether (3×50 ml), the aqueous layer was acidified with a saturated solution of citric acid (pH 6) and then extracted with ethyl acetate. The organic layer was washed with H₂O and dried over Na₂SO₄, filtered and evaporated to dryness to give 9.4 g (21 mmol) of the title compound. Yield 95%.

C₂₆H₂₉N₃O₄

MW=447.53

### Description 6

7-Methyl-6-phenyl-[1,3]dioxolo[4,5-g]quinoline-8-carboxylic acid

3,4-Methylenedioxyaniline (20.16 g, 147 mmol) was dissolved in EtOH (300 ml) and both benzaldehyde (14.3 ml, 147 mmol) and 2-oxobutirric acid (15 g, 147 mmol) were added. The solution was stirred at room temperature for three days. A solid was formed which was collected by filtration and dissolved in NaOH 1 M. The solution was washed with Et₂O and acidification of the aqueous phase a solid precipitated. The solid was filtered by suction and dried in vacuum oven to yield the title compound (25 g) as a pale brown solid.

C₁₈H₁₃NO₄

MW=307.30

MP=>300° C.

### Description 7

7-Methyl-6-phenyl-[1,3]dioxolo[4,5-g]quinoline-8-carboxylic Acid ((S)-1-cyclohexyl-ethyl)-amide

7-Methyl-6-phenyl-[1,3]dioxolo[4,5-g]quinoline-8-carboxylic acid (10 g, 32.5 mmol), prepared as in Description 6, was suspended in CH₂Cl₂ (200 ml) and cooled to 0-5° C. Oxalyl chloride (5.8 ml, 65 mmol) was added dropwise under stirring in 15 min. After adding few drops of DMF, the mixture was allowed to warm to room temperatureand left for 3 h. The organic solvent was evaporated to dryness. The crude residue was dissolved with CH₂Cl₂ and added dropwise to a stirred suspension of (S)-1-cyclohexylethylamine (5.8 ml, 39.05 mmol) and K₂CO₃ (9 g) in CH₂Cl₂ (150 ml). The solid was filtered and the organic solvent was evaporated to dryness. The crude residue was purified by flash chromatography (eluent hexane:AcOEt 6:4) obtaining 2.8 g of the title compound as a yellow solid.

C₂₆H₂₈N₂O₃

MW=416.52

### Description 8

4-[9-((S)-1-Cyclohexyl-ethylcarbamoyl)-7-phenyl-2,3-dihydro-[1,4]dioxino[2,3-g]quinolin-8-ylmethyl]-piperazine-1-carboxylic Acid Tert-butylester

7-Methyl-6-phenyl-[1,3]dioxolo[4,5-g]quinoline-8-carboxylic acid ((S)-1-cyclohexyl-ethyl)-amide (1.5 g, 3.5 mmol), prepared as in Description 7, and N-bromosuccinimmide (1.26 g, 7 mmol) were suspended in CCl₄ (60 ml) and warmed to incipient reflux. Dibenzoyl peroxide (about 10 mg) was carefully added and the solution was then refluxed for 2 h. The solvent was removed under vacuum and the residue was re-dissolved in CH₃CN (30 ml). This solution was added dropwise to a mixture of piperazine-1-carboxylic acid tert-butyl ester (1.3 g, 7 mmol ), prepared as in Description 3, and K₂CO₃ (1 g, 7 mmol) in CH₃CN (45 ml). The mixture was refluxed overnight. The organic solvent was evaporated to dryness, re-dissolved in AcOEt and washed with water, 10% citric acid and brine, dried over Na₂SO₄, filtered and concentrated. The crude residue was purified on column chromatography to yield 1.33 g of the title compound.

C₃₆H₄₆N₄O₅

MW=614.78

### Description 9

4-[4-((S)-1-Cyclohexyl-ethylcarbamoyl)-6,7-dimethoxy-2-phenyl-quinolin-3-ylmethyl]-piperazine-1-carboxylic Acid Tert-butyl Ester

This compound was prepared starting from 3,4-dimethoxyaniline and following the procedure described in Description 6-8.

### Description 10

3-(4-Fmoc-piperazin-1-ylmethyl)-2-phenyl-quinoline-4carboxylic Acid Methyl Ester

6.6 g (18.5 mmol) of crude 3-bromomethyl-2-phenyl-quinoline-4-carboxylic acid methyl ester (compound of Description 2) were dissolved, under nitrogen atmosphere, in 100 ml of dry THF. The solution was cooled to 10° C. and 6.8 g (20 mmol) of Fmoc piperazine, dissolved in 50 ml of THF, were added dropwise. The reaction mixture was allowed to warm to room temperature and stirred overnight. Salts were filtered off and the filtrate was evaporated in vacuo to dryness, taken up with 2 N HCl and washed with EtOAc; the aqueous layer was basified with 10% NaOH and extracted with CH₂Cl₂. The organic layer was dried over Na₂SO₄, filtered and evaporated in vacuo to dryness to obtain a crude material. Flash chromatography on silica gel afforded 7.5 g (yield: 69%) . of the title compound.

C₃₇H₃₃N₃O₄

MW=583.68

¹HNMR (DMSO-d₆) δ: 1.99 (4H); 3.10 (4H); 3.62 (2H); 3.97 (3H); 4.20 (1H); 4.42 (2H); 7.18-7.40 (4H ar); 7.45-7.92 (12H ar); 8.09 (1H ar)

### Description 11

3-(4-Fmoc-piperazin-1-ylmethyl)-2-phenyl-quinoline-4-carboxylic acid hydrochloride

7.5 g (13 mmol) of the ester of Description 10 were dissolved in 150 ml of 6 N HCl and refluxed for 1 h. Evaporation to dryness afforded 9.5 g of crude title compound, which was used in the following reaction without further purification.

C₃₆H₃₁N₃O₄.HCl

MW=606.12

H NMR (DMSO-d₆) δ:2.50 (4H); 3.32 (4H); 4.22 (2H); 4.23 (1H); 4.35 (2H); 6.50 (1H exch with D₂O); 7.22-7.88 (14H ar); 7.98 (1H ar); 8.17 (2H ar)

### Description 12

3-(4-Fmoc-piperazin-1-ylmethyl)-2-phenyl-quinoline-4-carboxylic Acid ((S)-1-cyclohexyl-ethyl)-amide

A mixture of 5 g (7.8 mmol) N-fmoc-2-phenyl-3-piperazin-1-ylmethyl-quinoline-4-carboxylic acid (compound of Description 11), 3.14 g (31 mmol) triethylamine, 4.44 g (11.7 mmol) HBTU, 100 ml anhydrous THF, 1.18 g (11.7 mmol) (S)-(+)-1-cyclohexylethylamine and 65 ml methylene chloride was stirred one night at room temperature. The mixture was concentrated in vacuo, the residue was dissolved in ethyl acetate and the organic phase washed with water. After drying over MgSO₄ the solvent was concentrated and the residue purified by flash chromatography over 350 g silicagel (eluent: first heptane/ethyl acetate: 2/1 then 1/1) to afford 4 g (yield 74%) of the title compound.

C₄₄H₄₆N₄O₃

MW=678.87

¹H-NMR (CDCl₃) δ: 0.70-1.95 (m, 14H); 1.98-2.25 (m, 4H); 2.95-3.42 (m, 4H); 3.75 (s, 2H); 4.17 (t, 1H); 4.28 (m, 1H); 4.38 (d, 2H); 6.95-7.80 (m, 16H); 8.05 (d, 1H ar); 8.15 (d, 1H ar)

### Description 13

3-(4-Fm c-piperazin-1-ylmethyl)-2-phenyl-quin line4-carb xylic Acid ((S)-2-methyl-1-phenyl-propyl)-amide

6.95 g (10.8 mmol) of crude acid of Description 11 were condensed with 2 g (13.5 mmol) of (S)-2-methyl- 1-phenyl propylamine following the procedure of Description 12 affording, after flash chromatography on silica gel, 5.4 g (yield 71%) of the title compound.

C₄₆H₄₄N₄O₃

MW=700.86

¹H NMR (CDCl₃)δ:0.96 (3H); 1.18 (3H); 1.56-2.98 (4H); 2.28 (1H); 3.04 (4H); 3.53 (2H); 4.20 (1H); 4.35 (2H); 5.17 (1H); 7.18-7.63 (18H ar); 7.74 (3H ar); 7.97 (1H exch with D₂O); 8.14 (1H ar)

### Description 14

3-(4-Fmoc-piperazin-1-ylmethyl)-2-phenyl-quinoline-4-carboxylic acid ((S)-1-phenyl-ethyl)-amide

Synthesised starting from the compound of Description 11 and following the procedure of Description 12.

C₄₄H₄₀N₄O₃

MW=672.83

### Description 15

2-Phenyl-3-piperazin-1-ylmethyl-quinoline4-carboxylic acid ((S)-1-cyclohexyl-ethyl)-amide

A mixture of 4 g (5.8 mmol) of N-fmoc-2-phenyl-3-piperazin-1-ylmethyl-quinoline-4-carboxylic acid ((S)-1-cyclohexyl-ethyl)-amide (compound of Description 12),160 ml acetonitrile and 890 microliters (9 mmol) piperidine was stirred one night at room temperature. The solvent was concentrated and the residue purified by flash chromatography on 150 g silicagel (eluent: first CH₂Cl₂/MeOH: 9/1 then CH₂Cl₂/MeOH/NH₄OH: 9/1/0.1) to afford 1.86 g (70.2%) of the title compound.

C₂₉H₃₆N₄O

MW=456.63

¹H-NMR (CDCl₃)δ: 0.85-1.55 (m, 9H); 1.56-1.98 (m, 5H); 2.00-2.25 (m, 4H); 2.50-2.85 (m, 4H); 3.73 (s, 2H); 4.24 (m, 1H); 7.28-7.78 (7H ar); 7.80-8.19 (4H)

### Description 16

2-Phenyl-3-piperazin-1-ylmethyl-quinoline4-carboxylic Acid ((S)-2-methyl-1-phenyl-propyl)-amide

5.4 g (7.7 mmol) of the Fmoc derivative of Description 14 were reacted with 1.25 ml of piperidine in 200 ml acetonitrile, at room temperature for one night. The reaction mixture was concentrated to dryness and the residue was purified by flash chromatography on silicagel (eluent: CH₂Cl₂/CH₃OH/NH₄OH; 90/10/2), affording 2.55 g (yield 69.3%) of the title compound.

C₃₁H₃₄N4O

MW=478.64

¹H NMR (DMSO-d₆)δ: 0.79 (3H); 1.06 (3H); 1.49-2.55 (9H); 3.45 (2H and 1H exch with D₂O); 4.88 (1H); 7.12-8.10 (14H ar); 9.16 (1H exch with D₂O)

### Description 17

2-Phenyl-3-piperazin-1-ylmethyl-quinoline4-carboxylic acid ((S)-1-phenyl-ethyl)-amide

Synthesised starting from the compound of Description 14 and following the procedure of Description 15.

C₂₉H₃₀N₄O

MW=450.58

### Description 18

3-[4-(1-Methylsulfanyl-2-nitro-vinyl)-piperazin-1-ylmethyl]-2-phenyl-quinoline-4-carboxylic Acid ((S)-1-phenyl-ethyl)-amide

A solution of 0.39 g (0.865 mmol) of 2-phenyl-3-piperazin-1-ylmethyl-quinoline-4-carboxylic acid ((S)-1-phenyl-ethyl)-amide (compound of Description 17) and 0.14 g (0.865 mmol) of 1,1-bis-methylsulfanyl-2-nitro-ethene in a mixture of 7.5 ml of ethanol and 1.8 ml of DMF was heated to reflux for 15 h. The solvents were concentrated and the residue was purified by flash chromatography on silicagel (eluent: heptane/AcOEt: 1/1) to afford 0.13 g of the title compound as yellow crystals.

C₃₂H₃₃N₅O₃S

MW=567.71

¹H-NMR (CDCl₃)δ: 1.72 (d, 3H); 1.85-2.21 (m, 4H); 2.34 (s, 3H); 2.87-3.22 (m, 4H); 3.66 (s, 2H); 5.55 (m, 1H); 6.52 (s, 1H); 7.19-7.67 (m, 12H); 7.78 (td, 1H ar); 8.00 (d,1H ar); 8.14 (dd, 1H ar)

### Description 19

3-[4-(1-Methylsulfanyl-2-nitro-vinyl)-piperazin-1-ylmethyl]-2-phenyl-quinoline-4-carboxylic acid ((S)-2-methyl-1-phenyl-propyl)-amide

Starting from 2-phenyl-3-piperazin- 1-ylmethyl-quinoline4-carboxylic acid ((S)-2-methyl-1-phenyl-propyl)-amide (compound of Description 16) and following the procedure of Description 18 afforded the title compound.

C₃₄H₃₇N₅O₃S

MW=580.73

¹H-NMR (CDCl₃)δ: 0.94 (d, 3H); 1.22 (d, 3H); 1.74-2.18 (m, 4H); 2.20 (m, 1H); 2.35 (s, 3H); 2.98-3.27 (m, 4H); 3.52 (s, 2H); 5.13 (m, 1H); 6.53 (s, 1H); 6.85 (d, 1H ar); 7.15-7.65 (m, 11H); 7.74 (t,1H ar); 7.92 (br, 1H); 8.14 (d, 1H)

### Description 20

2-Benzyl-3-{4-[4-((S)-1-cyclohexyl-ethylcarbamoyl)-2-phenyl-quinolin-3-ylmethyl]-piperazin-1-yl}-propionic acid ethyl ester (racemic)

A solution of 0.40 g (0.87 mmol) 2-phenyl-3-piperazin-1-ylmethyl-quinoline-4-carboxylic acid ((S)-1-cyclohexyl-ethyl)-amide (compound of Description 15) and 0.33 g (1.75 mmol) of 2-phenyl-but-3-enoic acid ethyl ester in 10 ml isopropanol was heated to reflux for 48 h. A white suspension appeared but TLC monitoring proved the reaction to be not completed. Additional 100 mg of 2-phenyl-but-3-enoic acid ethyl ester were the added and the reflux continued for 4 h. The solvent was concentrated and the residue was purified by flash chromatography over silicagel (eluent: CH₂Cl₂/MeOH:95/5) affording 0.17 g of the title compound.

C₄₁H₅₀N₄O₃

MW=646.87

¹H-NMR (CDCl₃)δ:1.07 (t, 3H); 1.16 (m, 5H); 1.27 (d, 3H); 1.40 (m, 1H); 1.65-1.95 (m, 5H); 2.00-2.45 (8H); 2.50-2.96 (m, 5H); 3.72 (2H); 3.99 (q, 2H); 4.27 (m, 1H); 7.02-7.30 (m, 5H ar); 7.46 (m, 5H ar); 7.58 (td, 1H ar); 7.73 (td, 1H ar); 8.05-8.18 (m, 2H ar); 8.22 (broad band, 1H)

### Description 21

3-{4-[4-((S)-2-Methyl-1-phenyl-propylcarbamoyl)-2-phenyl-quinolin-3-ylmethyl]-piperazin-1-yl}-2-phenyl-propionic acid ethyl ester

Following the procedure of Description 20 but starting from 2-phenyl-3-piperazin-1-ylmethyl-quinoline-4-carboxylic acid ((S)-2-methyl-1-phenyl-propyl)-amide (compound of Description 16) and using 2-phenyl-acrylic acid ethyl ester afforded the title compound. (reaction yield : 47%, conversion yield : 70%).

C₄₂H₄₆N₄O₃

MW=654.85

¹H-NMR (CDCl₃)δ: 0.94 (d, 3H); 1.07-1.22 (6H); 1.65-2.48 (m, 10H); 3.04 (t, 1H); 3.55 (s, 2H); 3.67 (m, 1H); 4.07 (m, 2H); 5.16 (m, 1H); 7.14-7.61 (m, 16H ar); 7.71 (td, 1H ar); 8.02 (d, 1H ar); 8.12 (dd, 1H ar); 8.50 (br, 1H)

### Description 22

3-{4-[4-((S)-1-Cyclohexyl-ethylcarbamoyl)-2-phenyl-quinolin-3-ylmethyl]-piperazin-1-yl}-propionic acid tert-butyl ester

The title compound was obtained starting from 2-phenyl-3-piperazin-1-ylmethyl-quinoline-4-carboxylic acid ((S)-1-cyclohexyl-ethyl)-amide (compound of Description 15) and t-butyl acrylate, following the procedure of description 21.

C₃₆H₄₈N₄O₃

MW=584.80

H¹-NMR (CDCl₃) δ:0.95-1.95 (m, 13H); 1.28 (d, 3H); 1.40 (s, 9H); 2.02-2.68 (m, 10H); 3.75 (s, 2H); 4.25 (m, 1H); 7.44 (m, 5H ar); 7.58 (td, 1H ar); 7.74 (td, 1H ar); 8.02-8.19 (m, 2H ar); 8.28 (br, 1H)

### Description 23

6-Fluoro-3-methyl-2-phenylquinoline4-carboxylic acid

To a solution of 5-fluoroisatin (3 g, 0.018 moles) (CAS [443-69-6]) in EtOH (100 ml), KOH in pellets (4.7 g, 0.08 moles) was added. The mixture was stirred for 30 minutes at room temperature, then 1-phenylpropan-1-one (CAS [93-55-0]) (2.4 g, 0.018 moles) was added and the solution was refluxed for additional 4 hours. The solvent was evaporated under vacuum and the residue was dissolved in water (200 ml), the water was extracted with ethyl ether (200 ml) and the aqueous solution was acidified with a saturated solution of citric acid. The obtained precipitate was filtered and dried in vacuum oven to yield the title compound (3 g) as a pale yellow solid. Yield

63%

C₁₇H₁₂FNO₂

MW=281.29

### Description 24

6-Fluoro-3-methyl-2-phenylquinoline-4-carboxylic acid ((S)-1-cyclohexylethyl)amide

6-Fluoro-3-methyl-2-phenylquinoline4-carboxylic acid (2 g, 7.6 mmol), prepared as in Description 23, was suspended in CH₂Cl₂ (20 ml) and cooled to 0-5° C. Oxalyl chloride (1.5 ml, 25 mmol) was added drop-wise under stirring in 15 min. After adding few drops of DMF, the mixture was allowed to warm to room temperature and left for 3 h. The organic solvent was evaporated to dryness. The crude residue was dissolved with CH₂Cl₂ and added drop-wise to a stirred suspension of (S)-1-cyclohexylethylamine (1.5 ml, 10 mmol) and K₂CO₃ (3 g) in CH₂Cl₂ (10 ml). The solution was refluxed for 3 hours then concentrated under vacuum. The residue was re-dissolved in AcOEt and the organic layer was washed with a solution of 1 M NaOH, with a saturated solution of NaCl and finally dried over Na₂SO₄, filtered and evaporated to dryness. The crude residue was triturated with diisopropylether obtaining 2.3 g of the title compound as a pale yellow solid. Yield 76%.

C₂₅H₂₇FN₂O

MW 390.51

### Description 25

3-Bromomethyl-6-fluoro-2-phenylquinoline4-carboxylic Acid ((S)-1-cyclohexylethyl)-amide 6-Fluoro-3-methyl-2-phenyl-quinoline-4-carboxylic acid ((S)-1-cyclohexylethyl)-amide (1.4 g, 0.003 moles), prepared as in Description 24, and NBS (1.3 g, 0.0076 moles) were dissolved in CCl₄ (50 ml) and warmed to incipient reflux. Dibenzoyl peroxide (about 1 g) was carefully added portion-wise and the solution was then refluxed for 2 h. The solution was evaporated to dryness, dissolved in ethyl acetate, washed with a 10% solution of Na₂CO₃, dried with Na₂SO₄, filtered and evaporated. The dark oil residue was purified by flash chromatography (eluent hexane/ethyl acetate=8/2) yielding after evaporation 1.3 g of a pale yellow solid. Yield 77%.

C₂₅H₂₆BrFN₂O

MW=469.40

### Description 26

4-[4-((S)-1-cyclohexylethylcarbamoyl)-6-fluoro-2-phenylquinolin-3-ylmethyl]-piperazine-1-carboxylic acid tert-butyl ester

A solution of: 3-bromomethyl-6-fluoro-2-phenylquinoline-4-carboxylic acid ((S)-1-cyclohexylethyl)amide (0.3 g, 0.6 mmol; compound prepared as in Description 25), piperazine-1-carboxylic acid tert-butyl ester (0.13 g, 0.7mmol) and ethyldiisopropylamine (0.3 ml, 1.8 mmol) in dry THF (30 ml) was stirred for 24 h at room temperature. The solvent was evaporated to dryness in vacuo and the residue was re-dissolved in EtOAc. This mixture was washed with a dilute NaOH solution, with water and dried over Na₂SO₄. After evaporating to dryness, the residue was purified by flash chromatography to afford 0.25 g of the desired compound. Yield 72%

C₃₄H₄₃FN₄O₃

MW=574.75

### Description 27

{Carboxymethyl-[4-((S)-1-cyclohexylethylcarbamoyl)-2-phenylquinolin-3-ylmethyl]amino}-acetic acid

A solution of 3-bromomethyl-2-phenylquinoline4-carboxylic acid ((S)-1-cyclohexylethyl)-amide (5 g, 11 mmol, prepared from isatine (CAS [91-56-5]) according to Description 23-25), (carboxymethylamino)acetic acid (2.2 g, 16 mmol) and ethyldiisopropylamine (14 ml, 80 mmol) in acetonitrile (100 ml) was stirred at room temperature for 12 hours. The solvent was evaporated under vacuum, a solution of 2N NaOH was added and the obtained precipitated was filtered. The organic layer was neutralized with 1N HCl and extracted with EtOAc.The organic layer was dried over Na₂SO₄, filtered and evaporated to give the title compound (4 g).Yield: 72%

C₂₉H₃₃N₃O₅

MW=503.60

### Description 28

4-[4-((S)-1-Cyclohexylethylcarbamoyl)-2-phenylquinolin-3-ylmethyl]-3-oxopiperazine-1-carboxylic acid tert-butyl ester

NaH (0.09 g, 3.4 mmol) was added portion-wise at room temperature to a suspension of 3-oxo-piperazine-1-carboxylic acid tert-butyl ester (0.6 g, 3 mmol, CAS [76003-29-7]) in DMF (10 ml) and DMSO (3 ml), . The obtained dark solution was stirred for 30 minutes then a solution of 3-bromomethyl-2-phenylquinoline-4-carboxylic acid ((S)-1-cyclohexylethyl)-amide (1.3 g, 2.8 mmol, prepared from isatine (CAS [91-56-5]) according to Description 23-25) in DMF (5 ml) was added. The mixture was stirred for additional 3 hours and then was poured in a saturated solution of NaCl. The obtained precipitate was filtered by suction and dried in vacuum oven to yield the title compound

(1 g, 1.7 mmol). Yield 63%

C₃₄H₄₂N₄O_{4 MW=}570.74

### Description 29

8-Methyl-7-phenyl-2,3-dihydro-[1,4]dioxino[2,3-g]quinoline-9-carboxylic acid

Benzaldehyde (6.7 ml, 66 mmol) was added drop-wise to a solution of 2,3-dihydrobenzo[1,4]dioxin-6-ylamine (10 g, 66 mmol) in EtOH (200 ml). The solution was refluxed for 1 hour and then 2-oxobutyric acid (6.7 g, 66 mmol) was added portion-wise. The mixture was refluxed for additional 3 hours and then left at room temperature overnight. The obtained precipitate was filtered to give 13 g of the title compound.

Yield 61%

C₁₉H₁₅NO₄

MW=321.34

### Description 30

8-Br momethyl-7-phenyl-2,3-dihydro-[1,4]di xino[2,3-g]quinoline-9-carboxylic acid Methyl Ester

The compound was prepared following the procedure of Description 1 and 2 starting from 8-methyl-7-phenyl-2,3-dihydro-[1,4]dioxino[2,3-g]quinoline-9-carboxylic acid (prepared as in Description 29).

C₂₀H₁₆BrNO₄

MW:414.26

### Description 31

8-(3-Oxo-piperazin- 1-ylmethyl)-7-phenyl-2,3-dihydro-[1,4]dioxino[2,3-g]quinoline-9-carboxylic Acid Methyl Ester

A solution of: 8-bromomethyl-7-phenyl-2,3-dihydro-[1,4]dioxino[2,3-g]quinoline-9-carboxylic acid methyl ester (414 mg, 1 mmol, prepared as in Description 30), piperazin-2-one (CAS [5625-67-2]) (0.1 g, 1 mmol) and ethyldiisopropylamine (0.3 ml, 1.8 mmol) in dry THF (30 ml) was stirred for 24 h at room temperature. The solvent was evaporated to dryness in vacuo and the residue was re-dissolved in AcOEt. This mixture was washed with a dilute NaOH solution, with water and dried over Na₂SO₄. After evaporating to dryness, the residue was purified by flash chromatography (eluent Ethyl acetate/ Methanol/ NH₃=90/10/0.1) to afford 0.3 g of the desired compound.

Yield 69%

C₂₄H₂₃N₃O₅

MW: 433.47

### Description 32

8-(3-Oxo-piperazin-1-ylmethyl)-7-phenyl-2,3-dihydro-[1,4]dioxino[2,3-g]quinoline-9-carboxylic Acid

A solution of 85% KOH (0.46 g, 7 mmol) and 8-(3-oxo-piperazin-1-ylmethyl)-7-phenyl-2,3-dihydro-[1,4]dioxino[2,3-g]quinoline-9-carboxylic acid methyl ester (0.5 g, 1.2 mmol, prepared as in Description 31), in MeOH (20 ml), was heated to reflux for 36 hours then citric acid (1.47 g, 7 mmol) was added. The inorganic salts were filtered and the MeOH was evaporated. The crude solid was triturated with ether to give 0.4 g of the title compound.

C₂₃H₂₁N₃O₅

MW=419.44

### GENERAL PROCEDURE FOR THE PREPARATION OF EXAMPLES 1-3, 5, 7, 8.

A solution of of 3-(4-tert-butoxycarbonyl-piperazin-1-ylmethyl)-2-phenyl-quinoline-4-carboxylic acid (2 g, 4.5 mmol), prepared as in Description 5, suitable amine (5.7 mmol), DCC (1.2 g, 5.8 mmol) and DMAP (0.7 g, 5.8 mmol) in of CH₂Cl₂ (60 ml) was stirred for 24 h at room temperature. The resultant solid was filtered and the filtrate was evaporated to dryness. The residue was re-dissolved in AcOEt, washed with a 10% NaCl solution and dried over MgSO₄. After concentration of the solvent, the crude product was dissolved in CH₂Cl₂ (60 ml) and TFA (3 ml) was added. The red solution was stirred at room temperature overnight; then the solvent and the excess of TFA were removed under vacuum. The residue was dissolved in H₂O and washed 2 times with Et₂O. The water extract was made alkaline by addition of 2N NaOH solution and the product was extracted with AcOEt. The solvent was evaporated to dryness and the residue was purified by flash chromatography (eluent CH₂Cl₂: MeOH 93:7) to afford the title compound (yield: 30-50%).

### Example 4

### 2-Phenyl-3-piperazin-1-ylmethyl-quinoline-4-carboxylic Acid [(S)-1-(3-hydroxy-phenyl)-ethyl]-amide

To a solution of 2-phenyl-3-piperazin- 1-ylmethyl-quinoline-4-carboxylic acid [(S)- 1-(3-methoxy-phenyl)-ethyl]-amide (300 mg, 0.62 mmol), compound of Example 3, in CH₂Cl₂ (20 ml), BBr₃ (0.020 ml, 0.31 mmol) of was added at 0° C. After stirring the solution overnight at room temperature, the solvent was removed under vacuum. The residue was redissolved in AcOEt and washed with Na₂CO₃20% solution. The organic layer was dried over Na₂SO₄ and evaporated to dryness. The residue was purified by flash chromatography (eluent: CH₂Cl₂/MeOH/NH₄OH 90:10:1) to afford the title compound.

### Example 6

### 2-Phenyl-3-piperazin-1-ylmethyl-quinoline-4-carboxylic Acid [(S)-1-(4-hydroxy-phenyl)-ethyl]-amide

The title compound was prepared starting from the Example 2 following the procedure of Example 4.

### Example 9

### 7-Phenyl-8-piperazin-1-ylmethyl-2,3-dihydro-[1,4]dioxino[2,3-g]quinoline-9-carboxylic Acid ((S)-1-cyclohexyl-ethyl)-amide

To a solution of 4-[9-((S)-1-Cyclohexyl-ethylcarbamoyl)-7-phenyl-2,3-dihydro-[1,4]dioxino[2,3-g]quinolin-8-ylmethyl]-piperazine-1-carboxylic acid tert-butylester (1.25 g, 2 mmol), prepared as in Description 8, in CH₂Cl₂ (50 ml), TFA (2 ml) was added dropwise at room temperature. Stirring was continued overnight. The solvent was evaporated under vacuum and the residue was basified K₂CO₃ saturated solution and extracted with ethyl acetate. The organic layer was dried over Na₂SO₄, filtered and evaporated. The residue was purified on column chromatography

(CH₂Cl₂/MeOH/NH₄OH 90:10:1) to give the title compound (0.45 g).

### Example 10

### 6,7-Dimethoxy-2-phenyl-3-piperazin-1-ylmethyl-quinoline-4-carboxylic Acid ((S)-1-cyclohexyl-ethyl)-amide

The title compound was prepared following the procedure of Example 9 starting from the compound described in Description 9.

### Example 11

### 2-{4-[4-((S)-2-Methyl-1-phenyl-propylcarbamoyl)-2-phenyl-quinolin-3-ylmethyl]-piperazin-1-yl}-ethanesulfonic Acid Phenyl Ester

A solution of 0.092 g (0.5 mmol) of phenyl vinylsulfonate in 2 ml of methylene chloride was cooled by an ice bath. Then 0.24 mg (0.5 mmol) of 2-phenyl-3-piperazin-1-ylmethyl-quinoline-4-carboxylic acid ((S)-2-methyl-1-phenyl-propyl)-amide (compound of Description 16) were added portionwise. Stirring was maintained 30 min at the temperature of the ice bath followed by 3 h at room temperature.

The solvent was concentrated and the residue was purified by flash chromatography on silicagel (eluent: AcOET/heptane: 1/1) to afford 200 mg (60.5%) of the title compound as a white amorphous solid.

### Example 12

### 3-[4-(2-Nitro-1-pyrrolidin-1-yl-vinyl)-piperazin-1-ylmethyl]-2-phenyl-quin line-4-carboxylic Acid ((S)-1-phenyl-ethyl)-amide

Starting form 3-[4-(1-methylsulfanyl-2-nitro-vinyl)-piperazin-1-ylmethyl]-2-phenyl-quinoline-carboxylic acid ((S)-1-phenyl-ethyl)-amide (compound of Description 18) and 1 g of pyrrolidine in 10 ml acetonitrile was refluxed for 4 h. The solvent was concentrated, the residue dissolved in ethyl acetate, the organic phase washed with water, dried over MgSO₄ and concentrated again. The residue was purified by flash chromatography on silicagel (eluent: first AcOEt, then AcOEt/MeOH:9/1). The residue obtained after concentration of the desired fractions was triturated in diethyl ether affording 75 mg (73%) of the title compound as yellow crystals.

### Example 13

### 3-[4-(2-Nitro-1-pyrrolidin-1-yl-vinyl)-piperazin-1-ylmethyl]-2-phenyl-quinoline-4-carboxylic Acid ((S)-2-methyl-1-phenyl-propyl)-amide

Starting from 3-[4-(1-methylsulfanyl-2-nitro-vinyl)-piperazin-1-ylmethyl]-2-phenyl-quinoline-4-carboxylic acid ((S)-2-methyl- 1-phenyl-propyl)-amide (compound of Description 19) and following the procedure of Example 12 afforded the title compound as orange crystals.

### Example 14

### 2-Methyl-3-{4-[2-phenyl-4-((S)-1-phenyl-ethylcarbamoyl)-quinolin-3-ylmethyl]-piperazin-1-yl}-propionic Acid.

A solution of 0.25 g (0.54 mmol) of 2-phenyl-3-piperazin-1-ylmethyl-quinoline-4-carboxylic acid ((S)-1-phenyl-ethyl)-amide (compound of Description 17) and 0.13 g (0.8 mmol) of trimethylsilyl methacrylate (Aldrich) in 5 ml dry chloroform was heated at 65° C. for 24 h. After cooling, 1 ml of methanol was added and the mixture stirred for 10 min. The solvent was concentrated and the residue was purified by flash chromatography on silicagel (eluent : first CH₂Cl₂/MeOH : 95/5; then CH₂Cl₂/MeOH: 90/10). The desired fractions were pooled, the solvent concentrated and the residue was crystallised in diethyl ether to afford the title compound as white crystals.

### Example 15

### 1-(2-Nitr -1-{4-[2-phenyl-4-((S)-1-phenyl-ethylcarbamoyl)-quin lin-3-ylmethyl]-piperazin-1-yl}-vinyl)-piperidine-3-carboxylic acid ethyl ester.

Starting form 3-[4-(1-methylsulfanyl-2-nitro-vinyl)-piperazin-1-ylmethyl]-2-phenyl-quinoline4-carboxylic acid ((S)-1-phenyl-ethyl)-amide (compound of Description 18) and following the procedure of Example 12 but replacing the pyrrolidine by ethyl nipecotate afforded the title compound as a yellow amorphous solid.

### Example 16

### 3-{4-[4-((S)-2-Methyl-1-phenyl-propylearbamoyl)-2-phenyl-quinolin-3-ylmethyl]-piperazin-1-yl}-2-phenyl-propionic Acid

A mixture of 0.26 g (0.4 mmol) of 3-{4-[4-((S)-2-methyl-1-phenyl-propylcarbamoyl)-2-phenyl-quinolin-3-ylmethyl]-piperazin-1-yl}-2-phenyl-propionic acid ethyl ester (compound of Description 21), 0.42 ml of 1N aqueous LiOH and 2.5 ml of ethanol was stirred at room temperature for 24 h, the 0.3 ml of LiOH solution were added again and stirring was continued for 6 h. 20 ml of AcOEt were added followed and the mixture was stirred with a saturated aqueous solution of KHSO₄. The organic phase was decanted and washed with water. The aqueous phase was extracted twice with CH₂Cl₂, the organic phases were pooled, dried over MgSO₄ and concentrated. The residue was purified twice by flash chromatography on silicagel (eluent: CH₂Cl₂/MeOH: 92/8) to afford 100 mg of the title compound as white crystals.

### Example 17

### 2-Benzyl-3-{4-[4-((S)-1-cyclohexyl-ethylcarbamoyl)-2-phenyl-quinolin-3-ylmethyl]-piperazin-1-yl}-propionic Acid (Racemic)

A solution of 0.16 g ( 0.25 mmol) of racemic 2-benzyl-3-{4-[4-((S)-1-cyclohexyl-ethylcarbamoyl)-2-phenyl-quinolin-3-ylmethyl]-piperazin-1-yl}-propionic acid ethyl ester (compound of Description 20) and 250 microliters of aqueous 1 N LiOH in 10 ml ethanol was stirred at room temperature for 48 h. Meanwhile 100 ml of 1 N LiOH were added twice. The solvent was concentrated and the residue taken-up in 15 ml CH₂Cl₂ and washed with an aqueous saturated solution of KHSO₄. After drying over MgSO₄ the solvent was concentrated and the residue (0.19 g) was purified by flash chromatography over silicagel (eluent: CH₂Cl₂/MeOH:95/5) affording 0.073 g of the title compound as white solid.

### Example 18

### 3-{4-[4-((S)-1-Cyclohexyl-ethylcarbamoyl)-2-phenyl-quinolin-3-ylmethyl]-piperazin-1-yl}-propionic Acid

A mixture of 0.38 g (6.4 mmol) of 3-{4-[4-((S)-1-cyclohexyl-ethylcarbamoyl)-2-phenyl-quinolin-3-ylmethyl]-piperazin- 1-yl}-propionic acid tert-butyl ester (compound of Description 22), 5 ml of methylene chloride and 5 ml of trifluoroacetic acid was stirred at room temperature for 4 h. The solvent was concentrated and the residue, after neutralisation with 1N aqueous NH₄OH, was purified by flash chromatography on silicagel (eluent: first CH₂Cl₂/MeOH:95/5, then 90/10) afforded the title compound as white crystals.

### Example 19

### 3-(4-Carbamoylmethyl-piperazin-1-ylmethyl)-2-phenyl-quinoline-4-carboxylic Acid ((S)-1-cyclohexyl-ethyl)-amide

A mixture of 0.5 g (1.1 mmol) of 2-phenyl-3-piperazin-1-ylmethyl-quinoline-4-carboxylic acid ((S)-1-cyclohexyl-ethyl)-amide (compound of Description 15), 15 ml of anhydrous THF, 0.23 g (1.6 mmol) of bromoacetamide and 286 ul (1.6 mmol) of diisopropylethyl amine was stirred at room temperature for 16 h. The solvent was concentrated and the residue was dissolved in AcOEt. The organic phase was thoroughly washed with water, dried over MgSO₄ and concentrated affording the title compound as white crystals.

### Example 20

### 3-{4-[4-((S)-1-Cyclohexyl-ethylcarbamoyl)-2-phenyl-quinolin-3-ylmethyl]-piperazin-1-yl}-2-phenyl-propionic acid ethyl ester

Starting from 2-phenyl-3-piperazin-1-ylmethyl-quinoline-4-carboxylic acid ((S)-1-cyclohexyl-ethyl)-amide (compound of Description 15) and following the procedure of description 21) afforded the title compound as a white solid. yield 85.6%

### Example 21

### 3-[4-(2-Methanesulf nyl- thyl)-piperazin-1-ylmethyl]-2-phenyl-quinoline4-carboxylic Acid ((S)-1-cyclohexyl-ethyl)-amide

A mixture of 0.3 g (0.66 mmol) of 2-phenyl-3-piperazin-1-ylmethyl-quinoline-4-carboxylic acid ((S)-1-cyclohexyl-ethyl)-amide (compound of Description 15), 120 ul (1.4 mmol) of methylvinyl sulfone and 7 ml of isopropanol was stirred at reflux for 15 h. The solvent was concentrated and the residue was purified by flash chromatography over 40 g silicagel (eluent: CH₂Cl₂/MeOH:96/4) affording the title compound as white crystals.

### Example 22

### 3-{4-[4-((S)-1-Cyclohexyl-ethylcarbamoyl)-2-phenyl-quinolin-3-ylmethyl]-piperazin-1-yl}-2-phenyl-propionic Acid

Applying the procedure of Example 17 to the ester of Example 20 afforded the title compound as white crystals.

### Example 23

### 3-(4-Cyanomethyl-piperazin-1-ylmethyl)-2-phenyl-quinoline-4-carboxylic Acid ((S)-1-cyclohexyl-ethyl)-amide

Using the procedure of Example 19 but replacing the bromoacetamide by bromoacetonitrile afforded the title compound as a white solid.

### Example 24

### 6-Fluoro-2-phenyl-3-piperazin-1-ylmethylquinoline-4-carboxylic Acid ((S)-1-cyclohexylethyl)-amide

TFA (3 ml) was added dropwise at room temperature to a solution of 4-[4-((S)-1-cyclohexylethylcarbamoyl)-6-fluoro-2-phenylquinolin-3-ylmethyl]-piperazine-1-carboxylic acid tert-butyl ester (0.25 g, 0.4 mmol, compound prepared in Description 26) in CH₂Cl₂ (20 ml). Stirring was continued for additional 3 hours. The solvent was concentrated under vacuum and the residue was basified with 1N NaOH solution and extracted with ethyl acetate. The organic layer was dried over Na₂SO₄, filtered and evaporated to give, after triturating with diisopropyl ether, the title compound (0.15 g).Yield: 79%

### Example 25

### 6-Chl ro-2-phenyl-3-piperazin-1-ylmethylquinoline-4-carboxylic Acid ((S)-1-cyclohexylethyl) amide

The compound was prepared following the procedure of Example 24, according to the Description 23-26 starting from 5-chloroisatin (CAS [17630-76-1]).

### Example 26

### 3-(3-Oxopiperazin-1-ylmethyl)-2-phenylquinoline4-carboxylic Acid ((S)-1-cyclohexylethyl)-amide

A solution of: 3-bromomethyl-2-phenylquinoline-4-carboxylic acid ((S)-1-cyclohexylethyl)-amide (0.3 g, 0.7 mmol; compound prepared from isatine (CAS [91-56-5]) according to Description 23-25), piperazin-2-one (0.1 g, 1 mmol, CAS [5625-67-2]) and ethyldiisopropylamine (0.3 ml, 1.8 mmol) in dry THF (30 ml) was stirred for 24 hours at room temperature. The solvent was evaporated to dryness in vacuo and the residue was re-dissolved in AcOEt. This mixture was washed with a dilute NaOH solution, with water and dried over Na₂SO₄. After evaporating to dryness, the residue was purified by flash chromatography (eluent Ethyl acetate/ Methanol NH₄OH=90/10/0.1) to afford 0.2 g of the desired compound. Yield 60%

### Example 27

### 3-(3-Oxo-4-phenylpiperazin-1-ylmethyl)-2-phenylquinoline-4-carboxylic Acid ((S)-1-cyclohexylethyl)-amide

The compound was prepared following the procedure of Example 26 starting from 1-phenylpiperazin-2-one and 3-bromomethyl-2-phenylquinoline-4-carboxylic acid ((S)-1-cyclohexylethyl)-amide (prepared from isatine (CAS [91-56-5]) according to Description 23-25).

### Example 28

### 3-(4-Methyl-3,5-dioxopiperazin-1-ylmethyl)-2-phenylquinoline4-carboxylic Acid ((S)-1-cyclohexylethyl)-amide

A solution of {carboxymethyl-[4-((S)-1-cyclohexylethylcarbamoyl)-2-phenyl-quinolin-3-ylmethyl]amino}-acetic acid (0.1 g, 0.2 mmol, prepared as in Description 27) in acetamide (4 ml) was stirring for 8 hours at 160° C. then a saturated solution of NaCl was added. The mixture was extracted with EtOAc and the organic layer was dried over Na₂SO₄, filtered and evaporated to give, after purification by flash chromatography (eluent: hexane/ethyl acetate=6/4), 50 mg of the title compound. Yield: 50%

### Example 29

### 3-(3,5-Dioxopiperazin-1-ylmethyl)-2-phenylquinoline-4-carboxylic Acid ((S)-1-cyclohexylethyl)-amide

A solution of {carboxymethyl-[4-((S)- 1-cyclohexylethylcarbamoyl)-2-phenyl-quinolin-3-ylmethyl]amino}-acetic acid (0.1 g, 0.2 mmol, prepared as in Description 27) in 30% solution of NH₃ (15 ml) was evaporated to dryness. The obtained yellow solid compound was heated for 3 hours at 170° C. The crude compound was purified by flash chromatography (eluent: hexane/ethyl acetate=6/4) to give 54 mg of the title compound. Yield: 56%.

### Example 30

### 3-(2-Oxopiperazin-1-ylmethyl)-2-phenylquinoline-4-carboxylic Acid ((S)-1-cyclohexylethyl)-amide

To a solution of 4-[4-((S)-1-Cyclohexylethylcarbamoyl)-2-phenylquinolin-3-ylmethyl]-3-oxopiperazine-1-carboxylic acid tert-butyl ester (0.5 g, 1 mmol, prepared as in Description 28) in CH₂Cl₂ (20 ml), TFA (2 ml) was added drop-wise at room temperature. Stirring was continued overnight. The solvent was evaporated under vacuum and the residue was basified with a saturated solution of K₂CO₃ and extracted with ethyl acetate. The organic layer was dried over Na₂SO₄, filtered and evaporated. The residue was purified on column chromatography (CH₂Cl₂/MeOH/NH₄OH=90:10:1) to give the title compound (0.3 g). Yield 63%

### Example 31

### 3-(2,5-Dioxo-piperazin-1-ylmethyl)-2-phenyl-quinoine4-carboxylic Acid ((S)1-cyclohexylethyl)-amide

NaH (0.06 g, 2.2 mmol) was added portion-wise at room temperature to a suspension of piperazine-2,5-dione (0.5 g, 4 mmol, CAS [106-57-0]) in DMF (10 ml) and DMSO (3 ml). The dark solution was stirred for 30 minutes then a solution of 3-bromomethyl-2-phenylquinoline-4-carboxylic acid ((S)-1-cyclohexyl-ethyl)-amide (1 g, 2.2 mmol, prepared from isatine (CAS [91-56-5]) according to Description 23-25) in DMF (5 ml) was added. The mixture was stirred for additional 4 hours and then was poured in a saturated solution of NaCl. The obtained precipitate was filtered by suction and dried in vacuum oven to yield the title compound ( 0.5 g, 1 mmol). Yield 45%

### Example 32

### 6-Fluoro-3-(3-oxo-piperazin-1-ylmethyl)-2-phenyl-quinoline-4-carboxylic Acid ((S)-1-cyclohexyl-ethyl)-amide

The compound was prepared following the procedure of Example 13 starting from 3-bromomethyl-6-fluoro-2-phenylquinoline-4-carboxylic acid ((S)-1-cyclohexylethyl)-amide (prepared from 5-fluoroisatine (CAS [443-69-6]) according to Description 23-25).

### Example 33

### 3-(4-Benzyl-3-oxopiperazin-1-ylmethyl)-2-phenylquinoline-4-carboxylic Acid ((S)-1-cyclohexylethyl)-amide

NaH ( 0.006 g, 2.2 mmol) was added portion-wise at 0° C. to a suspension of 3-(3-oxo-4-phenylpiperazin- 1-ylmethyl)-2-phenylquinoline-4-carboxylic acid ((S)-1-cyclohexylethyl)-amide (1 g, 2.2 mmol, prepared as in Example 26) in DMF (10 ml). The dark solution was stirred for 10 minutes at 0° C. and then a benzylbromide (0.26 ml, 2.2 mmol) was added dropwise. The mixture was stirred for additional 2 hours at room temperature and then was poured in a saturated solution of NaCl, and extracted with ethyl acetate. The organic layer was dried over Na₂SO₄, filtered and evaporated. The residue was purified on column chromatography (Ethyl Acetate/hexane=4/6) to give the title compound (0.7 g) as a pale yellow solid. Yield 57%

### Example 34

### 7-Chloro-3-(3-oxopiperazin-1-ylmethyl)-2-phenyl-quinoline-4-carboxylic Acid ((S)-1-cyclohexyl-ethyl)-amide

The compound was prepared following the procedure of Example 26 starting from 3-bromomethyl-7-chloro-2-phenylquinoline-4-carboxylic acid ((S)-1-cyclohexylethyl)-amide (prepared from 6-chloroisatin (CAS [6341-92-0]) according to Description 23-25).

### Example 35

### 7-Fluoro-3-(3-oxopiperazin-1-ylmethyl)-2-phenylquinoline-4-carboxylic Acid ((S)-1-cyclohexyl-ethyl)-amide

The compound was prepared following the procedure of Example 26 starting from 3-bromomethyl-7-fluoro-2-phenyl-quinoline-4-carboxylic Acid ((S)-1-cyclohexyl-ethyl)-amide (prepared from 6-fluoroisatine (CAS [324-03-8]) according to Description 23-25).

### Example 36

### 3-(3-Oxo-4-propyl-piperazin-1-ylmethyl)-2-phenyl-quinoline-4-carboxylic Acid ((S)-1-cyclohexyl-ethyl)-amide

NaH ( 0.006 g, 2.2 mmol) was added portion-wise at 0° C. to a suspension of 3-(3-oxo-4-phenylpiperazin-1-ylmethyl)-2-phenylquinoline-4carboxylic acid ((S)- 1-cyclohexylethyl)-amide (1 g, 2.2 mmol, prepared as in Example 26) in DMF (10 ml). The dark solution was stirred for 10 minutes at 0° C. and then propylbromide (0.27 g, 2.2 mmol) was added drop-wise. The mixture was stirred for additional 2 hours at room temperature and the was poured in a saturated solution of NaCl, and extracted with ethyl acetate. The organic layer was dried over Na₂SO₄, filtered and evaporated. The residue was purified on column chromatography (Ethyl Acetate / hexane=4/6) to give the title compound (0.5 g) as a pale yellow solid. Yield 44%

### Example 37

### 3-[4-(2-Hydroxyethyl)-3-oxopiperazin-1-ylmethyl]-2-phenylquinoline-4-carboxylic Acid ((S)-1-cyclohexyl-ethyl)-amide Dihydrochloride

NaH ( 0.024 g, 0.6 mmol) was added portion-wise at 0° C. to a suspension of 3-(3-oxo-4-phenylpiperazin-1-ylmethyl)-2-phenylquinoline-4-carboxylic acid ((S)-1-cyclohexylethyl)-amide (0.24 g, 0.5 mmol, prepared as in Example 26) in DMF (2 ml). The dark solution was stirred for 10 minutes at 0° C. and then a solution of 2-(2-bromo-ethoxy)tetrahydropyran (0.1 g, 0.5 mmol, CAS [17739-45-6]) in THF (2 ml) was added drop-wise. The mixture was stirred for additional 2 hours at room temperature, poured in a saturated solution of NaCl, and extracted with ethyl acetate. The organic layer was dried over Na₂SO₄, filtered and evaporated. The crude residue was re-dissolved in MeOH (4 ml) and a solution of HCl in Et₂O (0.5 ml) was added at 0° C. The mixture was stirred for additional 15 minutes at 0° C. The solvent was evaporated to give 150 mg of the title compound. Yield:58%.

### Example 38

### 8-(3-Oxo-piperazin-1-ylmethyl)-7-phenyl-2,3-dihydro-[1,4]dioxino[2,3-g]quinoline-9-carboxylic Acid ((S)-1-cycloh xyl-ethyl)-amide

A solution of 8-(3-oxopiperazin-1-ylmethyl)-7-phenyl-2,3-dihydro[1,4]dioxino[2,3-g]quinolinecarboxylic acid (100 mg, 0.25 mmol, prepared as in Description 32) TEA (0.14 ml, 1 mmol) and HBTU (95 mg, 0.25 mmol) was stirred for 30 minutes at room temperature. 0.075 ml of (S)-1-cyclohexylethylamine was added at room temperature and the reaction was left to stir overnight. The solvent was evaporated under vacuum and the solid was re-dissolved in ethyl acetate and washed with water, 10% NaHCO₃ and a saturated solution of NaCl; the organic layer was evaporated and the crude product was purified by flash chromatography (CH₂Cl₂/MeOH/NH₄OH=99/1/0.1) to give 80 mg of the title compound. Yield 60%

### Example 39

### 8-Fluoro-3-(3-oxo-piperazin-1-ylmethyl)-2-phenylquinoline-4-carboxylic acid ((S)-1-cyclohexyl-ethyl)-amide

The compound was prepared following the procedure of Example 26 starting from 3-bromomethyl-8-fluoro-2-phenylquinoline-4-carboxylic acid ((S)-1-cyclohexylethyl)-amide (prepared from 7-fluoroisatine (CAS [317-20-4]) according to Description 23-25).

### Example 40

### 3-(3-Oxo-piperazin-1-ylmethyl)-2-phenyl-quinoline-4-carboxylic acid ((S)-1-cyclobexyl-propyl)-amide

The compound was prepared following the procedure of Example 38 starting from (S)-1-phenylpropylamine and 3-(3-oxo-piperazin-1-ylmethyl)-2-phenyl-quinoline-4-carboxylic acid (prepared from 3-Methyl-2-phenyl-quinoline-4-carboxylic acid (CAS [43071-45-0]) according to Description 30-32).

### Example 41

### 3-(3-Oxo-piperazin-1-ylmethyl)-2-thiophen-2-yl-quinoline-4-carboxylic Acid ((S)-1-cyclohexylethyl)-amide

The compound was prepared following the procedure of Example 26 starting from 3-bromomethyl-2-thiophen-2-yl-quinoline-4-carboxylic acid ((S)-1-cyclohexylethyl)-amide (prepared from 1-thiophen-2-yl-propan-1-one (CAS [13679-75-9]) and isatine CAS [91-56-5] according to Description 23-25).

### Example 42

### 3-(3-Oxo-piperazin-1-ylmethyl)-2-phenyl-quinoline4-carboxylic Acid ((S)-2-methyl-1-phenyl-propyl)-amide

The compound was prepared following the procedure of Example 38 starting from (S)-2-methyl-1-phenylpropylamine and 3-(3-oxo-piperazin-1-ylmethyl)-2-phenyl-quinoline-4-carboxylic acid (prepared from 3-methyl-2-phenylquinoline-4-carboxylic acid (CAS [43071-45-0]) and according to Description 30-32).

### Example 43

### 3-[3-Oxo-4-(2-piperidin-1-ylethyl)piperazin-1-ylmethyl]-2-phenylquinoline-4-carboxylic acid ((S)-1-cyclohexylethyl)amide

The compound was prepared following the procedure of Example 33 starting from 1-(2-chloro-ethyl)piperidine (CAS [1932-03-2]) and 3-(3-oxo-4-phenylpiperazin-1-ylmethyl)-2-phenylquinoline4-carboxylic acid ((S)-1-cyclohexylethyl)-amide (prepared as in Example 26).

### Example 44

### 2-(4-Fluorophenyl)-3-(3-oxopiperazin-1-ylmethyl)-quinoline4-carboxylic a cid ((S)-1-cyclohexylethyl)-amide

The compound was prepared following the procedure of Example 26 starting from 1-(4-fluorophenyl)propan-1-one (CAS [456-03-1]) and isatin (CAS [91-56-5]) according to Description 23-25).

### Example 45

### 3-(3-Oxopiperazin-1-ylmethyl)-2-(4-trifluoromethylphenyl)quinoline-4-carboxylic Acid ((S)-1-cyclohexylethyl)-amide

The compound was prepared following the procedure of Example 26 starting from 1-(4-trifluoromethylphenyl)-propan-1-one (CAS [711-33-1]) and isatin (CAS [91-56-5]) according to Description 23-25).

### Example 46

### 2-(2-Fluorophenyl)-3-(3-oxopiperazin-1-ylmethyl)-quinoline4-carboxylic Acid ((S)-1-cyclohexyl-ethyl)-amide

The compound was prepared following the procedure of Example 26 starting 1-(2-fluorophenyl)-propan-1-one (CAS [446-22-0]) and isatin (CAS [91-56-5]) according to Description 23-25).

### Example 47

### 3-(3-Oxo-piperazin-1-ylmethyl)-2-phenyl-6-trifluoromethyl-quinoline-4-carboxylic acid ((S)-1-cyclohexyl-ethyl)-amide

The compound was prepared following the procedure of Example 26 starting from 3-bromomethyl-2-phenyl-6-trifluoromethyl-quinoline-4-carboxylic acid ((S)-1-cyclohexyl-ethyl)-amide (prepared from 5-trifluoroisatine (Tetrahedron Letters, 35, 7303, 1994) according to Description 23-25).

### Example 48

### 3-(3-Oxo-piperazin-1-ylmethyl)-2-phenyl-7-trifluoromethyl-quinoline-4-carboxylic acid ((S)-1-cyclohexyl-ethyl)-amide

The compound was prepared following the procedure of Example 26 starting from 3-bromomethyl-2-phenyl-7-trifluoromethyl-quinoline-4-carboxylic acid ((S)-1-cyclohexyl-ethyl)-amide (prepared from 6-trifluoroisatine (Tetrahedron Letters, 35, 7303, 1994) according to Description 23-25).

TABLE 1

| Ex. | Molecular Structure | Molecular Formula | Melting Point | [a]_{D}²⁰ |
|---|---|---|---|---|
| 1 | 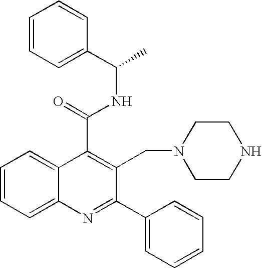 | C₂₉H₃₀N₄O | 198-202° C. | −31.06 (c = 0.5, MeOH) |
| 2 | 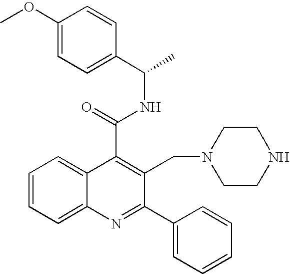 | C₃₀H₃₂N₄O₂ | 109-111° C. | −28.56 (c = 0.5, MeOH) |
| 3 | 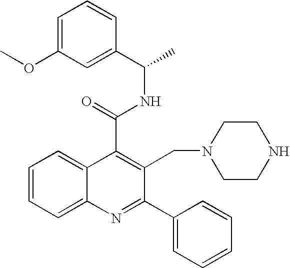 | C₃₀H₃₂N₄O₂ | 147-153° C. | −43.22 (c = 0.5, MeOH) |
| 4 | 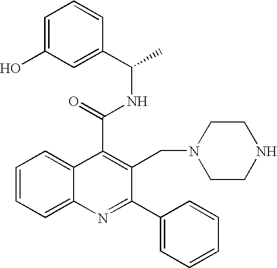 | C₂₉H₃₀N₄O₂ | 240-244° C. | −33.83 (c = 0.5, MeOH) |
| 5 | 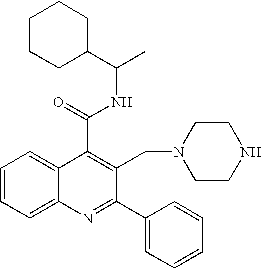 | C₂₉H₃₆N₄O | 118-120° C. | −14.4 (c = 0.5, MeOH) |
| 6 | 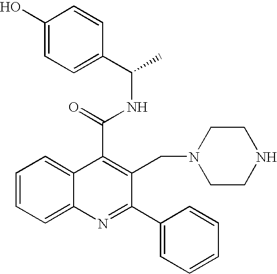 | C₂₉H₃₀N₄O₂ | 163° C. | — |
| 7 | 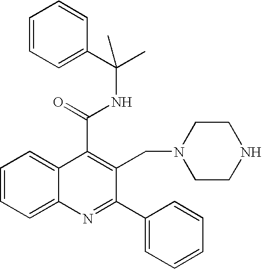 | C₃₀H₃₂N₄O | — | — |
| 8 | 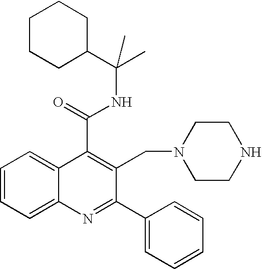 | C₃₀H₃₈N₄O | — | — |
| 9 | 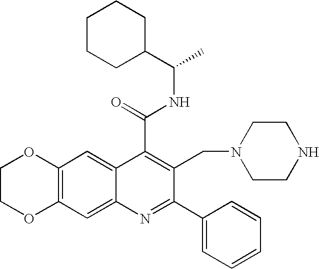 | C₃₁H₃₈N₄O₃ | 135-137° C. | −7.87 (c = 0.15, MeOH) |
| 10 | 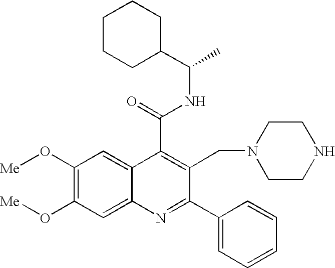 | C₃₁H₄₀N₄O₃ | 130-132° C. | +33.31 (c = 0.25, MeOH) |
| 11 | 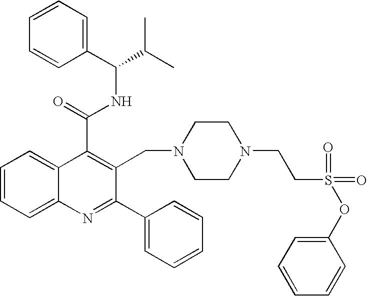 | C₃₉H₄₂N₄O₄S | — | — |
| 12 | 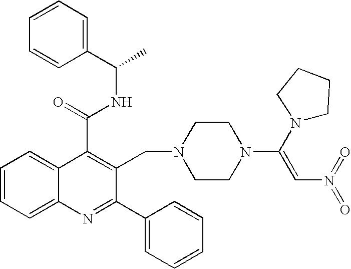 | C₃₅H₃₈N₆O₃ | 164-165° C. | — |
| 13 | 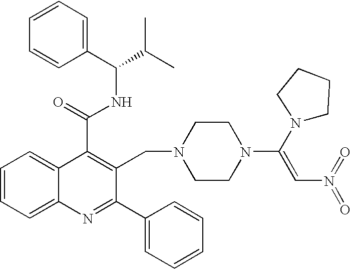 | C₃₇H₄₂N₆O₃ | 170-175° C. | — |
| 14 | 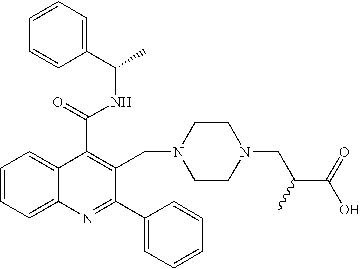 | C₃₃H₃₆N₄O₃ | 137-138° C. | — |
| 15 | 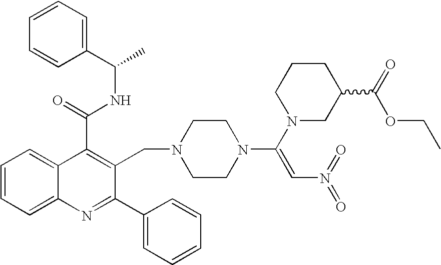 | C₃₉H₄₄N₆O₅ | 120-121° C. | — |
| 16 | 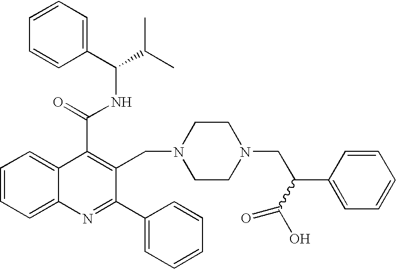 | C₄₀H₄₂N₄O₃ | 156-158° C. | — |
| 17 | 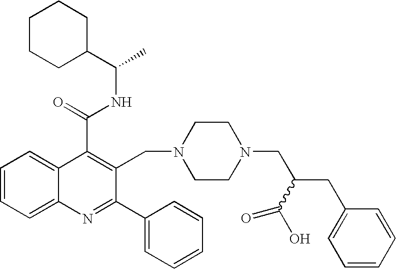 | C₃₉H₄₆N₄O₃ | 136° C. | — |
| 18 | 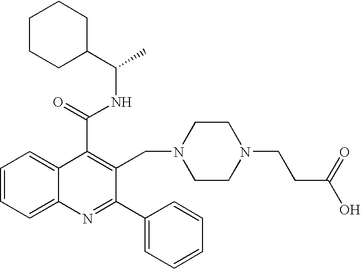 | C₃₂H₄₀N₄O₃ | 192-195° C. | — |
| 19 | 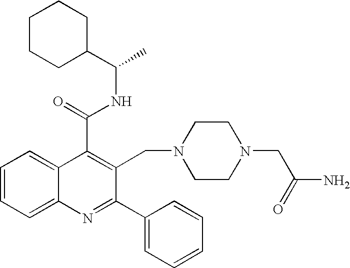 | C₃₁H₃₉N₅O₂ | 125-130° C. | — |
| 20 | 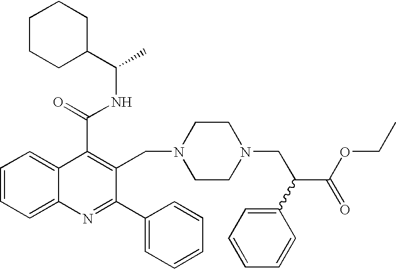 | C₄₀H₄₀N₄O₃S | 90-91° C. | — |
| 21 | 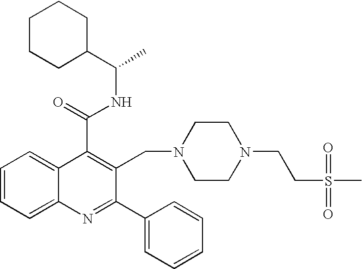 | C₃₂H₄₂N₄O₃S | 120-121° C. | — |
| 22 | 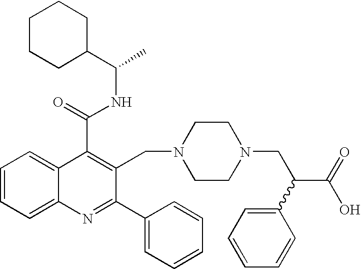 | C₃₈H₄₄N₄O₃ | 144-145° C. | — |
| 23 | 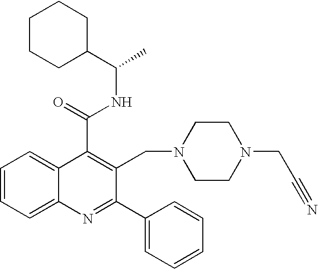 | C₃₁H₃₇N₅O | 96-98° C. | — |
| 24 | 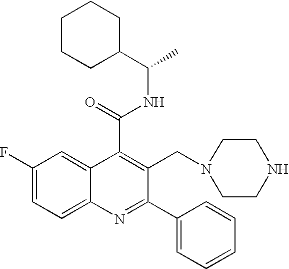 | C₂₉H₃₅FN₄O | 154-158° C. | +7.54 (c = 0.1, MeOH) |
| 25 | 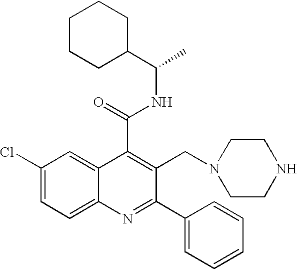 | C₂₉H₃₅ClN₄O | >250° C. | −7.24 (c = 0.5, MeOH) |
| 26 | 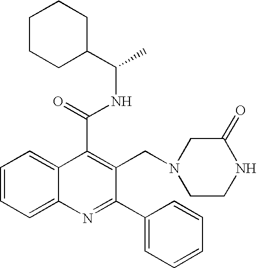 | C₂₉H₃₄N₄O₂ | — | — |
| 27 | 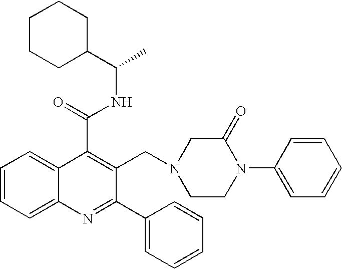 | C₃₅H₃₈N₄O₂ | — | — |
| 28 | 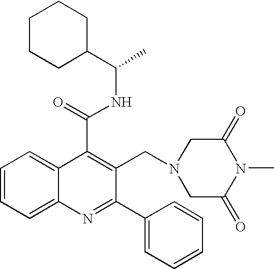 | C₃₀H₃₄N₄O₃ | — | — |
| 29 | 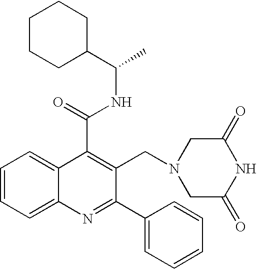 | C₂₉H₃₂N₄O₃ | 124° C. | +5.83 (c = 0.1, MeOH) |
| 30 | 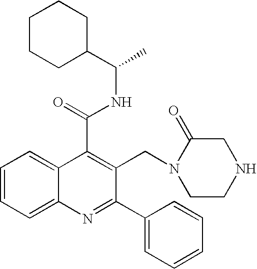 | C₂₉H₃₄N₄O₂ | 212° C. | −5.83 (c = 0.1, MeOH) |
| 31 | 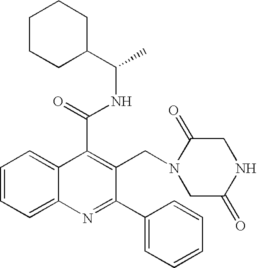 | C₂₉H₃₂N₄O₃ | 251° C. | −7.23 (c = 0.1, MeOH) |
| 32 | 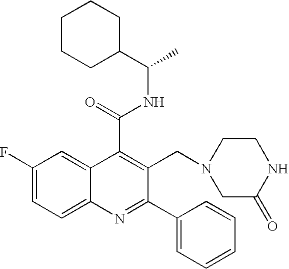 | C₂₉H₃₃FN₄O₂ | 230° C. | +5.8 (c = 0.1, MeOH) |
| 33 | 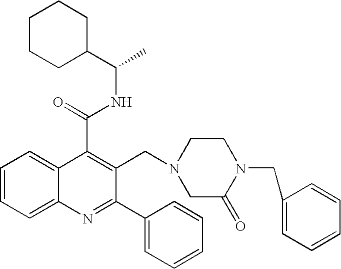 | C₃₆H₄₀N₄O₂ | 181° C. | +8.36 (c = 0.5, MeOH) |
| 34 | 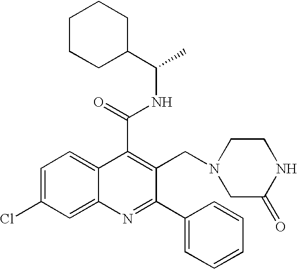 | C₂₉H₃₃ClN₄O₂ | 140-142° C. | — |
| 35 | 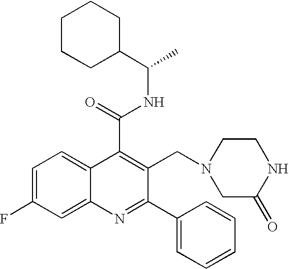 | C₂₉H₃₃FN₄O₂ | 163-165° C. | +12.34 (c = 0.5, MeOH) |
| 36 | 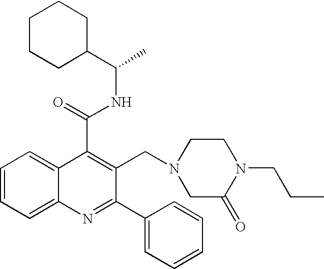 | C₃₂H₄₀N₄O₂ | 150° C. | +11.36 (c = 0.5, MeOH) |
| 37 | 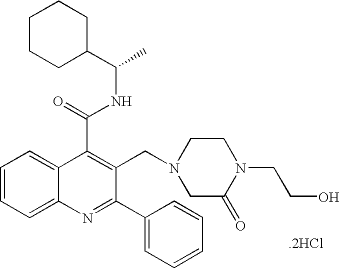 | C₃₁H₃₈N₄O₃.2HCl | 171-173° C. | +16.06 (c = 0.1, MeOH) |
| 38 | 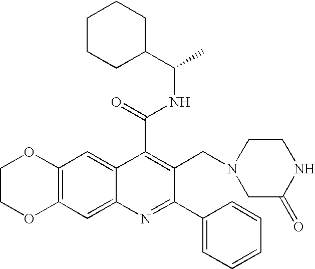 | C₃₁H₃₆N₄O₄ | 144-145° C. | — |
| 39 | 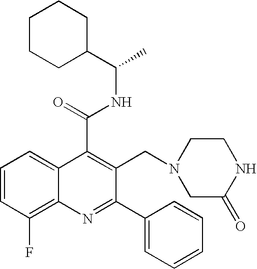 | C₂₉H₃₃FN₄O₂ | 165° C. | +22.67 (c = 0.1, MeOH) |
| 40 | 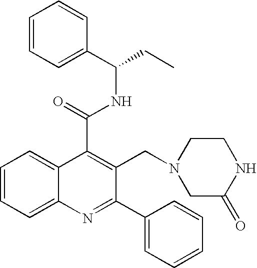 | C₃₀H₃₀N₄O₂ | 160° C. | −47.17 (c = 0.1, MeOH) |
| 41 | 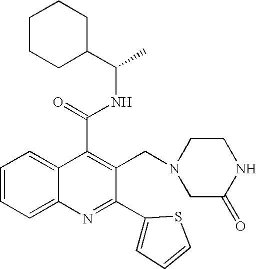 | C₂₇H₃₂N₄O₂S | 225-230° C. | +8.5 (c = 0.2, MeOH) |
| 42 | 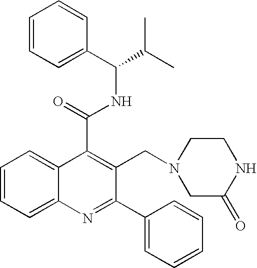 | C₃₁H₃₂N₄O₂ | 137° C. | −47.99 (c = 0.1, MeOH) |
| 43 | 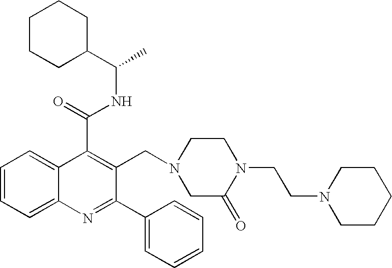 | C₃₆H₄₇N₅O₂ | 160° C. | −4.9 (c = 0.1, MeOH) |
| 44 | 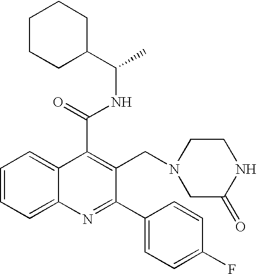 | C₂₉H₃₃FN₄O₂ | — | +10.16 (c = 0.3, EtOH) |
| 45 | 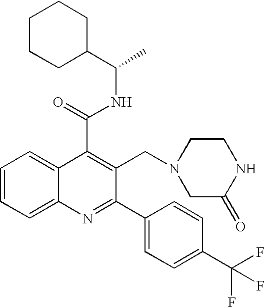 | C₃₀H₃₃F₃N₄O₂ | 214-217° C. | +9.1 (c = 0.2, MeOH) |
| 46 | 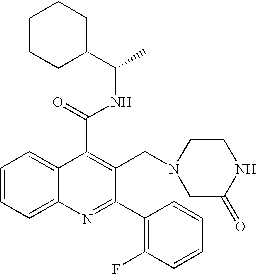 | C₂₉H₃₃FN₄O₂ | 167-169° C. | +14.4 (c = 0.5, MeOH) |
| 47 | 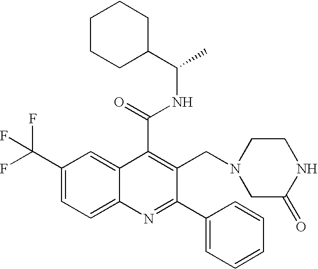 | C₃₀H₃₃F₃N₄O₂ | — | — |
| 48 | 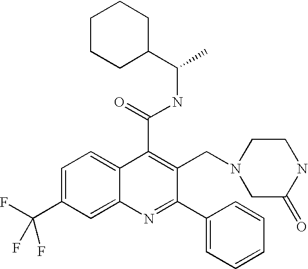 | C₃₀H₃₃F₃N₄O₂ | — | — |

TABLE 2
¹H NMR and/or MS data of compounds of Table 1

| Ex | ¹H NMR (Solvent) ppm and/or MS |
|---|---|
| 1 | ¹H NMR(DMSO-d₆, 343K) δ: 8.90(d br, 1H); 8.01(d, 1H); 7.75(m, 2H); 7.59-7.24(m, 11H); 5.35(m, 1H); 3.54(s, 2H); 2.50(m, |
| | 4H); 2.07(m, 5H); 1.55(d, 3H) |
| | ESI POS; AQA; solvent: MeOH/spray 3kV/skimmer: 20V/probe 135° C.: 451(MH+); 226(MHH++) |
| 2 | ¹H NMR(DMSO-d₆, 343K) δ: 8.81(d br, 1H); 8.00(d, 1H); 7.74(m, 2H); 7.56(m, 3H); 7.49-7.36(m, 5H); 6.93(d, 2H); 5.30(m, |
| | 1H); 3.78(s, 3H); 3.48(s, 2H); 2.41(m, 4H); 1.98(m, 4H); 1.53(d, 3H) |
| | ESI POS; AQA; solvent: MeOH/spray 3kV/skimmer: 20V/probe 135° C.: 481(MH+) |
| 3 | ¹H NMR(DMSO-d₆, 43K; determined as trifluoroacetic salt) δ: 8.92(d br, 1H); 8.42(s br, 2H); 8.03(d, 1H); 7.78(m, 2H); 7.61-7.44 |
| | m, 6H); 7.31(dd, 1H); 7.06(m, 2H); 6.87(dd, 1H); 5.32(m, 1H); 3.79(s, 3H); 3.63(s br, 2H); 2.74(m, 4H); 2.27(m, 4H); 1.53 |
| | (d, 3H) |
| | ESI POS; AQA; solvent: MeOH/spray 3kV/skimmer: 20V/probe 135° C.: 481(MH+) |
| 4 | ¹H NMR(DMSO-d₆, 343K) δ: 8.81(d br, 1H); 8.00(d, 1H); 7.74(m, 2H); 7.56(m, 3H); 7.49-7.36(m, 3H); 7.15(dd, 1H); 6.91(d, |
| | 1H); 6.90(s, 1H); 6.68(d, 1H); 6.63(s vbr, 1H); 5.26(m, 1H); 3.52(s, 2H); 2.44(m, 4H); 2.01(m, 4H); 1.52(d, 3H) |
| | EI; TSQ 700; source 180° C.; 70V; 200 uA: 466(M+.); 465; 424; 394; 380; 329; 300; 273; 261; 217. |
| 5 | ESI POS; AQA; solvent: MeOH/spray 3kV/skimmer: 20V/probe 135° C.: 457(MH+) |
| 6 | ESI POS; AQA; solvent: MeOH/spray 3kV/skimmer: 20V/probe 135° C.: 467(MH+) |
| 7 | ¹H NMR(DMSO-d₆, 343K) δ: 8.67(s br, 1H); 8.00(d, 1H); 7.88(d, 1H); 7.75(dd, 1H); 7.62-7.54(m, 5H); 7.51-7.35(m, 5H); 7.27 |
| | (dd, 1H); 3.59(s, 2H); 2.41(m, 4H); 2.03(m, 4H); 1.81(s, 6H) |
| | ESI POS; AQA; solvent: MeOH/spray 3kV/skimmer: 20V/probe 135° C.: 465(MH+) |
| 8 | ESI POS; AQA; solvent: MeOH/spray 3kV/skimmer: 20V/probe 135° C.: 471(MH+) |
| 9 | ESI POS; AQA; solvent: MeOH/spray 3kV/skimmer: 20V/probe 135° C.: 515(MH+) |
| 10 | ESI POS; AQA; solvent: MeOH/spray 3kV/skimmer: 20V/probe 135° C.: 517(MH+) |
| 11 | ¹H NMR(CDCl₃) δ: 0.95(d, 3H); 1.17(d, 3H); 1.70-2.43(m, 9H); 2.81(t, 2H); 3.29(t, 2H); 3.57(s, 2H); 5.17(m, 1H); 7.16-7.50 |
| | (m, 16H ar); 7.73(td, 1H ar); 7.85-8.05(m, 2H); 8.13(dd, 1H ar) |
| 12 | ¹H NMR(CDCl₃) δ:1.48-2.25(m, 8H); 1.72(d, 3H); 2.85(m, 4H); 3.23(m, 4H); 3.66(s, 2H); 5.53(m, 1H); 6.22(s, 1H); 7.28-7.65 |
| | (12H ar); 7.74(t, 1H ar); 7.99(d br, 1H); 8.12(d, 1H ar) |
| 13 | ¹H NMR(CDCl₃) δ: 0.92(d, 3H); 1.17(d, 3H); 1.60-2.10(m, 8H); 2.22(m, 1H); 2.89(m, 4H); 3.24(m, 4H); 3.53(m, 2H); 5.12(m, |
| | 1H); 6.21(s, 1H); 7.00-7.64(12H); 7.72(t, 1H ar); 7.90(br, 1H ar); 8.12(d, 1H ar) |
| 14 | ¹H NMR(CDCl₃) δ: 1.10(d, 3H); 1.72(d, 3H); 1.88-2.49(m, 11H); 2.70(br, 1H); 3.64(s, 2H); 5.53(m, 1H); 7.25-7.52(m, 8H ar); |
| | 7.53-7.81(m, 3H); 8.01(d, 1H ar); 8.14(d, 1H ar) |
| 15 | ¹H NMR(CDCl₃) δ: 1.23(t, 3H); 1.45-2.25(m, 8H); 1.72(d, 3H); 2.41-3.32(m, 8H); 3.47(m, 1H); 3.66(s, 2H); 4.11(q, 2H); 5.53 |
| | (m, 1H); 6.10(s, 1H); 7.25-7.55(m, 11H); 7.59(t, 1H ar); 7.77(td, 1H ar); 7.99(d, 1H ar); 8.13(dd, 1H ar) |
| 16 | ¹H NMR(CDCl₃) δ: 0.90(d, 3H); 1.14(d, 3H); 1.82-2.70(m, 11H); 3.00(td, 1H); 3.52(s, 2H); 5.09(t, 1H); 6.93(br, 1H); 7.03-7.63 |
| | (m, 17H ar); 7.73(t, 1H ar); 7.91(br, 1H); 8.13(d, 1H ar) |
| 17 | ¹H NMR(DMSO-d₆) δ: 0.95-1.32(m, 8H); 1.45(m, 1H); 1.55-1.88(m, 5H); 2.00-2.35(m, 8H); 2.92(m, 1H); 2.60-2.85(m, 4H); |
| | 3.41(br, 1H); 3.52(s, 2H); 4.00(m, 1H); 7.18(m, 5H ar); 7.48-7.91(m, 8H ar); 8.02(d, 1H); 8.55(d, 1H) |
| 18 | ¹H NMR(CDCl₃) δ: 0.90-1.35(m, 5H); 1.15(d, 3H); 1.45(m, 1H); 1.58-1.90(m, 5H); 1.92-2.30(m, 10H); 2.41(t, 2H); 3.41(br, |
| | 1H); 3.53(s, 2H); 4.02(m, 1H); 7.35-7.90(m, 8H ar); 8.03(d, 1H ar); 8.57(d br, 1H) |
| 19 | ¹H NMR(CDCl₃) δ: 1.00-2.00(11H); 1.29(d, 3H); 2.26(m, 4H); 2.39(m, 4H); 2.93(s, 2H); 3.75(s, 2H); 4.25(m, 1H); 5.45(br, |
| | 1H); 6.90(br, 1H); 7.38-7.69(m, 7H); 7.74(t, 1H ar); 8.04(d, 1H); 8.13(d, 1H ar) |
| 20 | ¹H NMR(CDCl₃) δ: 0.95-1.55(m, 6H); 1.16(t, 3H); 1.27(d, 3H); 1.60-1.98(5H); 2.02-2.55(m, 8H); 3.09(t, 1H); 3.67(m, 1H); |
| | 3.73(s, 2H); 4.00-4.36(4H); 7.27(m, 5H ar); 7.47(m, 5H ar); 7.58(t, 1H ar); 7.73(t, 1H ar); 8.05-8.19(m, 2H ar); 8.28(br, 1H) |
| 21 | ¹H NMR(CDCl₃) δ: 1.00-1.35(m, 5H); 1.28(d, 3H); 1.45(m, 1H); 1.65-1.97(m, 5H); 2.10-2.48(m, 8H); 2.75(t, 2H); 2.94(s, 3H); |
| | 3.04(t, 2H); 3.73(s, 2H); 4.26(m, 1H); 7.47(m, 5H ar); 7.55(br, 1H); 7.59(t, 1H ar); 7.74(t, 1H ar); 8.05(d, 1H ar); 8.13(d, 1H |
| | ar) |
| 22 | ¹H NMR(CDCl₃) δ: 0.93-1.37(m, 5H); 1.27(d, 3H); 1.45(m, 1H); 1.59-1.93(m, 5H); 2.18-2.94(9H); 2.98(t, 1H); 3.66(dd, 1H); |
| | 3.74(s, 2H); 4.25(m, 1H); 6.74(br, 1H); 7.18-7.40(m, 6H); 7.47(m, 5H ar); 7.60(td, 1H ar); 7.75(td, 1H ar); 8.00(dd, 1H ar); 8.15 |
| | (dd, 1H ar) |
| 23 | ¹H NMR(CDCl₃) δ: 0.95-1.99(m, 11H), 1.29(d, 3H); 2.22-2.53(m, 8H); 3.42(s, 2H); 3.76(dd, 2H); 4.28(m, 1H); 7.48(m, 5H ar); |
| | 7.59(td and br, 2H); 7.76(td, 1H ar); 8.08(dd, 1H ar); 8.15(dd, 1H) |
| 24 | ¹H NMR(DMSO-d₆, 303K) δ: 8.55(d br, 1H); 8.10(dd, 1H); 7.70(dt, 1H); 7.58-7.52(m, 2H); 7.51-7.41(m, 4H); 4.01(m, 1H); |
| | 3.52(s, 2H); 2.43(m, 4H); 2.02(m, 4H); 1.85-1.58(m, 4H); 1.48(m, 1H); 1.30-1.01(m, 7H); 1.16(d, 3H) |
| | EI; TSQ 700; source 180 C; 70V; 200 uA: 474(M+); 432; 418; 390; 388; 347; 320; 291; 279; 235; 140; 85 |
| 25 | ¹H NMR(DMSO-d₆, 343K) δ: 8.33(d br, 1H); 8.04(d, 1H); 7.86(d, 1H); 7.76(dd, 1H); 7.57(m, 2H); 7.51-7.41(m, 3H); 4.05(m, |
| | 1H); 3.56(s, 2H); 2.45(m, 4H); 2.04(m, 4H); 1.87-1.61(m, 5H); 1.54(m, 1H); 1.34-1.06(m, 6H); 1.20(d, 3H) |
| | EI; TSQ 700; source 180 C; 70V; 200 uA: 490(M+); 406; 336; 295; 280; 140; 85 |
| 26 | ¹H NMR(DMSO-d₆, 343K) δ: 8.53(s br, 1H); 8.03(d, 1H); 7.85(d, 1H); 7.79(dd, 1H); 7.66(dd, 1H); 7.57-7.42(m, 6H); 3.99(m, |
| | 1H); 3.65(s, 2H); 2.85(m, 2H); 2.65(s, 2H); 2.27(m, 2H); 1.86-1.58(m, 5H); 1.46(m, 1H); 1.29-0.99(m, 5H); 1.16(d, 3H) |
| | ESI POS; AQA; solvent: MeOH/spray 3kV/skimmer: 20V/probe 135° C.: 471(MH+) |
| 27 | ESI POS; AQA; solvent: MeOH/spray 3kV/skimmer: 20V/probe 135° C.: 547(MH+) |
| 28 | ¹H NMR(DMSO-d₆, 343K) δ: 8.34(d br, 1H); 8.03(d, 1H); 7.87(d, 1H); 7.79(dd, 1H); 7.65(dd, 1H); 7.46(m, 5H); 3.99(m, 1H); |
| | 3.78(s, 2H); 3.15(s, 4H); 2.88(s, 3H); 1.84-1.59(m, 5H); 1.47(m, 1H); 1.33-1.05(m, 5H); 1.17(d, 3H) |
| | EI; TSQ 700; source 180 C; 70V; 200 uA: 498(M+); 372; 263; 246; 217 |
| 29 | ¹H NMR(DMSO-d₆, 343K) δ: 10.61(s br, 1H); 8.34(d br, 1H); 8.04(d, 1H); 7.87(d, 1H); 7.79(dd, 1H); 7.65(dd, 1H); 7.50-7.43 |
| | (m, 5H); 4.00(m, 1H); 3.78(s, 2H); 3.01(s, 4H); 1.85-1.59(m, 5H); 1.49(m, 1H); 1.32-1.03(m, 5H); 1.18(d, 3H) |
| | EI; TSQ 700; source 180 C; 70V; 200 uA: 484(M+); 372; 357; 263; 246; 217 |
| 30 | ¹H NMR(DMSO-d₆, 343K) δ: 8.39(d br, 1H); 8.03(d, 1H); 7.85(d, 1H); 7.79(dd, 1H); 7.65(dd, 1H); 7.50-7.39(m, 5H); 4.73(s |
| | br, 2H); 4.02(m, 1H); 2.89(s, 2H); 2.73(m, 2H); 2.58(t, 2H); 2.07(s br, 1H); 1.85-1.59(m, 5H); 1.50(m, 1H); 1.32-1.03(m, 5H); |
| | 1.20(d, 3H) |
| | EI; TSQ 700; source 180 C; 70V; 200 uA: 470(M+); 388; 370; 316; 287; 261 |
| 31 | ¹H NMR(DMSO-d₆, 343K) δ: 8.47(d br, 1H); 8.04(d, 1H); 7.86(d, 1H); 7.81(dd, 1H); 7.67(dd, 1H); 7.59(s br, 1H); 7.44(m, |
| | 5H); 4.78-4.60(m, 2H); 4.01(m, 1H); 3.42(s br, 2H); 3.38(m, 2H); 1.84-1.60(m, 5H); 1.51(m, 1H); 1.34-1.03(m, 5H); 1.20(d, |
| | 3H) |
| | EI; TSQ 700; source 180 C; 70V; 200 uA: 484(M+); 401; 370; 330; 300; 273; 217 |
| 32 | ¹H NMR(DMSO-d₆, 343K) δ: 8.32(d br, 1H); 8.10(dd, 1H); 7.67(dt, 1H); 7.55-7.43(m, 6H); 7.22(s br, 1H); 4.02(m, 1H); 3.70 |
| | (s, 2H); 2.90(m, 2H); 2.69(s, 2H); 2.31(m, 2H); 1.86-1.61(m, 5H); 1.51(m, 1H); 1.32-1.05(m, 5H); 1.19(d, 3H) |
| | EI; TSQ 700; source 180 C; 70V; 200 uA: 488(M+); 390; 262; 279; 264; 235 |
| 33 | ¹H NMR(DMSO-d₆, 343K) δ: 8.53(s br, 1H); 8.03(d, 1H); 7.83(d, 1H); 7.79(dd, 1H); 7.66(d, 1H); 7.50(m, 2H); 7.49(m, 3H); |
| | 7.34(dd, 2H); 7.26(dd, 1H); 7.11(d, 2H); 4.40(s, 2H); 3.99(m, 1H); 3.67(s, 2H); 2.89(m, 2H); 2.82(s, 2H); 2.36(m, 2H); |
| | 1.83-1.58(m, 5H); 1.44(m, 1H); 1.28-0.99(m, 8H) |
| | EI; TSQ 700; source 180 C; 70V; 200 uA: 560(M+.); 469; 372; 263; 217 |
| 34 | ¹H NMR(DMSO-d₆, 343K) δ: 8.54(s br, 1H); 8.09(d, 1H); 7.85(d, 1H); 7.71(dd, 1H); 7.56-7.43(m, 6H); 3.98(m, 1H); 3.64(s, |
| | 2H); 2.85(m, 2H); 2.65(s, 2H); 2.26(m, 2H); 1.83-1.58(m, 5H); 1.45(m, 1H); 1.28-0.98(m, 5H); 1.15(d, 3H) |
| | EI; TSQ 700; source 180 C; 70V; 200 uA: 504(M+); 406; 297; 280; 253 |
| 35 | ¹H NMR(DMSO-d₆, 343K) δ: 8.55(s br, 1H); 7.89(dd, 1H); 7.78(dd, 1H); 7.62(dt, 1H); 7.55-7.44(m, 6H); 3.99(m, 1H); 3.64(s, |
| | 2H); 2.85(m, 2H); 2.65(s, 2H); 2.26(m, 2H); 1.83-1.58(m, 5H); 1.44(m, 1H); 1.28-0.98(m, 5H); 1.15(d, 3H) |
| | EI; TSQ 700; source 180 C; 70V; 200 uA: 488(M+); 390; 334; 281; 264; 235 |
| 36 | ¹H NMR(DMSO-d₆, 343K) δ: 8.36(d br, 1H); 8.02(d, 1H); 7.86(d, 1H); 7.78(dd, 1H); 7.64(dd, 1H); 7.55-7.43(m, 5H); 4.02(m, |
| | 1H); 3.67(s, 2H); 3.14(dd, 2H); 2.97(m, 2H); 2.74(s, 2H); 2.38(m, 2H); 1.86-1.60(m, 5H); 1.49(m, 1H); 1.41(m, 2H); 1.29-1.26 |
| | (m, 5H); 1.18(d, 3H); 0.78(t, 3H) |
| | EI; TSQ 700; source 180 C; 70V; 200 uA: 512(M+); 372; 263; 246; 217; 141 |
| 37 | ¹H NMR(DMSO-d₆, 343K) δ: 8.52(d br, 1H); 8.03(d, 1H); 7.85(d, 1H); 7.79(dd, 1H); 7.66(dd, 1H); 7.54-7.42(m, 5H); 4.58(t, |
| | 1H); 3.99(m, 1H); 3.63(s, 2H); 3.39(m, 2H); 3.21(m, 2H); 3.06(m, 2H); 2.72(s, 2H); 2.35(m, 2H); 1.84-1.58(m, 5H); 1.45(m, |
| | 1H); 1.28-0.99(m, 8H) |
| | EI; TSQ 700; source 180 C; 70V; 200 uA: 514(M+); 372; 261; 246; 217 |
| 38 | ¹H NMR(DMSO-d₆, 343K) δ: 8.46(d br, 1H); 7.52-7.40(m, 6H); 7.39(s, 1H); 7.17(s, 1H); 4.39(s, 4H); 3.96(m, 1H); 3.58(s, |
| | 2H); 2.86(m, 2H); 2.64(s, 2H); 2.24(m, 2H); 1.83-1.57(m, 5H); 1.45(m, 1H); 1.30-0.99(m, 5H); 1.13(d, 3H) |
| | EI; TSQ 700; source 180 C; 70V; 200 uA: 528(M+); 430; 345; 319; 304; 277 |
| 39 | ¹H NMR(DM50-d₆, 343K) δ: 8.31(d br, 1H); 7.70-7.45(m, 8H); 7.21(s br, 1H); 4.02(m, 1H); 3.70(s, 2H); 2.89(m, 2H); 2.69(s, |
| | 2H); 2.31(m, 2H); 1.86-1.61(m, 5H); 1.50(m, 1H); 1.33-1.07(m, 5H); 1.19(d, 3H) |
| | EI; TSQ 700; source 180 C; 70V; 200 uA: 488(M+); 390; 291; 281; 264; 235 |
| 40 | ¹H NMR(DMSO-d₆, 343K) δ: 8.92(d br, 1H); 8.02(d, 1H); 7.75(dd, 1H); 7.71(d br, 1H); 7.59-7.41(m, 8H); 7.38(dd, 2H); 7.28 |
| | (dd, 1H); 7.15(s br, 1H); 5.08(dt, 1H); 3.58(s, 2H); 2.81(m, 2H); 2.54(s, 2H); 2.17(m, 2H); 1.88(m, 2H); 0.96(t, 3H) |
| | EI; TSQ 700; source 180 C; 70V; 200 uA: 478(M+); 433; 380; 351; 261; 246; 217 |
| 41 | ¹H NMR(DMSO-d₆, 343K) δ: 8.59(d br, 1H); 8.01(d, 1H); 7.92(m, 1H); 7.82-7.61(m, 5H); 7.21(dd, 1H); 4.08-3.70(m, 3H); |
| | 3.11-2.76(m, 4H); 2.53(s, 2H); 1.83-1.59(m, 5H); 1.47(m, 1H); 1.28-1.01(m, 5H); 1.17(d, 3H) |
| | EI; TSQ 700; source 180 C; 70V; 200 uA: 476(M+); 378; 322; 288; 252 |
| 42 | ¹H NMR(DMSO, 343K) δ: 8.94(d br, 1H); 8.01(d, 1H); 7.75(dd, 1H); 7.64(m, 1H); 7.57-7.25(m, 11H); 7.12(s br, 1H); 4.90(t, |
| | 1H); 3.51(s br, 2H); 2.77(m, 2H); 2.50(s, 2H); 2.10(m, 3H); 1.09(d, 3H); 0.84(d, 3H) |
| | EI; TSQ 700; source 180 C; 70V; 200 uA: 492(M+); 351; 261; 246; 217; 133 |
| 43 | ¹H NMR(DMSO-d₆, 343K) δ: 8.29(d br, 1H); 8.02(d, 1H); 7.88(d, 1H); 7.77(dd, 1H); 7.64(dd, 1H); 7.54(m, 2H); 7.46(m, 3H); |
| | 4.03(m, 1H); 3.66(s, 2H); 3.27(t, 2H); 3.06(m, 2H); 2.74(s, 2H); 2.38(m. 2H); 2.32(m, 6H); 1.87-1.60(m, 5H); 1.54-1.33(m, |
| | 7H); 1.29-1.04(m, 5H); 1.19(d, 3H). |
| | EI; TSQ 700; source 180 C; 70V; 200 uA: 581(M+); 427; 210; 111; 98 |
| 44 | ¹H NMR(DMSO-d₆, 343K) δ: 8.31(d br, 1H); 8.03(d, 1H); 7.87(d, 1H); 7.78(dd, 1H); 7.64(dd, 1H); 7.61(dd, 2H); 7.27(dd, |
| | 2H); 7.26(s br, 1H); 4.03(m, 1H); 3.68(s, 2H); 2.91(m, 2H); 2.71(s, 2H); 2.34(m, 2H); 1.86-1.61(m, 5H); 1.51(m, 1H); 1.32-1.05 |
| | (m, 5H); 1.20(d, 3H) |
| | EI; TSQ 700; source 180 C; 70V; 200 uA: 488(M+.); 390; 281; 264; 235 |
| 45 | ¹H NMR(DMSO-d₆, 343K) δ: 8.33(d br, 1H); 8.05(d, 1H); 7.89(d, 1H); 7.79(m, 5H); 7.67(dd, 1H); 7.21(s br, 1H); 4.05(m, |
| | 1H); 3.70(s, 2H); 2.85(m, 2H); 2.68(s, 2H); 2.32(m, 2H); 1.88-1.61(m, 5H); 1.51(m, 1H); 1.34-1.05(m, 5H); 1.21(d, 3H) |
| | EI; TSQ 700; source 180 C; 70V; 200 uA: 538(M+); 440; 412; 366; 331; 314; 285 |
| 46 | ¹H NMR(DMSO-d₆, 343K) δ: 8.39(d br, 1H); 8.04(d, 1H); 7.88(d, 1H); 7.79(dd, 1H); 7.67(dd, 1H); 7.54-7.40(m, 2H); 7.34-7.21 |
| | (m, 2H); 7.20(s br, 1H); 4.04(m, 1H); 3.58(s, 2H); 2.88(m, 2H); 2.64(s, 2H); 2.29(m, 2H); 1.89-1.60(m, 5H); 1.51(m, 1H); |
| | 1.38-1.06(m, 5H); 1.20(d, 3H) |
| | EI; TSQ 700; source 180 C; 70V; 200 uA: 488(M+); 384 |

TABLE 3
Chemical names of parent compounds of Table 1(names generated by Beilstein's Autonom)

| Ex | Chemical name |
|---|---|
| 1 | 2-Phenyl-3-piperazin-1-ylmethyl-quinoline-4-carboxylic acid((S)-1-phenyl-ethyl)-amide |
| 2 | 2-Phenyl-3-piperazin-1-ylmethyl-quinoline-4-carboxylic acid [(S)-1-(4-methoxy-phenyl)-ethyl]-amide |
| 3 | 2-Phenyl-3-piperazin-1-ylmethyl-quinoline-4-carboxylic acid [(S)-1-(3-methoxy-phenyl)-ethyl]-amide |
| 4 | 2-Phenyl-3-piperazin-1-ylmethyl-quinoline-4-carboxylic acid [(S)-1-(3-hydroxy-phenyl)-ethyl]-amide |
| 5 | 2-Phenyl-3-piperazin-1-ylmethyl-quinoline-4-carboxylic acid((R)-1-cyclohexyl-ethyl)-amide |
| 6 | 2-Phenyl-3-piperazin-1-ylmethyl-quinoline-4-carboxylic acid [(S)-1-(4-hydroxy-phenyl)-ethyl]-amide |
| 7 | 2-Phenyl-3-piperazin-1-ylmethyl-quinoline-4-carboxylic acid(1-methyl-1-phenyl-ethyl)-amide |
| 8 | 2-Phenyl-3-piperazin-1-ylmethyl-quinoline-4-carboxylic acid(1-cyclohexyl-1-methyl-ethyl)-amide |
| 9 | 7-Phenyl-8-piperazin-1-ylmethyl-2,3-dihydro-[1,4]dioxino[2,3-g]quinoline-9-carboxylic acid((S)-1-cyclohexyl-ethyl)-amide |
| 10 | 6,7-Dimethoxy-2-phenyl-3-piperazin-1-ylmethyl-quinoline-4-carboxylic acid((S)-1-cyclohexyl-ethyl)-amide |
| 11 | 2-{4-[4-((S)-2-Methyl-1-phenyl-propylcarbamoyl)-2-phenyl-quinolin-3-ylmethyl]-piperazin-1-yl}-ethanesulfonic acid phenyl ester |
| 12 | 3-[4-(2-Nitro-1-pyrrolidin-1-yl-vinyl)-piperazin-1-ylmethyl]-2-phenyl-quinoline-4-carboxylic acid((S)-1-phenyl-ethyl)-amide |
| 13 | 3-[4-(2-Nitro-1-pyrrolidin-1-yl-vinyl)-piperazin-1-ylmethyl]-2-phenyl-quinoline-4-carboxylic acid((S)-2-methyl-1-phenyl-propyl)- |
| | amide |
| 14 | 2-Methyl-3-{4-[2-phenyl-4-((S)-1-phenyl-ethylcarbamoyl)-quinolin-3-ylmethyl]-piperazin-1-yl}-propionic acid |
| 15 | 1-(2-Nitro-1-{4-[2-phenyl-4-((S)-1-phenyl-ethylcarbamoyl)-quinolin-3-ylmethyl]-piperazin-1-yl}-vinyl)-piperidine-3-carboxylic |
| | acid ethyl ester |
| 16 | 3-{4-[4-((S)-2-Methyl-1-phenyl-propylcarbamoyl)-2-phenyl-quinolin-3-ylmethyl]-piperazin-1-yl}-2-phenyl-propionic acid |
| 17 | 2-Benzyl-3-{4-[4-((S)-1-cyclohexyl-ethylcarbamoyl)-2-phenyl-quinolin-3-ylmethyl]-piperazin-1-yl}-propionic acid |
| 18 | 3-{4-[4-((S)-1-Cyclohexyl-ethylcarbamoyl)-2-phenyl-quinolin-3-ylmethyl]-piperazin-1-yl}-propionic acid |
| 19 | 3-(4-Carbamoylmethyl-piperazin-1-ylmethyl)-2-phenyl-quinoline-4-carboxylic acid((S)-1-cyclohexyl-ethyl)-amide |
| 20 | 3-{4-[4-((S)-1-Cyclohexyl-ethylcarbamoyl)-2-phenyl-quinolin-3-ylmethyl]-piperazin-1-yl}-2-phenyl-propionic acid ethyl ester |
| 21 | 3-[4-(2-Methanesulfonyl-ethyl)-piperazin-1-ylmethyl]-2-phenyl-quinoline-4-carboxylic acid((S)-1-cyclohexyl-ethyl)-amide |
| 22 | 3-{4-[4-((S)-1-Cyclohexyl-ethylcarbamoyl)-2-phenyl-quinolin-3-ylmethyl]-piperazin-1-yl}-2-phenyl-propionic acid |
| 23 | 3-(4-Cyanomethyl-piperazin-1-ylmethyl)-2-phenyl-quinoline-4-carboxylic acid((S)-1-cyclohexyl-ethyl)-amide |
| 24 | 6-Fluoro-2-phenyl-3-piperazin-1-ylmethyl-quinoline-4-carboxylic acid((S)-1-cyclohexyl-ethyl)-amide |
| 25 | 6-Chloro-2-phenyl-3-piperazin-1-ylmethyl-quinoline-4-carboxylic acid((S)-1-cyclohexyl-ethyl)-amide |
| 26 | 3-(3-Oxo-piperazin-1-ylmethyl)-2-phenyl-quinoline-4-carboxylic acid((S)-1-cyclohexyl-ethyl)-amide |
| 27 | 3-(3-Oxo-4-phenyl-piperazin-1-ylmethyl)-2-phenyl-quinoline-4-carboxylic acid((S)-1-cyclohexyl-ethyl)-amide |
| 28 | 3-(4-Methyl-3,5-dioxo-piperazin-1-ylmethyl)-2-phenyl-quinoline-4-carboxylic acid((S)-1-cyclohexyl-ethyl)-amide |
| 29 | 3-(3,5-Dioxo-piperazin-1-ylmethyl)-2-phenyl-quinoline-4-carboxylic acid((S)-1-cyclohexyl-ethyl)-amide |
| 30 | 3-(2-Oxo-piperazin-1-ylmethyl)-2-phenyl-quinoline-4-carboxylic acid((S)-1-cyclohexyl-ethyl)-amide |
| 31 | 3-(2,5-Dioxo-piperazin-1-ylmethyl)-2-phenyl-quinoline-4-carboxylic acid((S)-1-cyclohexyl-ethyl)-amide |
| 32 | 6-Fluoro-3-(3-oxo-piperazin-1-ylmethyl)-2-phenyl-quinoline-4-carboxylic acid((S)-1-cyclohexyl-ethyl)-amide |
| 33 | 3-(4-Benzyl-3-oxo-piperazin-1-ylmethyl)-2-phenyl-quinoline-4-carboxylic acid((S)-1-cyclohexyl-ethyl)-amide |
| 34 | 7-Chloro-3-(3-oxo-piperazin-1-ylmethyl)-2-phenyl-quinoline-4-carboxylic acid((S)-1-cyclohexyl-ethyl)-amide |
| 35 | 7-Fluoro-3-(3-oxo-piperazin-1-ylmethyl)-2-phenyl-quinoline-4-carboxylic acid((S)-1-cyclohexyl-ethyl)-amide |
| 36 | 3-(3-Oxo-4-propyl-piperazin-1-ylmethyl)-2-phenyl-quinoline-4-carboxylic acid((S)-1-cyclohexyl-ethyl)-amide |
| 37 | 3-[4-(2-Hydroxy-ethyl)-3-oxo-piperazin-1-ylmethyl]-2-phenyl-quinoline-4-carboxylic acid((S)-1-cyclohexyl-ethyl)-amide |
| | dihydrochloride |
| 38 | 8-(3-Oxo-piperazin-1-ylmethyl)-7-phenyl-2,3-dihydro-[1,4]dioxino[2,3-g]quinoline-9-carboxylic acid((S)-1-cyclohexyl-ethyl)- |
| | amide |
| 39 | 8-Fluoro-3-(3-oxo-piperazin-1-ylmethyl)-2-phenyl-quinoline-4-carboxylic acid((S)-1-cyclohexyl-ethyl)-amide |
| 40 | 3-(3-Oxo-piperazin-1-ylmethyl)-2-phenyl-quinoline-4-carboxylic acid((S)-1-phenyl-propyl)-amide |
| 41 | 3-(3-Oxo-piperazin-1-ylmethyl)-2-thiophen-2-yl-quinoline-4-carboxylic acid((S)-1-cyclohexyl-ethyl)-amide |
| 42 | 3-(3-Oxo-piperazin-1-ylmethyl)-2-phenyl-quinoline-4-carboxylic acid((S)-2-methyl-1-phenyl-propyl)-amide |
| 43 | 3-[3-Oxo-4-(2-piperidin-1-yl-ethyl)-piperazin-1-ylmethyl]-2-phenyl-quinoline-4-carboxylic acid((S)-cyclohexyl-ethyl)-amide |
| 44 | 2-(4-Fluoro-phenyl)-3-(3-oxo-piperazin-1-ylmethyl)-quinoline-4-carboxylic acid((S)-cyclohexyl-ethyl)-amide |
| 45 | 3-(3-Oxo-piperazin-1-ylmethyl)-2-(4-trifluoromethyl-phenyl)-quinoline-4-carboxylic acid((S)-cyclohexyl-ethyl)-amide |
| 46 | 2-(2-Fluoro-phenyl)-3-(3-oxo-piperazin-1-ylmethyl)-quinoline-4-carboxylic acid((S)-cyclohexyl-ethyl)-amide |
| 47 | 3-(3-Oxo-piperazin-1-ylmethyl)-2-phenyl-6-trifluoromethyl-quinoline-4-carboxylic acid((S)-1-cyclohexyl-ethyl)-amide |
| 48 | 3-(3-Oxo-piperazin-1-ylmethyl)-2-phenyl-7-trifluoromethyl-quinoline-4-carboxylic acid((S)-1-cyclohexyl-ethyl)-amide |

1. A compound of formula (I) below or a pharmaceutically acceptable salt or hydrate thereof. 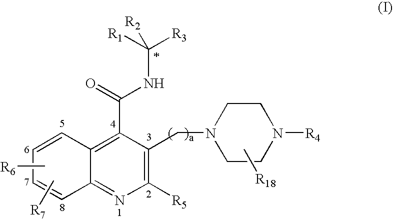
wherein:
R₁ is H or alkyl;
R₂ is aryl or cycloalkyl or heteroaryl, optionally substituted one or more times by alkyl, OH or alkoxy;
R₃ is H or alkyl or cycloalkyl or cycloalkylalkyl, optionally substituted one or more times by hydroxy or by one or more fluorines;
R₄ is H, or —R₈R₉ where R₈ is optionally substituted one or more times by R₁₃, or R₁₉;
R₈ is alkyl or alkenyl;
R₉ is S(O₂)R₁₀, S(O₂)OR₁₀, ONO, C(O)OR₁₀, C(O)NR₁₁R₁₂, or CN;
R₁₀ is H, alkyl, aryl or cycloalkyl;
R₁₁ and R₁₂ are independently selected from H and alkyl;
R₁₃ is R₁₄ or —R₁₄R₁₅;
R₁₄ is alkyl, aryl, cycloalkyl, arylalkyl, or a five-, six-, seven- or eight-membered heterocyclic ring comprising one or more heteroatoms selected from N, O and S;
R₁₅ is alkyl or —R₁₆COOR₁₇;
R₁₆ is a single bond or alkyl;
R₁₇ is H or alkyl;
R₁₈ is H or up to three oxo substituents;
R₁₉ is R₂₀ or —R₂₀R₂₁;
R₂₀ is alkyl, alkenyl or a single bond;
R₂₁ is OH, aryl, cycloalkyl or a saturated heterocyclic ring comprising one or more heteroatoms selected from N, O and S;
R₅ is a alkyl, cycloalkyl, cycloalkylalkyl, aryl, or single or fused ring aromatic heterocyclic group, which group may be substituted one or more times by halo, hydroxy, alkyl or alkyl substituted one or more times by halo or hydroxy;
R₆ represents H or up to three substituents independently selected from the list consisting of: alkyl, alkenyl, aryl, alkoxy or a hydroxylated derivative thereof, hydroxy, halogen, nitro, cyano, carboxy, alkylcarboxy, alkylcarboxyalkyl, haloalkyl such as trifluoromethyl, amino or mono- or di- alkylamino; or R₆ represents a bridging moiety which is arranged to bridge two adjacent ring atoms, which bridging moiety comprises alkyl or dioxyalkylene;
R₇ is H or halo;
a is 1-6; and
any of R₂, R₅, R₈, R₁₀, R₁₁, R₁₂, R₁₄, R₁₆, R₁₇ and R₂₁ may optionally be substituted one or more times by halo, hydroxy, amino, cyano, nitro, carboxy or oxo;
subject to the proviso that said compound is not a compound wherein R₇ represents H, R₅ represents unsubstituted phenyl, R₁₈ is H, and R₁, R₂, R₃ and
R₄ are one of the following combinations:

| 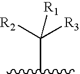 | a | R₄ | R₆ |
|---|---|---|---|
| 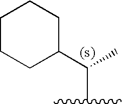 | 1 | 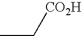 | H |
| 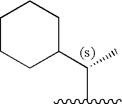 | 1 | H | H |
| 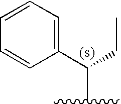 | 1 | H | H |
| 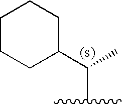 | 1 | 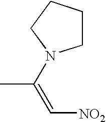 | H |
| 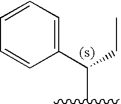 | 2 | 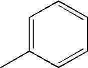 | H |
| 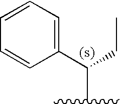 | 3 | 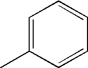 | H |
| 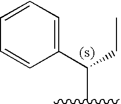 | 4 | 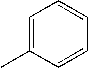 | H |
| 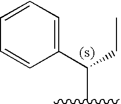 | 3 | 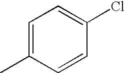 | H |
| 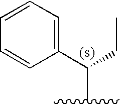 | 2 | 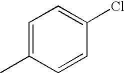 | H |
| 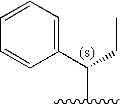 | 2 | 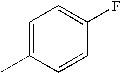 | H |
| 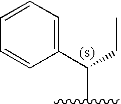 | 3 | 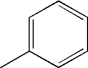 | OMe |
| 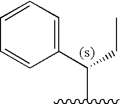 | 1 | 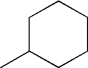 | H |
| 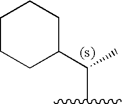 | 1 | 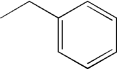 | H |
| 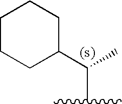 | 1 | 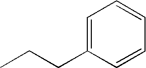 | H |
| 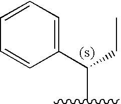 | 1 | 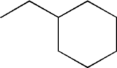 | H |
| 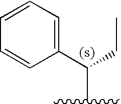 | 1 | 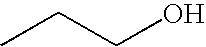 | H |
| 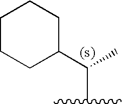 | 1 | 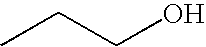 | H |
| 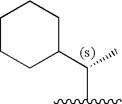 | 1 | Et | H |
| 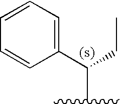 | 1 | Me | H |
| 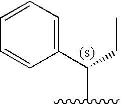 | 1 | 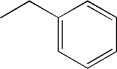 | H |
| 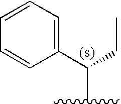 | 2 | Me | H |
| 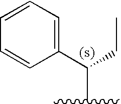 | 1 | Et | H |
| 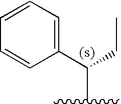 | 1 | 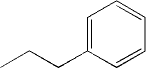 | H |
| 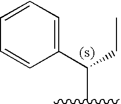 | 1 | 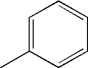 | H |

2. A compound as claimed in claim 1, wherein R₃ represents methyl, ethyl, isopropyl, cyclopropyl, hydroxymethyl or hydroxyethyl.

3. A compound as claimed in claim 1 or claim 2, wherein R₂ represents phenyl or cyclohexyl.

4. A compound as claimed in claim 3, wherein R₂ represents phenyl which is meta-or para-substituted once by —OMe or —OH.

5. A compound as claimed in any preceding claim, wherein R₁ is hydrogen or methyl.

6. A compound as claimed in any preceding claim, wherein R₅ is phenyl which is unsubstituted or which is substituted one or more times by halo such as fluoro and/or by haloalkyl such as trifluoromethyl.

7. A compound as claimed in any of claims 1-5, wherein R₅ is a heterocyclic ring, such as an unsaturated heterocyclic ring, comprising at least one heteroatom such as S.

8. A compound as claimed in claim 7, wherein R₅ is

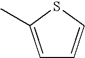

9. A compound as claimed in any preceding claim, wherein R₇ represents hydrogen.

10. A compound as claimed in any preceding claim, wherein R₆ represents hydrogen or one or more substituents selected from fluoro, chloro, bromo or trifluoromethyl.

11. A compound as claimed in claim 10, wherein each of said one or more substituents is respectively positioned at the 5′, 6′, 7′ or 8′ position around the quinoline ring of said compound.

12. A compound as claimed in any of claims 1-9, wherein R6 represents one ring substituent, which is hydroxy, alkoxy such as methoxy or ethoxy or a hydroxylated derivative thereof, alkoxycarboxylate such as methoxycarboxylate or ethoxycarboxylate or an esterified derivative thereof such as methoxyethanoate ethoxyethanoate, or alkoxyamido such as methoxyamido or ethoxyamido.

13. A compound as claimed in claim 12, wherein said one ring substituent is located at the 6 or 7 position around the quinoline ring of said compound.

14. A compound as claimed in any preceding claim, wherein a is 1, 2 or 3.

15. A compound as claimed in any preceding claim, wherein R₄ is hydrogen.

16. A compound as claimed in any preceding claim, wherein R₈ is methyl, ethyl, ethenyl or propenyl.

17. A compound as claimed in any preceding claim, wherein R₉ is C(O)OH or C(O)NH₂.

18. A compound as claimed in any of claims 1-16, wherein R₉ is S(O₂)R₁₀, S(O₂)OR₁₀, or C(O)OR₁₀, and R₁₀ is phenyl, methyl or ethyl.

19. A compound as claimed in any of claims 1-16, wherein R₉ is C(O)NR₁₁R₁₂ and each of R₁₀ and R₁₁ is the same one of methyl or ethyl.

20. A compound as claimed in any of claims 1-19, wherein R₄ is branched or linear R₈(R₁₃)R₉, R₁₃ is R₁₄ and R₁₄ is C₁₋₆ alkyl, or phenyl, or phenylmethyl, or phenylethyl.

21. A compound as claimed in claims 1-19, wherein R₄ is branched or linear R₈(R₁₃)R₉, R₁₃ is R₁₄R₁₅, and R₁₄ is a five- or six-membered saturated heterocyclic ring.

22. A compound as claimed in claim 21, wherein said heterocyclic ring comprises one or more N atoms.

23. A compound as claimed in claim 21, wherein said heterocyclic ring is N-linked to said R₈.

24. A compound as claimed in any of claims 1-19, wherein R₄ is branched or linear R₈(R₁₃)R₉, R₁₃ is —R₁₄R₁₅, and R₁₄ is C₁₋₆ alkyl, or phenyl, or phenylmethyl, or phenylethyl.

25. A compound as claimed in any of claims 21-24, wherein R₁₅ is hydrogen, methylethanoate, ethylethanoate, propylethanoate or butylethanoate.

26. A compound as claimed in any preceding claim, wherein R₂₀ is a single bond and R₂₁ is aryl such as phenyl.

27. A compound as claimed in any of claims 1-25, wherein R₂₀ is straight chain alkyl such as methyl, ethyl or propyl, and R₂₁ is OH, aryl, or a saturated heterocyclic ring comprising one or more N heteroatoms.

28. A compound as claimed in any preceding claim, wherein R₁₈ is H.

29. A compound as claimed in any of claims 1-27, wherein R₁₈ represents one or more oxo substituents.

30. A compound as claimed in claim 29, wherein R₁₈ represents one oxo substituent which is positioned at the 3′, 5′ or 6′ position around the piperazine ring of said compound.

31. A compound as claimed in claim 29, wherein R₁₈ represents two oxo substituents which are respectively positioned at the 3′ and 5′ or at the 3′ and 6′ positions around the piperazine ring of said compound.

32. A compound as claimed in any preceding claim, which is selected from the following:

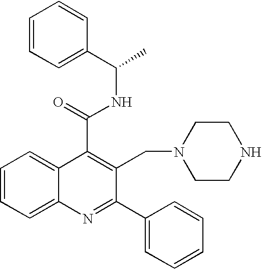

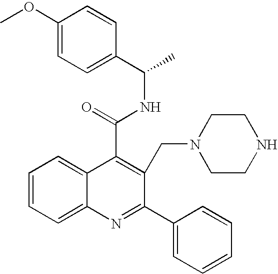

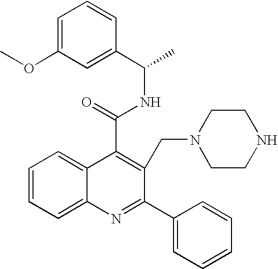

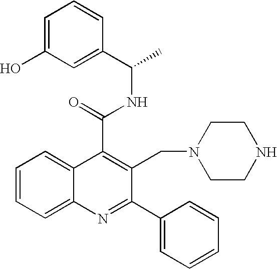

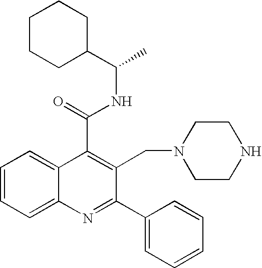

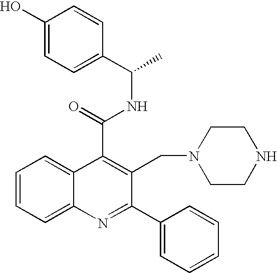

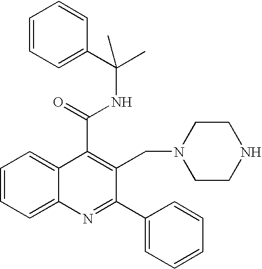

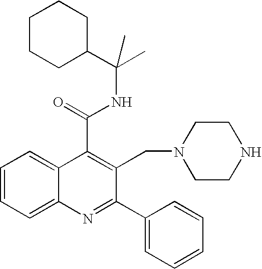

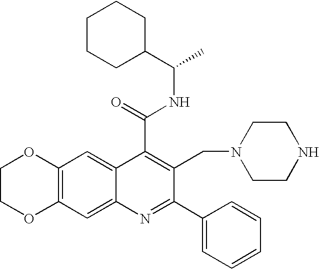

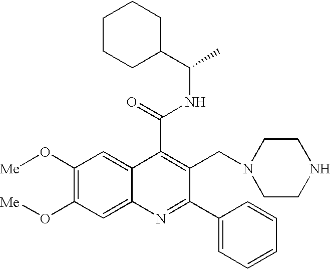

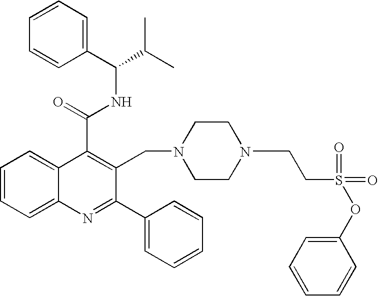

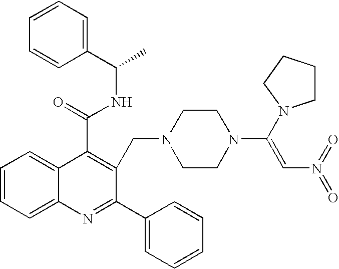

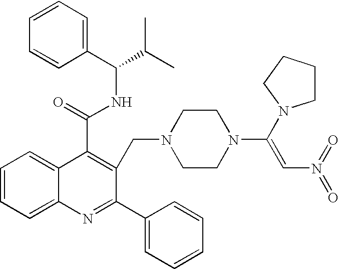

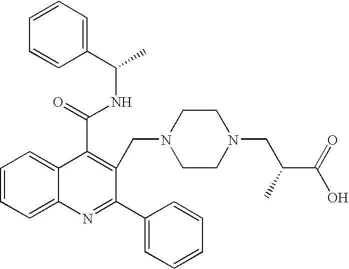

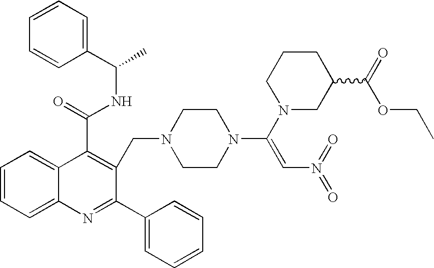

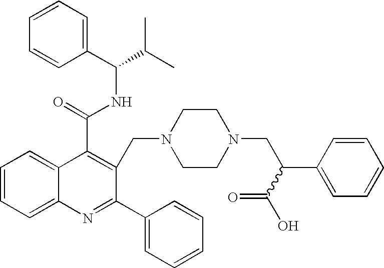

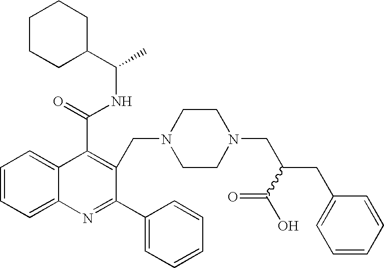

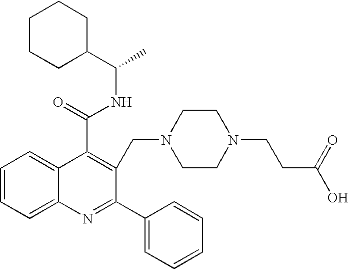

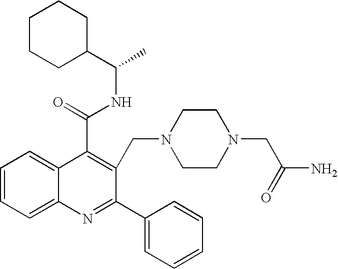

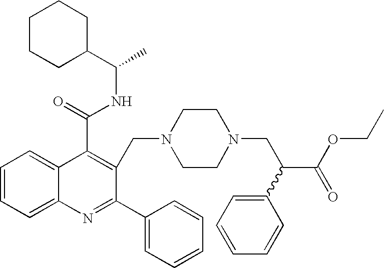

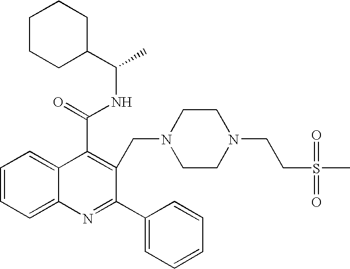

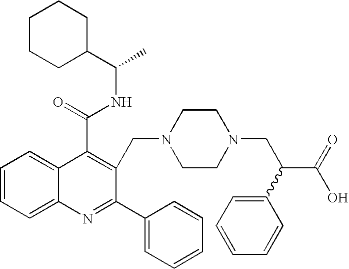

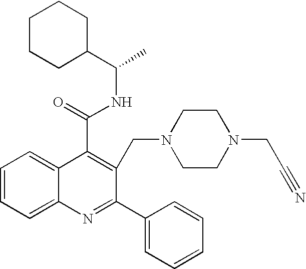

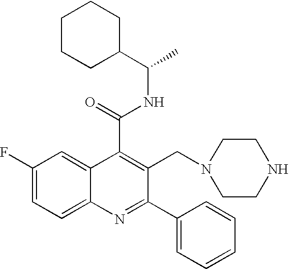

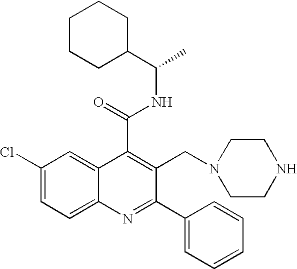

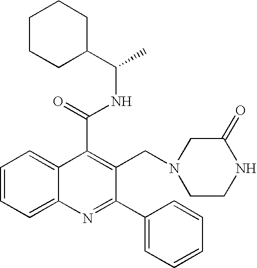

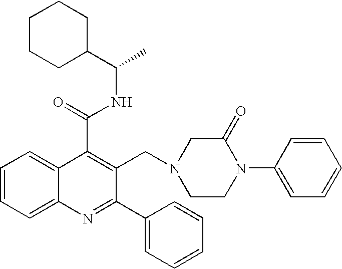

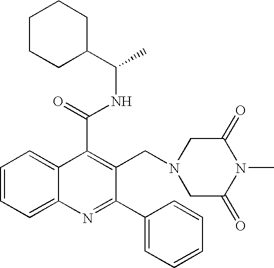

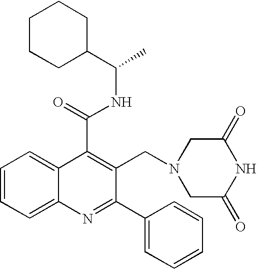

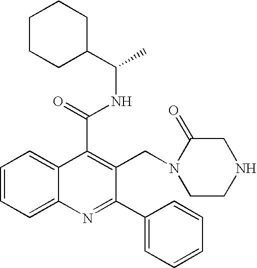

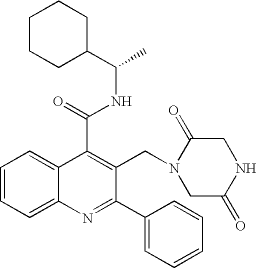

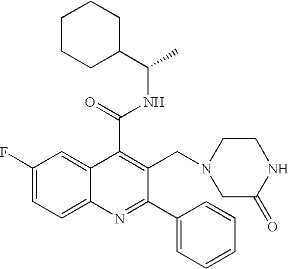

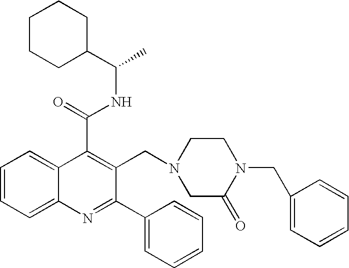

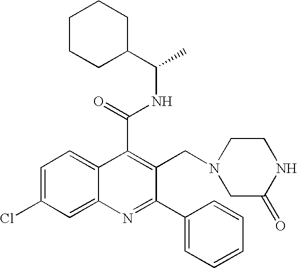

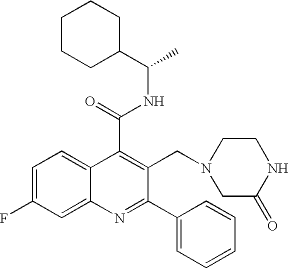

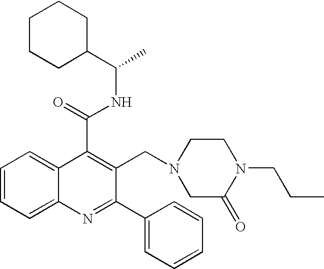

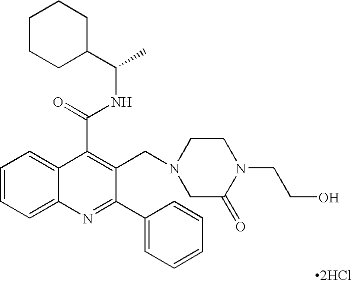

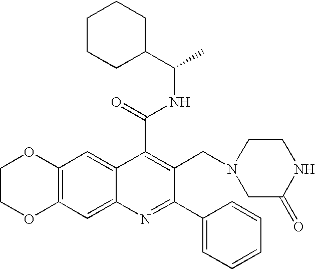

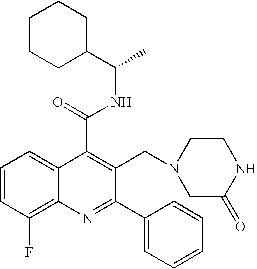

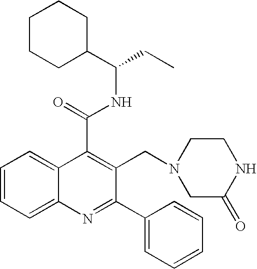

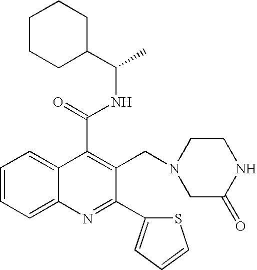

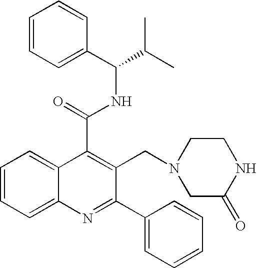

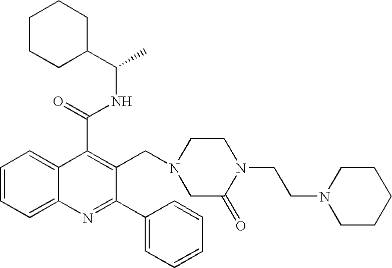

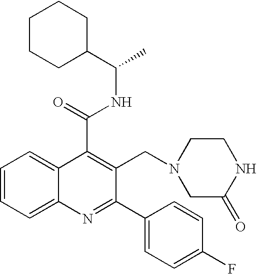

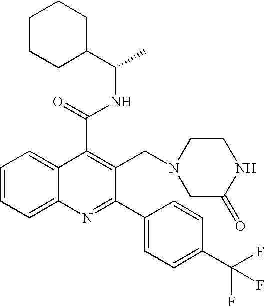

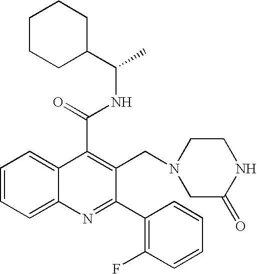

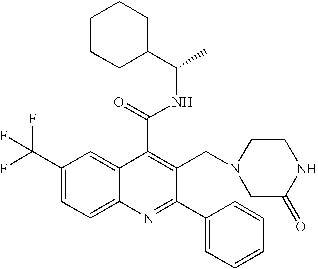

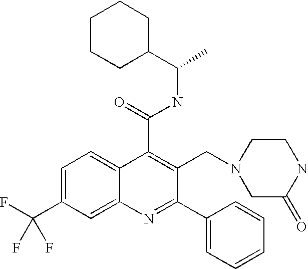

33. A process for the preparation of a compound of formula (I) according to any of claims 1-32, or a salt thereof and/or a solvate thereof, which process comprises reacting a compound of formula (II) or an active derivative thereof: 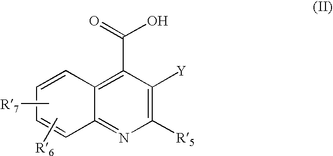
wherein R′₅, R′₆, and R′₇ are R₅, R₆, and R₇ respectively as defined in relation to formula (I) or a group convertible to R₅, R₆, and R₇ respectively, and Y′ is a group of formula (Y) or a group convertible thereto 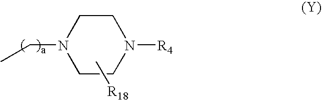
wherein R₄ and R₁₈ are defined as in relation to formula (I) above, with a compound of formula (III): 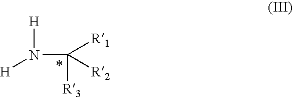
wherein R′₁, R′₂ and R′₃ are R₁, R₂ and R₃ as defined for formula (I) or a group or atom convertible to R₁, R₂ and R₃ respectively; to form a compound of formula (Ib): 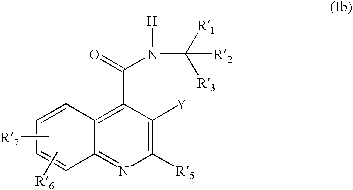
wherein R′₁, R′₂, R′3, R′₅, R′₆, R′₇ and Y′ are as defined above, and thereafter carrying out one or more of the following optional steps:
(i) converting any one of R′₁, R′₂, R′₃, R′₅, R′₆, R′₇ and Y′ to R₁, R₂, R₃, R₅, R₆, R₇ and Y respectively as required, to obtain a compound of formula (I);
(ii) converting a compound of formula (I) into another compound of formula (I); and
(iii) preparing a salt of the compound of formula (I) and/or a solvate thereof.

34. A process for the preparation of a compound of formula (I) according to any of claims 1-32, wherein a is 1, or a salt thereof and/or a solvate thereof, which process comprises reacting a compound of formula (T) or an active derivative thereof: 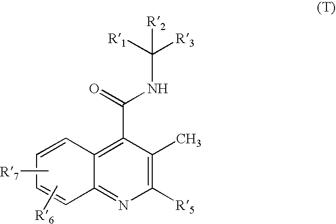
wherein each of R′₁, R′₂, R′₃, R′₅, R′₆, and R′₇ is R₁, R₂, R₃, R₅, R₆, or R₇ respectively as defined in relation to formula (I) or a group convertible to R₁, R₂, R₃, R₅, R₆, or R₇ respectively, providing that R₂ is not an aromatic group, with a compound of formula (W) 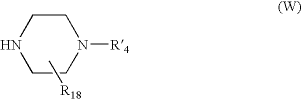
wherein R′₄ is a group R₄ as defined in relation to formula (I) or a protected form thereof or a group convertible thereto, and R₁₈ is a group R₁₈ as defined in relation to formula (I), to form a compound of formula (Ib): 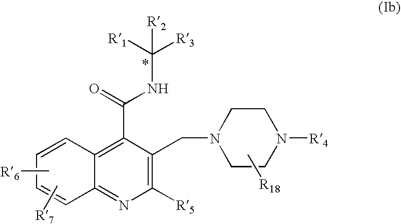
and thereafter carrying out one or more of the following optional steps:
(i) converting any one of R′₁, R′₂, R′₃,, R′₄, R′₅, R′₆, and R′₇ to R₁, R₂, R₃, R₄, R₅, R₆, and R₇ respectively as required, to obtain a compound of formula (I) as claimed in claim 1;
(ii) converting a compound of formula (I) as claimed in claim 1 into another compound of formula (I) as claimed in claim 1; and
(iii) preparing a salt of the compound of formula (I) as claimed in claim 1 and/or a solvate thereof.

35. A pharmaceutical composition comprising a compound of formula (I) according to any of claims 1-32, or a pharmaceutically acceptable salt or solvate thereof, and a pharmaceutically acceptable carrier.

36. A compound of formula (I), or a pharmaceutically acceptable salt or solvate thereof, for use as an active therapeutic substance.

37. A compound of formula (I), or a pharmaceutically acceptable salt or solvate thereof, for the treatment or prophylaxis of the Primary and Secondary Conditions.

38. Use of a compound of formula (I), or a pharmaceutically acceptable salt or solvate thereof, in the manufacture of a medicament for the treatment of the Primary and Secondary Conditions.

39. A method for the treatment and/or prophylaxis of the Primary and Secondary Conditions in mammals, particularly humans, which comprises administering to the mammal in need of such treatment and/or prophylaxis an effective, non-toxic pharmaceutically acceptable amount of a compound of formula (I) or a pharmaceutically acceptable salt or solvate thereof.
